(19) United States
(12) Patent Application Publication
(10) Pub. No.: US 2021/0247400 A1
(43) Pub. Date: Aug. 12, 2021

(54) ACTIVATION OF PATTERN-TRIGGERED IMMUNITY IN PLANTS BY LIPOOLIGOSACCHARID-SPECIFIC REDUCED ELICITATION (LORE) AND VARIANTS THEREOF

(71) Applicant: Technische Universität München, München (DE)

(72) Inventors: Stefanie RANF-ZIPPROTH, Haag a.d. Amper (DE); Ralph HÜCKELHOVEN, Freising (DE); Milena SCHAFFER, Freising (DE); Alexander KUTSCHERA, München (DE); Lars RAASCH, Freising (DE); Corinna DAWID, Freising (DE); Thomas HOFMANN, Neufahrn / Mintraching (DE)

(21) Appl. No.: 17/053,716

(22) PCT Filed: Feb. 15, 2019
(86) PCT No.: PCT/EP2019/053836

§ 371 Date: Nov. 6, 2020

(30) Foreign Application Priority Data
May 8, 2018 (EP) 18171342.1

(51) Int. Cl. G01N 33/68; A01N 43/56; A01N 43/74; A01N 43/80; A01N 47/18; A01N 47/20; A01N 47/34

(52) U.S. Cl. CPC G01N 33/6803; A01N 43/56; A01N 43/74; G01N 2333/415; A01N 47/18; A01N 47/20; A01N 47/34; A01N 43/80

(57) ABSTRACT

Presented herein, in part, are novel small compounds and uses thereof to analyze expression of LipoOligosaccharid-Specific Reduced Elicitation (LORE) polypeptides in plants and/or methods of inducing pattern-trigger immunity (PTI) in plants. Also presented herein are screening methods for identifying functional variants of LORE capable of activating pattern-triggered immunity (PTI) in a plant.

### RELATED PATENT APPLICATIONS

This patent application is a National Phase entry of, and claims priority to International Patent Application No. PCT/EP2019/053836 filed Feb. 15, 2019, entitled Activation Of Pattern-Triggered Immunity In Plants By LipoOligosaccharid-Specific Reduced Elicitation (Lore) And Variants Thereof, which claims priority to European Patent Application No. 18171342.1 filed May 8, 2018. The entire content of the foregoing patent applications is incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy, created on Oct. 28, 2020, is named “009848-0515857_AA2340_sequence_listing” and is 12 KB in size.

### FIELD

This technology relates, in part, to novel small compounds and uses thereof to analyze expression of LipoOligosaccharid-Specific Reduced Elicitation (LORE) polypeptides in plants and/or methods of inducing pattern-trigger immunity (PTI) in plants.

### SUMMARY

Presented herein, in certain aspects, is a method for determining whether a plant expresses the LipoOligosaccharid-specific Reduced Elicitation (LORE) represented by SEQ ID NO:1, or a functional variant thereof capable of activating pattern-triggered immunity (PTI), the method comprising the steps of: (a) contacting the plant, or a part thereof, with a compound of formula (I) and, subsequently, (b) determining whether PTI is activated, wherein the activation of PTI indicates that the plant expresses the functional LORE, or a functional variant thereof. In some aspects, presented herein is a screening method for identifying functional variants of LORE capable of activating pattern-triggered immunity (PTI) represented by SEQ ID NO:1, as well as a method of inducing pattern-triggered immunity (PTI) in a plant.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1: 3-hydroxy fatty acids (3-OH-FAs) trigger LORE-dependent immune responses in a chain length-specific manner. FIG. 1 shows Maximum [Ca²⁺]_{cyt} elevations upon treatment with LPS preparations (25 μg/mL; table 1) or water (mean SD, n=6) in aequorin-expressing wild-type (Col-0^{AEQ}) or lore-1 mutant seedlings (Panels A & B); Maximum [Ca²⁺]_{cyt} elevations in Col-0^{AEQ} seedlings treated with different concentrations of 3-OH—C10:0 (1) (Panel C) or 1 μM 3-OH-FAs of various chain length (mean SD, n=3) (Panel D); Maximum ROS accumulation in leaf discs of wild-type Col-0 or lore-5 mutant plants upon treatment with 3-OH-FAs of various chain length (1 μM; mean±SE, n=6) (Panel E); Maximum [Ca²⁺]_{cyt} elevations upon treatment with 1 μM 3-OH-FAs of various chain length in different lore mutants and complementation lines (CL, expressing pLORE:LORE in lore-1 background) (mean±SD, n=4) (Panel F); Defence gene expression in wild-type Col-0 or lore-5 mutant seedlings four hours after elicitation with 3-OH-FAs (1 μM) relative to Col-0 MeOH control. Individual data (symbols) and means (lines) of three experiments (Panel G); Activation of AtMPK3 and AtMPK6 in wild-type Col-0 or lore-5 mutant seedlings upon treatment with 3-OH-FAs (5 μM) or MeOH was analysed by immunoblot (IB) Panel (H) (Total protein staining shows equal loading); Pst DC3000 titre at 4 dpi in wild-type (Col-0^{AEQ}) or lore-1 mutant plants pre-treated with 10 μM 3-OH-FAs or MeOH for 3 days before infection (Panel J); and Maximum ROS accumulation in leaf discs from N. benthamiana transiently expressing LORE-GFP or kinase-inactive LOREm-GFP elicited with 1 μM 3-OH-FAs (mean±SE, n=8) (Panel K). Individual data (symbols) and means (lines) of two experiments are shown (n=6). Different letters indicate significant differences (one-way ANOVA with Tukey's post-test applied separately to Col-0^{AEQ} (small letters) and lore-1 (capitals), P<0.01). Experiments were repeated two (Panels F, K & J), three (Panels A-C, E & H) or four times (Panel D) with similar results.

FIG. 2: The 3-hydroxyl group is critical for LORE-mediated immune sensing of mc-3-OH-FAs. FIG. 2 shows Maximum [Ca²⁺]_{cyt} elevations in aequorin-expressing wild-type (Col-0^{AEQ}) or lore-1 mutant seedlings upon treatment with FAs (5 μM) or MeOH (mean±SD, n=6)(Panel A); Maximum ROS accumulation in leaf discs of wild-type Col-0 or lore-5 mutant plants upon treatment with FAs (5 μM; mean±SE, n=6) (Panel B); Maximum [Ca²⁺]_{cyt} elevations in wild-type Col-0^{AEQ} or lore-1 mutant seedlings upon treatment with FAs (5 μM) or MeOH (mean±SD, n=6)(Panel C); Maximum ROS accumulation in leaf discs of wild-type Col-0 or lore-5 mutant plants upon treatment with FAs (5 μM; mean±SE, n=8) (Panel D); Defence gene expression in wild-type Col-0 or lore-5 mutant seedlings four hours after elicitation with FAs (1 μM) relative to Col-0 MeOH control (Panel E) (Individual data (symbols) and means (lines) of three experiments are shown); Activation of AtMPK3 and AtMPK6 in wild-type Col-0 or lore-5 mutant seedlings treated with FAs (5 μM) or MeOH analysed by immunoblot (IB) (Panel F)(total protein staining shows equal loading); Maximum ROS accumulation in leaf discs from N. benthamiana transiently expressing LORE-GFP or kinase-inactive LOREm-GFP elicited with 5 μM FAs (mean±SE, n=8) (Panel G); and Maximum [Ca²⁺]_{cyt} elevations in wild-type Col-0^{AEQ} or lore-1 mutant seedlings upon treatment with the indicated compounds (5 μM) or MeOH (Panels H & J) (mean±SD, n=3 (H), n=6 (J)). Experiments were repeated two (Panels A-D) or three times (Panels G-J) with similar results.

FIG. 3: 3-OH-FAs in their free form show the strongest elicitor activity in Arabidopsis. FIG. 3 shows Maximum [Ca²⁺]c, elevations in wild-type Col-0^{AEQ} or lore-1 mutant seedlings upon treatment with the indicated compounds (5 μM) or MeOH (mean±SD, n=6) (Panels A, C-E); and Maximum ROS accumulation in leaf discs of wild-type Col-0 or lore-5 mutant plants upon treatment with the indicated compounds (5 μM; mean±SE, n=6) (Panel B). Experiments were repeated twice with similar results.

FIG. 4: LPS and HSL containing 3-hydroxyacyl building blocks do not activate LORE-mediated immune signalling. FIG. 4 shows [Ca²⁺]_{cyt} kinetics in aequorin-expressing wild-type (Col-0^{AEQ}) or lore-1 mutant seedlings upon treatment with (Panel A) Pci S400/S200 LPS (DOC-GPC; 50 μg/mL; table 1) or (Panel B) heat-detergent-repurified Pa H4 LPS (100 μg/mL; table 1), [after 30 min, flg22 or synthetic 3-OH—C10:0 (1) was added and measurements continued for 30 min (mean SD, n=3 each)]; ROS production in leaf discs of wild-type Col-0 or lore-5 mutant plants treated with heat-detergent-repurified Pa H4 LPS (100 μg/mL; table 1) (Panel C)[After 45 min, flg22 or synthetic 3-OH—C10:0 (1) was added and measurements continued for 45 min (mean SE, n=6 each)]; Maximum [Ca²⁺]_{cyt} elevations in wild-type Col-0^{AEQ} or lore-1 mutant seedlings upon treatment with the indicated compounds (5 μM; table 2) or solvent control (mean±SD, n=6) (Panel D); and Maximum ROS accumulation in leaf discs of wild-type Col-0 or lore-5 mutant plants upon treatment with the indicated compounds (5 μM; table 2; mean SE, n=6) (Panel E). Experiments were repeated two (Panels A,C-E) or three (Panel B) times with similar results.

FIG. 5: LPS containing 3-hydroxyacyl building blocks does not activate LORE-mediated immune signalling. FIG. 5 shows [Ca²⁺], kinetics in aequorin-expressing wild-type (Col-0^{AEQ}) or lore-1 mutant seedlings upon treatment with Pci S400/S200 LPS (DOC-GPC; 50 μg/mL; table 3). After 30 min, flg22 or synthetic 3-OH—C10:0 (1) was added and measurements continued for 30 min (mean SD, n=3 each). Experiment was repeated two times with similar results.

FIG. 6: LORE-dependent immune sensing of (R)-3-OH—C10:0 (44), (S)-3-OH—C10:0 (45), and Me-Gly-N-3-OH—C10:0 (46). FIG. 6 shows Maximum [Ca²⁺]_{cyt} elevations in Arabidopsis seedlings treated with different concentrations of (R)-3-OH—C10:0 (44) or (S)-3-OH—C10:0 (45) (mean SD, n=6) (Panel A)[Concentrations of the stock solutions were determined by quantitative NMR. Experiment was repeated four times with similar results]; and Maximum [Ca²⁺]_{cyt} elevations in Arabidopsis seedlings treated with the indicated compounds (5 μM; mean±SD, n=6) (Panel B)[Experiment was repeated two times with similar results].

FIG. 7. LPS preparations from a P. syringae pv. tomato DC3000 ΔpagL mutant contain free 3-OH—C10:0 and activate LORE-dependent PTI. FIG. 7 shows Maximum [Ca²⁺]_{cyt} elevations in Arabidopsis seedlings treated with the indicated LPS preparations of P. syringae pv. tomato DC3000 (Pst) wild type, a Pst ΔpagL mutant (25 μg/mL; Table 1), or water as control (mean SD, n=6). Experiment was repeated three times with similar results.

FIG. 8. Repurified LPS does not activate late PTI responses. FIG. 8 shows ROS production was measured over 40 h (left) in leaf discs treated with heat-detergent-repurified LPS from P. aeruginosa (Pa) H4 (25 μg/mL final concentration; Table 1) (mean±SE, n=6) (Panel A); and Induction of peroxidase activity in Arabidopsis leaf discs (n=12) 24 h after treatment with heat-detergent-repurified (RP) LPS from P. aeruginosa (Pa) H4 (25 μg/mL final concentration; Table 1) (Panel B). Different letters indicate significant differences (one-way ANOVA with Tukey's post-hoc test, p<0.05). Experiments were repeated two (Panel A) or four times (Panel B) with similar results.

### DETAILED DESCRIPTION

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Infestations with pathogenic microorganisms cause significant reductions in crop yields and, hence, considerable economic losses every year. Xylella fastidiosa, for instance, causes the Olive Quick Decline Syndrome currently devastating olive groves in Italy. The outcome of attempted pathogen infection of a host depends on its ability to sense the invading pathogen and rapidly mount an arsenal of countermeasures.

Bacterial infections of plants are difficult to control and their prevention through phytohygiene measures and choice of resistant plant varieties is crucial. Potential pathogens face an active surveillance and defence system in plants and modern plant breeding aims at exploiting this natural plant defence repertoire to produce resistant varieties. Conserved microbial signatures, so-called microbe-associated molecular patterns (MAMPs), betray the pathogen's presence to the host. Plants evolved diverse immune receptors to sense microbial invaders through MAMPs, microbial effectors, and cellular perturbations caused by microbes. MAMPs are generally common to a broad range of pathogens and are conserved due to their essential functions for the microorganism. MAMP-triggered defence responses are effective against a wide spectrum of pathogens and constitute a fundamental first layer of plant immunity called “Pattern-Triggered Immunity” (PTI; (Jones and Dangl, 2006). Adapted pathogens evolved effectors to subvert PTI through interfering with MAMP perception or execution of MAMP-triggered defence responses, and to facilitate successful colonization of a host plant, which is known as “Effector-Triggered Susceptibility” (ETS; (Lindeberg et al., 2012). Plants, in turn, developed a so-called “Effector-Triggered Immunity” (ETI), i.e. they evolved resistance (R) proteins to detect effectors or their activity. In an ongoing dynamic arms-race between host and pathogen, microorganisms can quite quickly evolve strategies to overcome R-gene mediated resistance (Jones and Dangl, 2006).

MAMPs are overall conserved and essential microbial structures that are detected by specific pattern-recognition receptors (PRRs). Cell surface components such as e.g. flagellin, peptidoglycan (PGN) and LPS are predestined as MAMPs because of their exposed position, their occurrence in whole microbial classes and their vital role for microbial survival. Both animals and plants have acquired the ability to recognize MAMPs and to trigger defence reactions and MAMP recognition systems appear to have emerged independently in plants and animals, as illustrated for example by the sensing of flagellin via distinct epitopes in plants and animals (Zipfel and Felix, 2005). Animals have evolved different extracellular and intracellular receptors for many MAMPs in parallel (Kieser and Kagan, 2017) and, similarly, plants independently evolved several receptors for certain MAMPs, e.g. flagellin, which is perceived through two distinct epitopes, flg22 and flgII-28, by different receptors (Ranf, 2017). In contrast to animals, all plant PRRs known to date reside at the cell surface and belong mainly to the class of receptor-like proteins (RLPs) or receptor-like kinases (RLKs). Both types of PRRs engage a variety of extracellular domains for binding of diverse ligands, such as leucine-rich repeat (LRR) or Lysin-motif (LysM) domains (Ranf, 2017). RLKs are transmembrane proteins that contain a cytoplasmic kinase domain for signal transduction, while RLPs, lacking enzymatically active domains, interact with other transmembrane or cytoplasmic proteins for intracellular signal relay. Generally, PRRs constitute the ligand-binding components of highly dynamic and tightly regulated multi-protein complexes (Macho and Zipfel, 2014).

Downstream of the specific PRRs, signal transduction pathways quickly converge as different MAMPs activate a common set of signalling and defence responses (Boller and Felix, 2009). Rapid induction of ion fluxes across the plasma membrane results in membrane depolarization and changes in the cytosolic concentration of the secondary messenger Ca²⁺([Ca²⁺]_{cyt}). Subsequently, the generation of reactive oxygen species (ROS) through the plasma membrane-resident NADPH oxidase, RBOHD, directly confines pathogen spread via toxic effects and cell wall strengthening. ROS in turn induce [Ca²⁺]_{cyt} elevations, resulting in local signal amplification and/or systemic signalling via a self-amplifying calcium-ROS-circuit (Miller et al., 2009; Ranf et al., 2011; Dubiella et al., 2013). Mitogen-activated protein kinase (MAPK) cascades and Ca²⁺-dependent protein kinases (CDPKs) (in)activate multiple substrates to modulate metabolic processes or gene expression (Tena et al., 2011). MAMPs induce such local responses, but also the establishment of resistance in distant tissues via salicylic acid (SA) or jasmonic acid/ethylene signalling (Pieterse et al., 2012). Many genes encoding for PRRs and MAMP-activated signalling components are systemically up-regulated, thus enhancing MAMP sensitivity and PTI signalling also in yet uninfected tissues (Boller and Felix, 2009). Ultimately, MAMP detection leads to the local and systemic production of a myriad of antimicrobial secondary metabolites and pathogenesis-related (PR) proteins, which are the actual executers of the plant defence system. PR proteins include diverse classes of antimicrobial peptides (AMPs, e.g. α/β-thionins, defensins, cyclotides) and lipid-transfer proteins (LTPs), as well as enzymes with antimicrobial activities, such as chitinases, lysozymes or lipases (Van Loon and Van Strien, 1999; Barbosa Pelegrini et al., 2011; Spoeland Dong, 2012).

The bacterial cell envelope is a complex structure that provides stability and shields the cell from its surroundings. In Gram-negative bacteria the cell envelope is built up by a two membrane system with a specialized asymmetric outer membrane (OM), the inner leaflet of which consists mainly of phospholipids, while up to 75% of the outer leaflet is made up of the glycolipid lipopolysaccharide (LPS), i.e. ˜10⁶ molecules of LPS are present per bacterial cell. LPS consists of three covalently linked domains with different chemical and biological properties: the lipophilic lipid A (LA) moiety, the hydrophilic oligosaccharide (OS) core region and the O-polysaccharide (OPS; Alexander and Rietschel, 2001).

The typical enterobacterial LA consists of a di-phosphorylated di-glucosamine with four primary and two secondary fatty acids (all C12/14) attached in an asymmetric fashion. The fatty acids are embedded in the OM and the di-glucosamine is linked to the core OS composed of about 10 to 15 monosaccharides. The primary stability and barrier functions of LPS are conferred by the rather conserved inner core-LA region (Whitfield and Trent, 2014). Cross-linking of the negative residues of the inner core and LA backbone through divalent cations (Mg²⁺, Ca²⁺) is crucial for the tight packing of the charged LPS molecules, which in turn is fundamental to both rigidity and low permeability of the OM (Alexander and Rietschel, 2001).

The core OS is conceptually subdivided into the variable outer core and the more conserved inner core region that usually contains heptose (Hep) and the LPS-specific monosaccharide 3-deoxy-D-manno-oct-2-ulosonic acid (Kdo; Alexander and Rietschel, 2001). In most cases, the OPS attached to the core region is the O-specific antigen (OSA). The OSA is built up by a varying number of repetitive units composed of up to five monosaccharides. Its composition is highly diverse among bacterial species and strains, determining their serological and antigenic specificity. Other polysaccharides such as capsular polysaccharides (CPS), the common polysaccharide antigen (CPA) of P. aeruginosa or the enterobacterial common antigen (ECA), specific to Enterobacteriaceae, can also be found instead of, or in parallel with, the OSA (Raetz and Whitfield, 2002).

The OPS chains shield the bacterium from unfavourable environments by providing a steric as well as diffusion barrier, e.g. to antibacterial agents that target the interior core and LA parts of LPS (Alexander and Rietschel, 2001). In addition to the form of LPS carrying an OPS (also referred to as “smooth” LPS), a further form lacking the OPS (referred to as “rough” LPS) is also known and both forms often occur in parallel (Alexander and Rietschel, 2001). Bacteria lacking long-chain OPS or negative core charges on their LPS are known to be generally non-virulent and cannot persist under stress conditions, such as within a host (Raetz and Whitfield, 2002). Due to their outermost localization, OPS mediate adhesion to host surfaces and possibly host selectivity (Bogino et al., 2013). Through adherence to other bacteria they are also involved in formation of biofilms, which likely is the preferred growth mode within host tissue (Dongari-Bagtzoglou, 2008; Bogino et al., 2013).

The length of the OPS chain can range from one to >100 repeats and the number and length of fatty acids on the LA as well as phosphorylation, acylation and various other non-stoichiometric modifications on the LA, core OS or OPS can also differ considerably (Alexander and Rietschel, 2001). Moreover, LPS is not a static structure but highly dynamic and can be modified in manifold ways (Raetz et al., 2007; Needham and Trent, 2013).

In summary, LPS structures vary substantially between different bacterial species, likely due to adaptations to different environments and lifestyles, but also a single bacterial cell envelope intrinsically comprises a complex mixture of different LPS variants with remarkable size heterogeneity.

LPS from different bacterial species has been shown to act as MAMP in various plant species (Newman et al., 2013). LPS from several bacteria and enterobacterial LA, for instance, induce NO production in Arabidopsis (Zeidler et al., 2004). Oligo-rhamnan containing OPS, found in many phytopathogenic bacteria, Xanthomonas core OS and LA as well as Burkholderia LPS and LA trigger defence-related gene expression in Arabidopsis and/or ROS production in tobacco (Bedini et al., 2005; Silipo et al., 2005; Madala et al., 2012). LPS also plays a role in induced systemic resistance by plant growth-promoting rhizobacteria and in nodule formation and colonization in the Rhizobium-legume symbiosis (Newman et al., 2013). Thus, LPS is not only involved in pathogenic but also in beneficial plant-microbe interactions. Apparently, plants are capable of sensing different parts of LPS or structural different epitopes within the same LPS moiety, however, the underlying perception mechanisms for the different LPS motifs are not yet understood and, accordingly, the understanding of the diverse roles of LPS in plant-bacteria interactions is still fragmentary.

In mammals, all three LPS domains contribute to immune recognition. The highly immunogenic OSA triggers antibody production in the adaptive immune system causing a selective pressure that presumably led to its extensive diversification (Whitfield and Trent, 2014). P. aeruginosa LPS is specifically recognized and internalized through its outer core OS by cystic fibrosis transmembrane conductance regulator (Schroeder et al., 2002). LPS is also recognized in picomolar concentrations as MAMP by the innate immune system through the LA moiety and induces inflammation. An exaggerated immune reaction to LPS/LA, also termed endotoxin, can result in a life-threatening multi-organ failure, the septic shock (Alexander and Rietschel, 2001; Tan and Kagan, 2014). LPS/LA binding to a preformed hetero-dimer of the LRR-type RLP Toll-like receptor 4 (TLR4) and soluble myeloid differentiation factor 2 (MD-2) leads to association with another TLR4/MD-2-dimer into a multimeric complex (Park et al., 2009). Subsequent dimerization of the intracellular TIR domain of the membrane-spanning TLR4 initiates intracellular signalling by recruiting adaptor proteins and activating protein kinases. LPS binding to TLR4/MD-2, however, is not a diffusion event but rather a process employing an intermolecular LPS transfer cascade involving LPS-binding protein (LBP) and the glycoprotein CD14, which occurs as soluble and membrane-anchored version. The high-affinity LBP can directly extract the membrane-bound LA moiety from the bacterial membrane, thus making LPS/LA available for host perception. LBP and CD14 are further required for LPS clearing and signal attenuation (Tan and Kagan, 2014). Intracellular LPS/LA is further sensed through LPS-mediated oligomerization and activation of inflammatory caspases (Shi et al., 2014). Gram-negative bacteria naturally release OM vesicles consisting of LPS and other PAMPs like flagellin, which synergistically activate immune responses (Ellis et al., 2010; Schwechheimer and Kuehn, 2015). Ultimately, LPS induces pro-inflammatory cytokines and interleukins, ROS and nitric oxide (NO) production and secretion of cationic antimicrobial peptides (CAMPs; (Tan and Kagan, 2014).

In mammals, the efficiency of immune sensing of LPS has been shown to depend on the three-dimensional conformation of the LA moiety which is determined by its primary structure (Seydel et al., 2000; Park et al., 2009). Whereas enterobacterial LA is a strong agonist of the TLR4/MD-2 and the caspase pathway, other LA variants are less potent stimulators or even antagonists. Indeed, many pathogens employ alterations of their LA structure as a virulence strategy to avoid immune recognition by the host (Needham and Trent, 2013). According to the different lifestyles, LPS modifications and the causal genes vary greatly between bacterial species. Some bacteria constitutively synthesize LPS/LA differing from the canonical enterobacterial LA, for instance with less and/or shorter acyl chains, e.g. Pseudomonas spp. Additionally, bacteria can dynamically remodel their LPS structure during and post-synthesis by various means. Such remodulations are controlled by transcriptional and post-translational mechanisms that enable bacteria to quickly adapt to changing and often hostile environments, e.g. within a host. The majority of the modifications known to date modulate the LA domain and the inner core (Needham and Trent, 2013). These modulations influence the physicochemical properties of the OM but are also important for pathogenesis. For example, cross-linking of negative charges on the inner core-LA region by divalent cations is vital for OM function. Furthermore, negative charges are crucial for LPS-receptor interactions and are targeted by host-derived CAMPs to disturb OM integrity. CAMPs represent an ancient defence strategy occurring in most eukaryotic hosts (e.g. insects, vertebrates, plants) and likely have driven the evolution of LPS structure modulations (Miller et al., 2005). Indeed, bacteria can mask negative LA/core OS charges by attaching cationic moieties such as 4-amino-4-deoxy-L-arabinose (Ara4N) or phosphoethanolamine (EtnP; Needham and Trent, 2013). The majority of LA modifications are controlled by the two-component regulatory systems (TCS) PhoP/PhoQ and PmrA/PmrB that are widespread in animal as well as plant pathogenic bacteria (Needham and Trent, 2013). TCS consist of a sensor kinase and a response regulator for signal relay that e.g. functions as transcription factor and translates external stimuli directly into gene expression changes (Lavin et al., 2007). PhoP/PhoQ and PmrA/PmrB both sense environmental stimuli such as acidic pH, low Mg²⁺ concentrations or CAMPs, and partially redundantly regulate LPS modifier genes along with other virulence factors (Chen and Groisman, 2013; Needham and Trent, 2013). Taken together, conditional LPS modifications directly influence pathogenesis in diverse hosts by altering the surface charge and permeability of the OM, enhancing resistance to antibacterial compounds and/or interfering with LPS immune sensing.

Recently, it has been shown by using Arabidopsis plants carrying the calcium reporter aequorin (Col-0^{AEQ}) that MAMPs rapidly induce characteristic [Ca²⁺]_{cyt} elevations (Ranf et al., 2008; Ranf et al., 2011). Arabidopsis Col-0^{AEQ} seedlings were also used to assess whether LPS preparations induce characteristic [Ca²⁺]_{cyt} elevations similar to other well-studied MAMPs. Interestingly, LPS preparations from diverse Pseudomonas spp. activate characteristic PTI responses, such as e.g. ROS accumulation, MAPK activation and expression of PTI-related genes, via the plant-specific bulb-type lectin receptor kinase LipoOligosaccharide-specific Reduced Elicitation (LORE)/S-Domain-1-29 (SD1-29) (Ranf et al., 2015). LPS from other phytopathogens such as Xanthomonas campestris and enterobacteria such as Escherichia coli, Salmonella enterica and Burkholderia spp., which are strong agonists of TLR4/MD-2-mediated immunity in mammals (Alexander and Rietschel, 2001) were also analysed. Strikingly, only Xanthomonas LPS, but not enterobacterial LPS, was found to activate typical PTI signalling in a LORE-dependent manner. Apparently, a specific LPS pattern common to Pseudomonas and Xanthomonas LPS but distinct in enterobacterial LPS is critical for its recognition by LORE. Further analysis revealed that the (inner core-)LA moiety of Pseudomonas and Xanthomonas LPS is sensitively detected as MAMP in Arabidopsis via LORE, and that the detected epitope is distinct from the enterobacterial LA moiety sensed in mammals (Ranf et al., 2015).

Despite the fact that a lot of effort is currently being invested into understanding the mechanisms behind plant responses to pathogens, there is still a need to identify suitable tools for inducing pattern-triggered immunity in plants. One focus currently lies on LPS, however, the majority of the LPS-induced plant responses reported so far require high, non-physiological LPS levels of between 50 to 100 μg/ml. These observations raise doubts with regard to the specificity of the responses analysed (Zipfel and Felix, 2005) and render it difficult to translate these results into agriculturally useful tools for protecting plants from pathogens. Accordingly, there is still a need to provide means and methods to enhance the protective activity of plants against pathogen attacks.

This need is addressed by the provision of the embodiments characterized in the claims.

Accordingly, the present invention relates to a method for determining whether a plant expresses the LipoOligosaccharid-specific Reduced Elicitation (LORE) represented by SEQ ID NO:1, or a functional variant thereof capable of activating pattern-triggered immunity (PTI), the method comprising the steps of: (a) contacting the plant, or a part thereof, with a compound of formula (I):

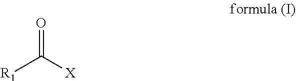

or a salt thereof,
wherein
R₁ is selected from —OH, —H, —OCH₃, —OCH₂CH₃,
—O—(CH₂)₂₋₁₁—CH₃, wherein one hydrogen atom in the —(CH₂)₂₋₁₁— group may be replaced by a —CH₃ group,

—O—(CH₂)₁₋₁₂—OH, wherein one or two hydrogen atoms in the —(CH₂)₁₋₁₂— group may be replaced by a —CH₃ group,

—O—(CH₂)₁₋₁₁—COOH, wherein one hydrogen atom in the —(CH₂)₁₋₁₁— group may be replaced by a —CH₃ group,

—NH₂, —SH, —SCH₃, —SCH₂CH₃,

an amino acid residue, preferably selected from Gly, Ala, Val, Leu, lie, Met, Thr, Ser, Cys, Gln, Asn, Glu, Asp, Arg or Lys, most preferably selected from Gly, Ala, Ser, Thr, Asp, Glu or Leu, which is attached via an amino group to form an amide bond with the carbonyl group of formula (I) and wherein the carboxyl group of the amino acid residue may be converted into an ester group, preferably a C₁-C₆ alkyl ester, more preferably a methyl ester, and a biogenic amine, preferably selected from putrescine, cadaverin, agmatine, spermidine or spermine, which is attached via an amino group to form an amide bond with the carbonyl group of formula (I);

and X is selected from one of formulas (II) and (III):

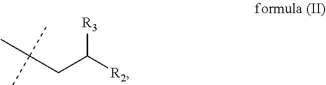

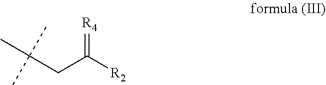

wherein
R₂ is —(CH₂)₄₋₈—CH₃;
R₃ is selected from —OH, —SH, —OCOCH₂CHOH(CH₂)₄₋₈CH₃; and
R₄ is selected from ═O, ═S, ═NH, and ═CH₂;
and the dashed line in formula (II) and (III) marks the bond which attaches X to the remainder of the compound of formula (I)
or a precursor thereof, and, subsequently, (b) determining whether PTI is activated, wherein the activation of PTI indicates that the plant expresses the functional LORE, or a functional variant thereof.

Any plant of interest can be subjected to the method of the present invention. Of particular interest in accordance with the present invention are agricultural crops, as these plants are of high economic interest. In accordance with the present invention, the plant can be selected from the group consisting of monocotyledonous plants and dicotyledonous plants. The term “monocotyledonous plants” refers to a group of plants that is characterized by having one seed-leaf (cotyledon), while the term “dicotyledonous plants” refers a second group of plants characterized by having two embryonic leaves. Non-limiting examples of monocotyledonous plants include wheat, oats, millet, barley, rye, maize, rice, sorghum, triticale, spelt and sugar cane while non-limiting examples of dicotyledonous plants include Arabidopsis, fibre plants (cotton, flax, hemp, jute), buckwheat, vines, tea, hops, pistachio, cress, linseed, oil plants (rape, mustard, poppy, olives, sunflowers, coconut, castor oil plants, cocoa beans, groundnuts), vegetables (e.g. spinach, lettuce, asparagus, cabbages, carrots, onions, tomatoes, potatoes, paprika, brassicas), aubergines, corn, tobacco, tagetes, calendula, cucumber plants (such as cucumber, marrows, melons), soft fruit (e.g. apples, pears, plums, peaches, almonds, cherries, strawberries, raspberries and blackberries), citrus fruit (such as oranges, lemons, grapefruit, mandarins), pumpkin/squash, courgette, beet (e.g. sugar beet and fodder beet), drupes (e.g. coffee, jujube, mango, palms such as e.g. date palms), lauraceae (e.g. avocados, cinnamon, camphor), ornamentals (e.g. flowers, shrubs, deciduous trees and conifers) and legumes (such as beans, lentils, peas, soybeans). Preferably, the plant is selected from Arabidopsis or other plants of the family Brassicaceae.

In accordance with the present invention, the plants can be traditional crop plants or plant varieties having new properties, which have been obtained by breeding with conventional methods, mutagenesis or by recombinant DNA techniques. Thus, the plants may include transgenic plants and plant hybrids.

In accordance with this method of the present invention, it is determined whether a plant of interest expresses the protein LipoOligosaccharid-specific Reduced Elicitation, referred to herein as “LORE”, or a functional variant of LORE. More specifically, the method of the present invention serves to determine whether the plant of interest expresses LORE, or a functional variant thereof, in its functional form that is capable of activating pattern-triggered immunity (PTI).

LORE is a bulb-type (B type) lectin receptor-like kinase (Ranf et al., 2015) belonging to the “S domain-1 (SD1)” multi-gene family with 32 members in Arabidopsis (Shiu and Bleecker, 2003). LORE has the database accession number At1 g61380 and the protein sequence of LORE is represented by SEQ ID NO:1, while the coding sequence of LORE is represented by SEQ ID NO:2.

As mentioned, the term “a functional variant thereof”, as used herein, relates to a functional variant of LORE as represented by SEQ ID NO:1. Non-limiting examples of such variants include different wild type forms and alleles of LORE, e.g., allelic variants, as well as homologues and analogues of LORE and mutant forms of LORE, as long as the variant is a functional variant. The term “functional variant” means that the variant retains or essentially retains the function of LORE as represented by SEQ ID NO:1 of being capable of activating pattern-triggered immunity (PTI). In particular, said function is the capability of activating pattern-triggered immunity in a plant in response to a compound of formula I, preferably the capability of activating pattern-triggered immunity in a plant in response to 3-hydroxydecanoid acid and more preferably the capability of activating pattern-triggered immunity in a plant of the family Brassicaceae, preferably Arabidopsis, in response to 3-hydroxydecanoid acid. Means and methods for analyzing this function are well known in the art and are discussed in detail herein below. “Essentially retains” means that said function is retained to at least 50%, preferably to at least 70%, more preferably to at least 80%, such as to at least 90%, and most preferably to at least 95%.

Preferably, the PTI is an immunity against pathogens selected from the group consisting of bacteria, fungi, oomycetes, viruses/viroids, nematodes, and pests, such as aphids or caterpillars. Particularly preferred bacterial pathogens are selected from the group consisting of Pseudomonas, such as e.g. P. syringae, P. aeruginosa, P. viridiflava, P. cichorii, P. savastanoi, P. avellanae or P. corrugata; xanthomonads, such as for example X. campestris, X. oryzae, X. axonopodis or X. vesicatoria; Xylella (for example X. fastidiosa); Candidatus Liberibacter spp.; Erwinia spp. (for example E. amylovora); Pectobacterium spp. (for example P. carotovorum and P. atrosepticum); Clavibacter michiganensis; Ralstonia spp. (e.g. R. solanacearum), or Dickeya spp. (e.g. D. dadantii and D. solani); Phytoplasma spp. or Spiroplasma spp. Particularly preferred basidiomycete fungi include e.g. Ustilago spp., Hemileia spp., Rhizoctonia spp., Puccinia spp. or Phakopsora spp. (e.g. Phakospora pachyrhizi). Particularly preferred ascomycete fungi include e.g. Fusarium spp., Blumeria spp., Verticillium spp., Alternaria spp., Erysiphe spp., Monilinia spp., Uncinula spp., Sclerotinia spp., Ramularia spp., Thielaviopsis spp., Botrytis spp. (e.g. Botrytis cinerea), Zymoseptoria tritici, Magnaporthe spp. (e.g. M. grisea and M. oryzae), Venturia spp., Podosphaera spp., Colletotrichum spp., Curvularia spp., Bipolaris spp., Pyrenophora spp., Piricularia spp. or Cercospora spp. Particularly preferred oomycetes include e.g. Hyaloperonospora spp. (e.g. H. arabidopsidis, H. brassicae or H. parasitica), Phytophthora spp. (e.g. Phytophthora nicotianae and Phytophthora infestans), Phythium spp., Albugo spp., Plasmopara viticola, or Peronospora tabacinae. Particularly preferred viruses include e.g. the mosaic viruses (e.g. tobacco, cucumber, cauliflower, or african cassava mosaic virus), leafroll viruses (e.g. potato leafroll virus, tomato yellow leaf curl virus), tomato spotted wilt virus, potato virus Y and X, plum pox virus, brome mosaic virus, Citrus tristeza virus, barley yellow dwarf virus, and tomato bushy stunt virus.

In a first step of the method of the present invention, a plant, or a part thereof, is contacted with a compound of formula (I).

As used herein, the terms “a plant” or “the plant” refers to the entire plant. Also encompassed herein is that a plurality of plants is subjected to the methods of the present invention, preferably simultaneously. The term “a part thereof”, as used herein with regard to a plant, refers to any part of a plant, preferably a part selected from seedlings, leaf discs, leaves, stems, branches, roots, cells, protoplasts, flowers and fruits. More preferably, in this method of the invention of determining whether a plant expresses LORE, seedlings or leaf discs are employed.

In accordance with the present invention, the plant(s) can be contacted by any method known in the art. In a preferred embodiment of the method of the invention, the plant(s) is/are contacted by spraying, dusting, scattering, coating or pouring.

Accordingly, the compound of formula (I) can be provided in a form selected from directly sprayable or dilutable solutions, including e.g. aqueous solutions, emulsifiable concentrates, coatable pastes, dilute emulsions, wettable powders, soluble powders, dusts, granulates, encapsulations in e.g. polymeric substances and natural or synthetic substances impregnated with the active compound. For use as a spray, the compound of formula (I) can, for example, be provided in liquid form dispersed in a gas, such that small droplets are formed. The spray then enables the distribution of the compound over a surface area, such as for example a single plant or a field comprising a plurality of plants. The dispersion of the compound in a gas is also referred to as atomizing. The compound in a liquid state may also be scattered onto plants or a field or may be poured onto the plants or a field. Furthermore, parts of the plant or entire plants can be coated with the compound of the present invention, for example by dipping the plant into the compound or by brushing the plants, or parts thereof, with the compound. Alternatively, or additionally, the compound can be applied by dusting, i.e. the (aerial) application of the compound in powder form. Furthermore, the compound can also be introduced into the soil on which the plants are growing, for example in form of a liquid, granules, pellets or a stick, which can e.g. disintegrate with time in order to release the compound used in accordance with the invention. The above described means can be chosen to achieve the desired route of application, such as e.g. to achieve a foliar application, application to the stem or buds, application to (and uptake through) the roots in case of application to the soil or application to the seeds or seedlings. It will be appreciated that the particular method of application has to be selected depending on the respective circumstances and the target of the treatment.

In accordance with the present invention, the term “the compound of formula (I)” does not encompass larger molecules, such as e.g. LPS or lipid A, even if these molecules comprise a structure of formula (I) as part of their overall structure. In other words, the compound of formula (I) consists of the structure shown in formula (I). Moreover, when the compound of formula (I) is obtained from natural sources, contamination with other molecules such as e.g. LPS or lipid A may sometimes be observed. In accordance with the present invention, it is preferred that the compound of formula (I) is provided in a purified form, i.e. a form that is free of contaminating molecules, most preferably the compound of formula (I) is free of LPS and lipid A.

In an alternative embodiment, the contacting of the plant in step (a) is carried out with a precursor of the compound of formula (I). It will be appreciated that said precursor needs to be chosen such that it can release the compound of formula (I) upon exposure to the respective plants. Non-limiting examples of such precursor molecules include 3-hydroxy-decanethioic acid (Norris and Bloch, 1963), 3-hydroxy-decanoic acid-CoA, 3-hydroxydodecanoic acid-CoA (Park et al., 2015), hydroxyacyl-ACP (Abdel-Mawgoud et al., 2010b), or homopolymer of 3-hydroxy-decanoic acid (Kalscheuer et al., 1999). All definitions and preferred embodiments provided herein with regard to employing the compound of formula (I) in the claimed methods apply mutatis mutandis when the precursor of formula (I) is employed.

Preferably, the contacting in step (a) is carried out with the compound of formula (I).

Methods for obtaining the compound of formula (I), or the precursor thereof, are well known in the art and include, without being limiting, the isolation of naturally occurring 3-hydroxy fatty acids (3-OH-FAs), for example from bacteria or synthesis of the compound of formula (I). Methods for the synthesis of such compounds are known to the skilled person and have been described in the appended examples as well as, e.g., in (Tahara and Mizutani, 1978), (Nakahata et al., 1982), (Deng et al., 1985), (Helmchen et al., 1985), (Burke et al., 1989), (Duthaler et al., 1989), (Aoyagi et al., 1996), (Fukuzawa et al., 2000), (Guo-Qiang et al., 2001), (Matsumoto et al., 2001), (Ashby et al., 2002), (Roo et al., 2002), (Poirier et al., 2002), (Solaiman et al., 2002), (Sánchez et al., 2003), (Wu et al., 2003); (Jaipuri et al., 2004), (Sanguanchaipaiwong et al., 2004), (Zheng et al., 2004), (Ward and O'Connor, 2005), (Concellón and Concellón, 2006), (Teixidó et al., 2007), (Wang et al., 2011), (Wu et al., 2011), (Adamus et al., 2012), (Chung et al., 2012), (Coss et al., 2012), (Sato et al., 2012), (Tappel et al., 2012a), (Wang et al., 2012), (Phithakrotchanakoon et al., 2013), (Tsai et al., 2013), (Vleeschouwer et al., 2014), (Tappel et al., 2014), (Sailer et al., 2015), (Galleano et al., 2016), (Hiroe et al., 2016), (Tappel et al., 2012b), and (Peprah et al., 2016). Alternatively, compounds of formula (I) can be obtained commercially, such as for example from: 007Chemicals BV, SH Deurne, The Netherlands; abcr GmbH, Karlsruhe, Germany; Aldlab Chemicals, LLC, Woburn, Mass., United States; Alfa Chemistry, Holtsville, N.Y., United States; Ark Pharm, Inc., Arlington Heights, Ill., United States; AstaTech, Inc., Bristol Pa., United States; Aurora Fine Chemicals LLC—USA, San Diego, Calif., United States; AURUM Pharmatech LLC, Franklin Park, N.J., United States; Key Organics Ltd, Camelford, United Kingdom; Carbosynth Limited, Compton, Berkshire, United Kingdom; CGeneTech, Inc., Indianapolis, Ind., United States; Clearsynth Canada Inc., Mississauga, ON, Canada; Combi-Blocks, Inc., San Diego, Calif., United States; Crysdot LLC, Baltimore, Md., United States; eNovation Chemicals LLC, Bridgewater, N.J., United States; Fluorochem Ltd., Hadfield, Derbyshire, United Kingdom; FUJIFILM Wako Pure Chemical Corporation, Osaka, Japan; Heteroz, LLC, Raleigh, N.C., United States; Manchester Organics, Cheshire, United Kingdom; Matreya LLC, State College, United States; Matrix Scientific, Columbia, United States; OXCHEM Corporation, Wood Dale, Ill., United States; Santa Cruz Biotechnology, Inc., Dallas, Tex., United States; Sigma-Aldrich/Merck, Darmstadt, Germany; Small Molecules, Inc., Hoboken, N.J., United States; Synnovator, Inc., Research Triangle Park, N.C., United States; Toronto Research Chemicals, North York, ON, Canada; Vijaya Pharmaceuticals, LLC (V-Pharma), Research Triangle Park, N.C., United States; Zerenex Molecular Ltd, Greater Manchester, United Kingdom.

Salt forms of the compound of formula (I) which may be formed, e.g., by protonation of an atom carrying an electron lone pair which is susceptible to protonation, such as an amino group, with an inorganic or organic acid, or as a salt of a carboxylic acid group with a suitable cation are well known in the art. Exemplary base addition salts comprise, for example, alkali metal salts such as sodium or potassium salts; alkaline-earth metal salts such as calcium or magnesium salts or ammonium salts. Exemplary acid addition salts comprise, for example, mineral acid salts such as hydrochloride, hydrobromide, hydroiodide, sulfate salts, nitrate salts, phosphate salts (such as, e.g., phosphate, hydrogenphosphate, or dihydrogenphosphate salts), carbonate salts, or hydrogencarbonate salts.

The compound of formula (I) can be present as a racemic mixture (also referred to herein as racemate), i.e. a 50:50 (mol %:mol %) mixture of the respective (S)-enantiomer and the respective (R)-enantiomer of the compound. Also encompassed herein are mixtures of different ratios of the (R)- and (S)-enantiomer of the respective compounds, such as e.g. ratios of 60:40, 70:30, 80:20, 90:10 or 95:5 (mol %:mol %), as well as all other numerical values in between these values that are not explicitly listed herein. Further encompassed herein is that the compound of formula (I) contains only a single enantiomer, i.e. that it is an enantiomerically pure or enantiopure compound. In those cases where the compound is not a racemate it is preferred that the (R)-enantiomer is present in higher amounts than the (S)-enantiomer. Most preferably, the compound is a pure (R)-enantiomer.

The compound of formula (I) can be used in combination with carriers and/or additives. Suitable carriers and additives are well known in the art and may e.g. be solid, semisolid or liquid compounds. Non-limiting examples of carriers include fillers, diluents, encapsulating material or formulation auxiliary of any type such as e.g. solvents, natural or regenerated mineral substances, thickeners, binders, pH adjusting compounds. Non-limiting examples of additives comprise tackifiers, emulsifiers, dispersants, wetting agents, micronutrient donors, fertilisers or other preparations that influence plant growth.

Preferred examples of solvents include aromatic hydrocarbons, preferably the fractions containing 8 to 12 carbon atoms, e.g. xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, ethylene glycol monomethyl or monoethyl ether, ketones such as cyclohexanone, strongly polar solvents such as N-methyl-2-pyrrolidone, dimethyl sulfoxide or dimethylformamide, as well as vegetable oils or epoxidized vegetable oils, such as epoxidized coconut oil or soybean oil; or water.

For dusts and dispersible powders, solid carriers are generally employed. Such solid carriers can be selected from e.g. natural mineral fillers such as calcite, talcum, kaolin, montmorillonite or attapulgite. Highly dispersed silicic acid or highly dispersed absorbent polymers may be added in order to improve the physical properties. Non-limiting examples for granulated adsorptive carriers are carriers of a porous type, for example pumice, sepiolite or bentonite; while non-limiting examples of non-adsorbent carriers include calcite or sand. Furthermore, pre-granulated materials of inorganic or organic nature can be used, e.g. dolomite or pulverised plant residues.

Examples of advantageous application-promoting additives also include e.g. natural or synthetic phospholipids of the series of the cephalins and lecithins.

Properties such as emulsifying, dispersing and wetting are influenced by the addition of surface-active compounds, or mixtures thereof, including non-ionic, cationic and/or anionic surfactants. Non-ionic surfactants include, without being limiting, polyglycol ether derivatives of aliphatic or cycloaliphatic alcohols, saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols. Non-limiting examples of cationic surfactants include quaternary ammonium salts which contain, as N-substituent, at least one C₈-C₂₂ alkyl radical and, as further substituents, un-substituted or halogenated lower alkyl, benzyl or hydroxy-lower alkyl radicals. Anionic surfactants can be selected from water-soluble soaps and water-soluble synthetic surface-active compounds. Suitable soaps are alkali metal salts, alkaline earth metal salts or un-substituted or substituted ammonium salts of higher fatty acids (C₁₀-C₂₂), e.g. the sodium or potassium salts of oleic or stearic acid or of natural fatty acid mixtures which can be obtained e.g. from coconut oil or tallow oil. Synthetic surfactants include, without being limiting, fatty alcohol sulfonates, fatty alcohol sulfates, sulfonated benzimidazole derivatives or alkylsulfonates.

Further additives may be selected from the group of binders, penetration enhancers, such as e.g. detergents, stabilizers, agents improving the odor of the composition, antifoaming agents, viscosity regulators, pH regulators and pH stabilizers.

Such a combination of the compound of formula (I) with carriers and/or additives can be prepared by e.g. homogeneously mixing the compound of formula (I) together with the carriers and/or additives, such as e.g. the solid or liquid carrier.

In accordance with the present invention, it is preferred that any additional compounds, such as the above described carriers and/or additives, are inert, i.e. they are not capable of inducing PTI. In other words, it is preferred in accordance with the methods of the present invention that the compound of formula (I) is the only ingredient in the composition capable of inducing PTI in plants. More preferably, any additional compound used in combination with the compound of formula (I) in any of the methods of the present invention is preferably a compound that does not exert a pathogen-protecting effect in plants. As described above, it is particularly preferred that no LPS and/or no lipid A is used in combination with the compound of formula (I) in any of the methods of the present invention. Most preferably, the compound of formula (I) is the only active ingredient.

Similarly, in as far as the methods of the present invention “comprise” additional method steps in addition to the specifically recited steps, it is preferred that such additional steps do not encompass the use of an additional compound capable of inducing PTI. More preferably, such additional steps do not encompass the use of an additional compound that exerts a pathogen-protecting effect in plants. Further preferred is that such additional steps do not encompass the use of LPS and/or lipid A. Most preferably, the compound of formula (I) is the only active ingredient used in any of the methods of the present invention.

In a second, subsequent step in accordance with the method of the invention, it is determined whether PTI is activated.

As described herein above, pattern triggered immunity, i.e. PTI, is a fundamental first layer of plant immunity and is well known in the art. Accordingly, numerous methods for the determination of activation of PTI are known in the art and have been described e.g. in (Ronald, 2006; Nguyen et al., 2010; McDowell, 2011; Lloyd et al., 2014; Lacomme, 2015; Schoonbeek et al., 2015; Botella and Botella, 2016; Duque, 2016; Aalen, 2017; Shan and He, 2017).

For example, transient increases in cytosolic calcium concentrations indicate that PTI has been activated. In order to determine transient increases in cytosolic calcium concentrations, a suitable reporter needs to be employed, typically a transgenic reporter such as aequorin as described e.g. in (Shimomura et al., 1962). This method has also been described in the appended examples (Example 1.4).

Activation of PTI is further accompanied by an accumulation of reactive oxygen species (ROS) or nitric oxide (NO). Means and methods to determine whether there is an accumulation of reactive oxygen species (ROS) or nitric oxide (NO) are well known in the art and have been described, e.g. in (Trujillo, 2016), (Zeidler et al., 2004) and (Lloyd et al., 2014) as well as in the appended examples (example 1.5).

PTI activation is further accompanied by an alkalinization of the cell culture medium, which can be determined by use of pH-sensitive electrodes as described, e. g. in (Felix et al., 1993).

The production of the plant hormones such as ethylene (ET), salicylic acid (SA) and/or jasmonic acid (JA) is another hallmark of PTI induction. Their production can be determined by high-performance liquid chromatography or gas chromatography as described, e. g. in (Vallarino and Osorio, 2016), (Beyer and Morgan, 1970), and (Deng et al., 2003).

Also the activation of mitogen-activated protein kinases (MAPKs) and/or calcium-dependent protein kinases (CDPKs) indicates the activation of PTI. Activation of these kinases can be determined for example by immunoblots analysis using a suitable antibody, as e.g. described in (Chung and Sheen, 2017) as well as in the appended examples (example 1.6) or by in-gel kinase assays as e.g. described in (Chung and Sheen, 2017) and (Seybold et al., 2017).

PTI activation is further characterized by the induction of defence gene expression, such as e.g. the expression of FRK1, NHL10, PHI1, WRKY33, PR1, PR2, PR4, PR5, or PDF1.2. Expression of defence genes can be detected by any method known in the art for gene expression analysis. Particularly preferred methods include qRT-PCR, as described e.g. in example 1.7 below, as well as promoter-β-glucuronidase (GUS) or promoter-luciferase (LUC) reporter assays as e.g. described in (Asai et al., 2002).

Activation of PTI is also accompanied by a fortification of cell walls, which can be determined by methods such as e.g. callose-staining (Jin and Mackey, 2017).

Systemic resistance is also induced in response to an activation of PTI. The induction of systemic resistance can be determined by methods well known in the art. To provide a non-limiting example, the compound to be employed can be infiltrated into specific leaves of a plant, which is subsequently infiltrated with a pathogen into different leaves. Pathogen growth in infected leaves is subsequently determined using methods such as microbial DNA quantification by real-time PCR (Pallas et al., 2009; Humphris et al., 2015) or bacterial enumeration (Katagiri et al., 2002). Such an approach has been described herein below in example 1.8.

Furthermore, immunoblot detection or activity-based detection of defense proteins can also be employed to detect the activation of PTI. To this end, proteins such as e.g. pathogenesis-related 1 (PR1) protein (e.g. (Huot et al., 2017) are typically detected, employing antibodies readily available in the art. Immunoblot detection of proteins is well known in the art and has been described, e.g. in (Liu et al., 2014). Also, activity-based detection of defense proteins can be employed to detect the activation of PTI, e.g. activation of peroxidases as described in (Mott et al., 2016).

Preferably, PTI is considered to be activated when, compared with an untreated control, at least one of the following changes is observed (i) a transient increase in cytosolic calcium concentrations; (ii) the accumulation of reactive oxygen species (ROS) or nitric oxide (NO); (iii) an alkalinization of the cell culture medium; (iv) the production of the plant hormones ethylene (ET), salicylic acid (SA) and/or jasmonic acid (JA); (v) the activation of mitogen-activated protein kinases (MAPKs) and/or calcium-dependent protein kinases (CDPKs); (vi) the induction of defense gene expression; (vii) cell wall fortification; (viii) induction of systemic resistance; and (ix) immunoblot detection of defense proteins. Most preferably, PTI is considered to be activated when, compared with an untreated control, at least the induction of systemic resistance (i.e. option (viii)) is changed. The term “comparison with an untreated control” includes, without being limiting, a comparison with the immunity status prior to treatment, or alternatively, a comparison with a sample that has been treated in the exactly same manner as the test sample, but with a compound that is known to not elicit PTI in the respective plants. A change is considered to be present when a statistical difference is observed. Means and methods for analyzing statistical significance are known in the art. As a non-limiting example, a change is considered to be significantly different from an untreated control when it differs for example by at least 2-, i.e. two-times the standard deviation of untreated control, i.e. for repeated determination of the control value. Alternatively, a change is considered to be significantly different if the PTI induced in the treated plants is at least 2-fold higher than in the untreated control, such as e.g. at least 5-fold higher, more preferably at least 10-fold higher.

It will be appreciated that in said control, these aspects can be determined prior to carrying out the method of the invention, or in parallel thereto. Furthermore, this step can be carried out once to provide a reference value for future use, or may be carried out each time the method is carried out.

Activation of PTI indicates that the plant expresses the functional LORE, or a functional variant thereof.

LPS has been considered to be a key player in plant-bacteria interactions, although the potentially underlying perception mechanisms have not yet been understood. The present inventors surprisingly found that LPS is not the necessary and sufficient compound required for triggering PTI in plants expressing the cell-surface receptor kinase LORE, but that PTI is instead triggered by medium chain 3-OH-fatty acids. Analysis by the inventors showed that typical LPS preparations—both commercial as well as freshly prepared preparations—that induced PTI in plants all contained medium chain 3-OH-fatty acids in free form. LPS preparations in which these medium chain 3-OH-fatty acids were removed did not trigger any responses in the standard early and late PTI assays, e.g. calcium, ROS and peroxidase assays, as shown in Example 6 below. Thus, these findings evidence that the preconception in the art about the role of LPS in plant immunity needs to be revised. This finding is surprising as even RP-HPLC purified lipid A samples previously reported in the art were now found to contain medium chain 3-OH-fatty acids as impurities, as shown in Table 1 below. As RP-HPLC is considered the gold standard for obtaining pure products, it was commonly accepted in the art that by using this approach, pure products were obtained and employed for further experimentation. There was never any assumption in the art that prior art compound preparations might contain an impurity, let alone that it is in fact this impurity that elicits the immune response in plants.

The present inventors demonstrated for the first time that medium chain-3-OH fatty acids are sensed in a chain-length- and hydroxylation-specific manner in plants and trigger typical PTI responses mediated by the cell-surface receptor kinase LORE. As medium chain-3-OH fatty acids are uncomplicated to produce and to employ, they represent a convenient and simple new tool for the activation of PTI in plants. In fact, medium chain-3-OH fatty acids provide numerous advantages. Contrary to LPS, they can be prepared synthetically and, thus, can be provided as a homogenous composition of the respective fatty acid in high purity. LPS, on the other hand, is typically extracted from bacterial cultures and contains a heterogenous mixture of various LPS variants. Even more: such LPS extracts typically contain further impurities which, by themselves, can be bioactive and can, thus, influence experimental results.

LPS is, furthermore, an amphiphilic compound which renders it difficult to solubilise in water or other solvents. Typically, LPS forms suspensions or micelles in aqueous solutions, in particular in high concentrations, which renders their use problematic. In addition, the formation of micelles can result in that the micelles get caught in the dense matrix of plant cell wall components. The medium chain-3-OH fatty acids employed in accordance with the present invention, on the other hand, are easily soluble in various organic solvents as well as in water.

The detergent-like characteristics of isolated LPS can, in addition, lead to unspecific side effects in biological test systems. Via its lipophilic Lipid-A domain, LPS can also integrate into biological membranes, thereby destroying or at least disturbing membrane integrity and leading to stress reactions.

None of these problems arise with small medium chain-3-OH fatty acids.

Thus, medium chain-3-OH fatty acids enable various lines of investigations and uses: (1) it can now be easily determined whether a plant expresses LORE in a functional manner, thereby delimiting those plants that can be protected against pathogens via PTI activation using medium chain-3-OH fatty acids; (2) plants can be screened to find further, slightly different variants of LORE, thereby broadening our knowledge about possible plant defence mechanisms as well as potentially identifying improved variants thereof; (3) LORE can be mutagenised and tested for loss-of-function and/or gain-of-function mutations, thereby enhancing knowledge about this receptor as well as potentially identifying improved variants thereof; and (4) the use of medium chain-3-OH fatty acids as a plant protective composition for plants perceptive therefore, such as e.g. plants naturally expressing the respective LORE or LORE variant or plants genetically engineered to express said receptor.

In a preferred embodiment of the method of the present invention, R₃ is —OH or —SH, and R₄ is ═O or ═S.

In an even more preferred embodiment of the method of the present invention, R₃ is —OH, and R₄ is ═O.

In another preferred embodiment of the method of the present invention, R₁ is selected from —OH, —OCH₃, —OCH₂CH₃, —O(CH₂)₂₋₃—CH₃, —SH, —NH₂, and —NH—CH₂—COOH.

More specifically, the compound of formula (I) according to the present invention is selected from (i)

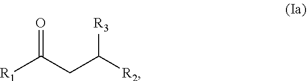

wherein
R₁ is selected from —OH, —OCH₃, —OCH₂CH₃, —O(CH₂)₂₋₃—CH₃, —SH, —NH₂, and —NH—CH₂—COOH;
R₂ is —(CH₂)₄₋₈—CH₃; and

### R₃ is —OH,

or
(ii) a compound of formula (Ib):

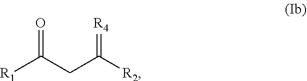

wherein
R₁ is selected from —OH, —OCH₃, —OCH₂CH₃, —O(CH₂)₂₋₃—CH₃, —SH, —NH₂, and —NH—CH₂—COOH;
R₂ is —(CH₂)₄₋₈—CH₃; and

### R₄ is ═O.

In yet another preferred embodiment of the method of the invention, the compound of formula (I) is selected from
3-hydroxydecanoic acid
3-hydroxyundecanoic acid
3-hydroxynonanoic acid
3-hydroxydodecanoic acid
3-hydroxyoctanoic acid
methyl-3-hydroxydecanoate
methyl-3-hydroxyundecanoate
methyl-3-hydroxynonanoate
methyl-3-hydroxydodecanoate
methyl-3-hydroxyoctanoate
ethyl-3-hydroxydecanoate
ethyl-3-hydroxyundecanoate
ethyl-3-hydroxynonanoate
(3-hydroxydecanoyl)glycine
(3-hydroxyundecanoyl)glycine
(3-hydroxynonanoyl)glycine
methyl (3-hydroxydecanoyl)glycinate
methyl (3-hydroxyundecanoyl)glycinate
methyl (3-hydroxynonanoyl)glycinate
3-oxo-decanoic acid
3-oxo-undecanoic acid
3-oxo-nonanoic acid
3-(hydroxydecanoyloxy)decanoic acid
3-(hydroxydecanoyloxy)dodecanoic acid
3-(hydroxydodecanoyloxy)dodecanoic acid
3-(hydroxyddecanoyloxy)decanoic acid
(3-hydroxydecanoyl)-L-leucine
propyl-3-hydroxydecanoic acid
butyl-3-hydroxydecanoic acid.

More preferably, the above recited preferred compounds of formula (I) are the (R)-enantiomer of the respective formula. Most preferably, the compound of formula (I) is the (R)-enantiomer of 3-hydroxydecanoic acid.

In another preferred embodiment of the method of the present invention, the plant(s) is/are contacted with the compound of formula (I) in a concentration of 1 nM to 1 mM.

More preferably, the amount of the compound of formula (I) is between 50 nM and 1 mM, more preferably between 500 nM and 500 μM, even more preferably between 1 μM and 250 μM and most preferably between 5 μM and 100 μM. Any numerical values not explicitly mentioned above but falling within the above recited preferred ranges are also envisaged herein. Alternatively, the application rate may be expressed as the amount of active ingredient per hectare to be treated. Preferably, the application rate is from 2 mg to 160 g of the compound of formula (I) per hectare, more preferably from 20 mg to 80 g of the compound of formula (I) per hectare, more preferably from 40 mg to 40 g and most preferably from 200 mg to 20 g of the compound of formula (I) per hectare.

The appropriate amount employed depends on the specific compound of formula (I) chosen and the intended method for contacting the plant(s) therewith and can be selected by the skilled person without further ado. For example, spraying onto leaves or pouring over roots often requires the use of higher concentrations than is required for direct application or infiltration, as a proportion of the sprayed/poured material does not reach the plant but instead is drained into the surrounding soil. Moreover, the amount chosen also depends on the application interval, with short-spaced intervals allowing for lower concentrations than intervals with long breaks in-between.

In a further preferred embodiment of the method of the invention, the functional variant of LORE is a naturally occurring or gene-technologically modified LORE mutant, a LORE-homologue or a LORE-analogue.

The term “gene-technologically modified LORE mutant”, as used herein, refers to the genetic engineering of the nucleic acid sequence encoding the LORE protein such that a LORE variant is generated whose amino acid sequence differs from the specifically recited amino acid sequence of SEQ ID NO:1 by a substitution, an inversion, an addition, an insertion and/or a deletion of one or several amino acids.

The term “substitution”, in accordance with the present invention, refers to the replacement of a particular amino acid with another amino acid. Thus, the total number of amino acids remains the same. In those cases where more than one amino acid is to be substituted, each amino acid is independently replaced with another amino acid, i.e. for each amino acid that is removed a different amino acid is introduced at the same position.

Substitutions, in accordance with the present invention, can be conservative amino acid substitutions or non-conservative amino acid substitutions.

The term “conservative amino acid substitution” is well known in the art and refers to the replacement of an amino acid with a different amino acid having similar structural and/or chemical properties. Such similarities include e.g. a similarity in polarity, charge, solubility, hydrophobicity, hydrophilicity, and/or the amphipathic nature of the residues involved. For example, nonpolar (hydrophobic) amino acids include alanine, valine, leucine, isoleucine, proline, phenylalanine, tyrosine, tryptophan, and methionine; polar neutral amino acids include glycine, serine, threonine, cysteine, asparagine, and glutamine; positively charged (basic) amino acids include arginine, lysine, and histidine; and negatively charged (acidic) amino acids include aspartic acid and glutamic acid. Non-conservative amino acid substitutions can be introduced in order to introduce new reactive groups. Preferably, in accordance with the present invention, the substitutions are conservative amino acid substitutions.

The term “inversion”, in accordance with the present invention, refers to a kind of mutation in which the order of the amino acids in a section of the amino acid sequence is reversed with respect to the remainder of the amino acid sequence.

The term “insertion”, in accordance with the present invention, refers to the addition of one or more amino acids to an amino acid sequence, wherein the addition is not to the C-terminal or N-terminal end of the amino acid sequence. In those cases where the addition is to the C-terminal or N-terminal end of the amino acid sequence, the mutation is referred to as “addition”.

The term “deletion”, as used in accordance with the present invention, refers to the loss of amino acids. It is well known in the art that functional polypeptides may be cleaved to yield fragments with unaltered or substantially unaltered function. Said number of amino acids to be removed may be one, two, three, four, five, six, seven, eight, nine, ten, 15, 20, 25, 30, 40, 50, 60, 70, or 80 or more than 80. Any other number between one and 80 is also deliberately envisaged. In particular, the removal of amino acids which preserve sequences and boundaries of any conserved functional domain(s) or subsequences in the sequence of the LORE protein are particularly envisaged. Means and methods for determining such domains are well known in the art and include experimental and bioinformatic means. Experimental means include the systematic generation of deletion mutants and their assessment in assays for activity known in the art and as described in the Examples enclosed herewith. Bioinformatic means include database searches. Suitable databases included protein sequence databases. In this case a multiple sequence alignment of significant hits is indicative of domain boundaries, wherein the domain(s) is/are comprised of the/those subsequences exhibiting an elevated level of sequence conservation as compared to the remainder of the sequence. Further suitable databases include databases of statistical models of conserved protein domains such as Pfam maintained by the Sanger Institute, UK (www.sanger.ac.uk/Software/Pfam).

Preferably, the gene-technologically modified LORE mutant is a LORE variant that is gene-technologically modified to contain gain-of-function mutations. Such LORE variants can, for example, be modified to be more sensitive and/or more specific with regard to the recognition of a compound of Formula (I), or to have a broader recognition pattern. Furthermore, such LORE variants can, for example, be modified to be more active, e.g. to have a stronger or longer lasting activity, for example by increasing protein stability.

Numerous methods for the gene-technological engineering of nucleic acid sequences, in particular molecular biology techniques, have been described in the art, e.g. in (Green, 2012), (Zhang et al., 2017), and (Yin et al., 2017). Preferred methods of engineering nucleic acid sequence include, without being limiting, random mutagenesis, site-directed mutagenesis, restriction-ligation cloning, Gibson assembly, Goldengate assembly, Goldenbraid, Infusion cloning, Gateway cloning, genome editing technologies including e.g. zinc-finger nucleases (ZFN), transcription activator-like effector nucleases (TALEN), and clustered regularly interspaced short palindromic repeats (CRISPR)-associated proteins (e.g. CRISPR-Cas9 or CRISPR-Cfp).

The term “LORE homologue”, as used herein, relates to LORE proteins that share a certain degree of sequence similarity with the LORE protein represented by SEQ ID NO:1 due to common ancestry. As such, the LORE homologue can be an orthologue or a paralogue. Preferably, the LORE homologue in accordance with the present invention has at least 60% sequence identity with SEQ ID NO:1, such as at least 70%, preferably at least 80%, more preferably at least 85%, even more preferably at least 90% and most preferably at least 95% sequence identity with SEQ ID NO:1. Known LORE homologues include, without being limiting, LORE found in Capsella rubella as well as in Eutrema halophilum (e.g. (Ranf et al., 2015).

The term “LORE analogue”, as used herein, relates to a protein that does not share a common ancestor with LORE represented by SEQ ID NO:1 and, accordingly, does not necessarily has a similar structure. Instead, such analogues share functional similarity with the LORE protein represented by SEQ ID NO:1. Such functional analogues can be identified, e.g., via their responsiveness to stimulation with a compound of formula (I) and the subsequent induction of PTI in plants expressing said functional analogue. Such LORE analogues can be of particular interest, due to the shared functionality as a mediator of PTI in plants.

In accordance with the present invention, any such variant of LORE needs to be a functional variant, as defined above.

In another further preferred embodiment of the method of the invention, the plant is a plant of the family Brassicaceae.

The family Brassicaceae is an economically relevant family of flowering plants commonly known as the mustards, the crucifers, or the cabbage family. Most are herbaceous plants, some shrubs, with simple, although sometimes deeply incised, alternatingly set leaves without stipules or in leaf rosettes, with terminal inflorescences without bracts, containing cruciform flowers with four sepals, four alternating petals, two short and four longer stamens, and a fruit with seeds in rows, divided by a thin wall or septum. The family contains the cruciferous vegetables, including species such as Brassica oleracea (e.g., broccoli, cabbage, cauliflower, kale, collards), Brassica rapa (turnip, chinese cabbage, etc.), Brassica napus (rapeseed, etc.), Brassica nigra (black mustard), Brassica juncea (brown mustard), Raphanus sativus (common radish), Armoracia rusticana (horseradish), Eruca sativa (Arugula), Eutrema japonicum (Wasabi) Lepidium sativum (garden cress), Sinapis alba (white mustard) but also a cut-flower Matthiola (stock) and the model organism Arabidopsis thaliana (thale cress). The family Brassicaceae, as well as the members of this family, is well known in the art and has been described e.g. in (Kadereit et al., 2014) and (A-Shehbaz et al., 2006) More preferably, the plant is a plant selected from Arabidopsis thaliana, Brassica spp. (e.g. Brassica oleracea, Brassica rapa, Brassica napus, Brassica nigra, Brassica juncea), Raphanus sativus, Eruca sativa, Eutrema japonicum, Armoracia rusticana, Lepidium sativum, and Sinapis alba.

The present invention further relates to a screening method for identifying functional variants of LORE represented by SEQ ID NO:1, capable of activating pattern-triggered immunity (PTI), wherein the method comprises the steps of: (a) determining whether one or more plant(s) express(es) LORE as represented by SEQ ID NO:1, or a functional variant thereof, by the method of the invention described above; (b) determining the amino acid sequence of the LORE or the functional variant thereof in the plants identified in (a); and (c) comparing the amino acid sequence determined in (b) with the amino acid sequence of LORE represented by SEQ ID NO:1, wherein any amino acid sequence that differs from the amino acid sequence of SEQ ID NO:1 encodes a functional variant of LORE. This method is also referred to herein as the “screening method of the invention”.

LORE as represented by SEQ ID NO:1 is a LORE receptor kinase originally found in Arabidopsis. Due to genetic variability between different plants, numerous variants of the LORE protein potentially exist. The present screening method thus aims at identifying such variants, with the proviso that they are functional, i.e. that they are capable of activating PTI.

To this end, it is determined in a first step whether PTI is activated in (a) plant(s) upon contacting with a compound of formula (I), thereby establishing whether said plant(s) express(es) LORE as represented by SEQ ID NO:1, or a functional variant thereof. This first step corresponds to the method of the invention defined herein above. Preferably, in this screening method of the invention, seedlings, leaves, leaf discs, and roots are contacted with the compound of formula (I).

If PTI is successfully activated in the first step, the amino acid sequence of the LORE or the functional variant thereof in these plants is determined in a second step (step (b)). To this end, polymerase chain reaction (PCR) with specific or degenerate primers, screening of DNA libraries, map-based cloning, genome walking, or RNA, whole genome, or exon capture sequencing can be employed (Peters et al., 2003; Alberts, 2017). The amino acid sequence is directly derivable form the established nucleic acid sequence.

Once the amino acid sequence of the LORE variant in the plant(s) under investigation has been identified, said sequence is compared to the amino acid sequences of SEQ ID NO:1. Any sequence found in (b) that differs from SEQ ID NO:1 represents a functional LORE variant capable of activating PTI.

All definitions and preferred embodiments provided herein above with regard to the method of determining whether a plant expresses LORE, such as the means and methods of detecting PTI activation, or preferred plant types and the like, apply mutatis mutandis to this screening method.

The present invention further relates to a method of inducing pattern-triggered immunity (PTI) in a plant, the method comprising: (a) contacting a plant that expresses LORE represented by SEQ ID NO:1, or a functional variant thereof capable of activating PTI, or a part of said plant, with a compound of formula (I):

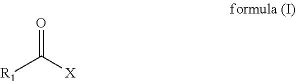

or a salt thereof,
wherein
R₁ is selected from —OH, —H, —OCH₃, —OCH₂CH₃,
—O—(CH₂)₂₋₁₁—CH₃, wherein one hydrogen atom in the —(CH₂)₂₋₁₁— group may be replaced by a —CH₃ group,
—O—(CH₂)₁₋₁₂—OH, wherein one or two hydrogen atoms in the —(CH₂)₁₋₁₂— group may be replaced by a —CH₃ group,
—O—(CH₂)₁₋₁₁—COOH, wherein one hydrogen atom in the —(CH₂)₁₋₁₁— group may be replaced by a —CH₃ group,
—NH₂, —SH, —SCH₃, —SCH₂CH₃,
an amino acid residue, preferably selected from Gly, Ala, Val, Leu, lie, Met, Thr, Ser, Cys, Gln, Asn, Glu, Asp, Arg or Lys, most preferably selected from Gly, Ala, Ser, Thr, Asp, Glu or Leu, which is attached via an amino group to form an amide bond with the carbonyl group of formula (I) and wherein the carboxyl group of the amino acid residue may be converted into an ester group, preferably a C₁-C₆ alkyl ester, more preferably a methyl ester, and a biogenic amine, preferably selected from putrescine, cadaverin, agmatine, spermidine or spermine, which is attached via an amino group to form an amide bond with the carbonyl group of formula (I);
and X is selected from one of formulas (II) and (III):

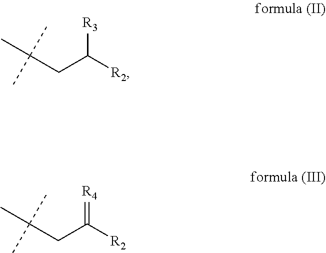

wherein
R₂ is —(CH₂)₄₋₈—CH₃;
R₃ is selected from —OH, —SH, —OCOCH₂CHOH(CH₂)₄₋₈CH₃; and
R₄ is selected from ═O, ═S, ═NH, and ═CH₂;
and the dashed line in formula (II) and (III) marks the bond which attaches X to the remainder of the compound of formula (I).

In accordance with the present invention, the compound of formula (I) is brought into contact with the plant in an amount and for a time sufficient to activate PTI. For example, the times and amounts employed in the appended examples may be applied. Preferably, the compound of formula (I) is brought into contact with the plant in a concentration of 100 nM to 1 mM for approx. 1 to 5 days.

This method is also referred to herein as the “PTI-induction method of the invention”.

As described herein above, the present inventors found that medium chain 3-OH-fatty acids can elicit plant immunity in plants expressing LORE or a functional variant thereof. Thus, also encompassed by the present invention is the use of said medium chain 3-OH-fatty acids in a PTI-induction method. All definitions and preferred embodiments provided herein above with regard to the method of determining whether a plant expresses LORE and the screening method of the invention, such as preferred compounds falling under formula (I) or preferred plant types and the like, apply mutatis mutandis to this PTI-induction method. Preferably, in this PTI-induction method of the invention, leave and/or roots are contacted with the compound of formula (I).

In a preferred embodiment of the PTI-induction method of the invention, the method further comprises the step: (a-0) modifying a plant to express LORE represented by SEQ ID NO:1, or a functional variant thereof capable of activating PTI.

This additional step in the PTI-induction method of the invention is to be implemented as the first step, i.e. prior to step (a) of contacting a plant that expresses LORE represented by SEQ ID NO:1, or a functional variant thereof capable of activating PTI, or a part of said plant, with a compound of formula (I).

Said modification of a plant can be carried out by means known in the art. To this end, the plant can be genetically engineered to express LORE represented by SEQ ID NO:1, or a functional variant thereof; or the plant can be crossed with another plant that already expresses LORE represented by SEQ ID NO:1, or a functional variant thereof, and selecting the resulting offspring for plants that express LORE represented by SEQ ID NO:1, or a functional variant thereof.

Inclusion of this additional step leads to the modification of plants that are not per se susceptible to activation of PTI by a compound of formula (I). This modification ensures that said plants become susceptible to a compound of formula (I) and, hence, that PTI can be induced in said plants. This modification can, alternatively, also serve to provide a plant with an additional or alternative variant of LORE, for example an improved variant of LORE, thereby increasing its capability to respond to pathogens. These plants are, thus, enabled—or better enabled—to protect themselves against pathogens upon contacting them with a compound of formula (I).

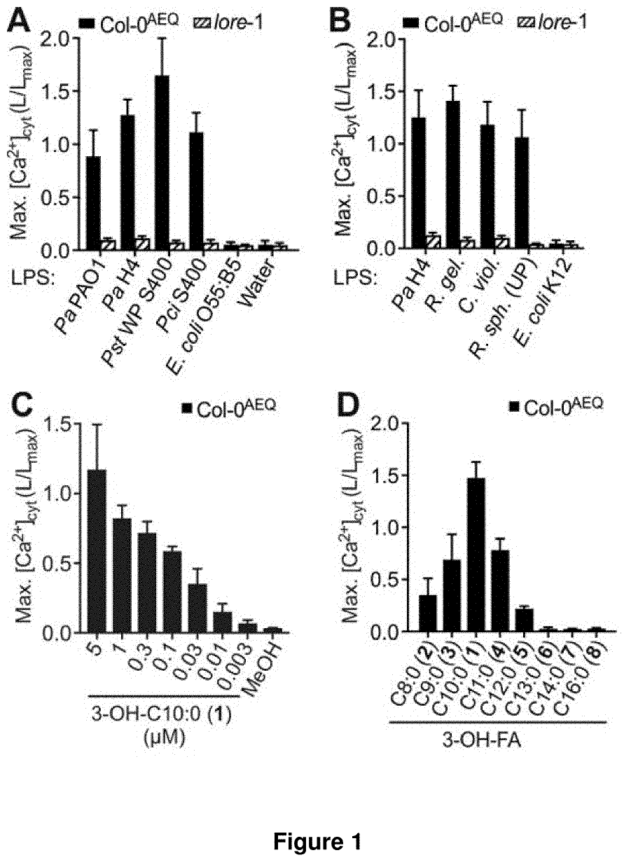

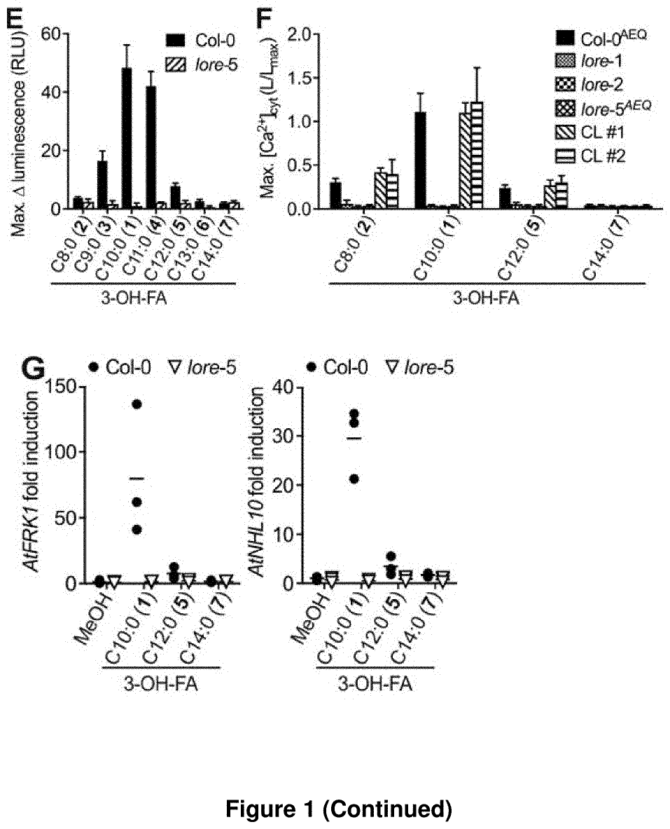

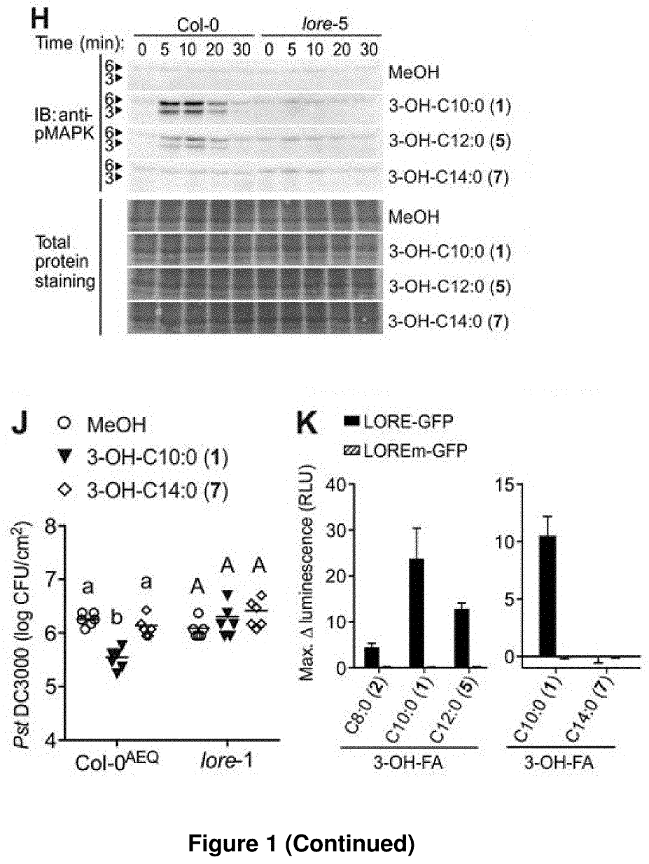

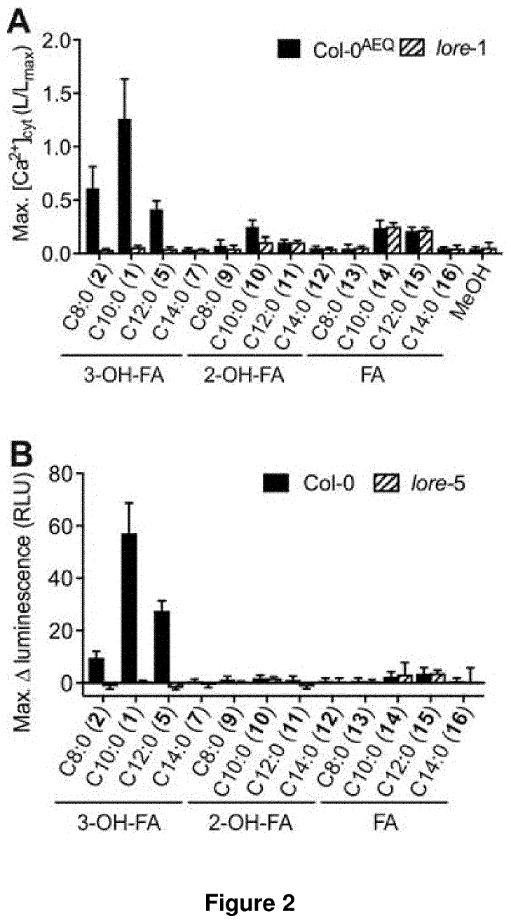

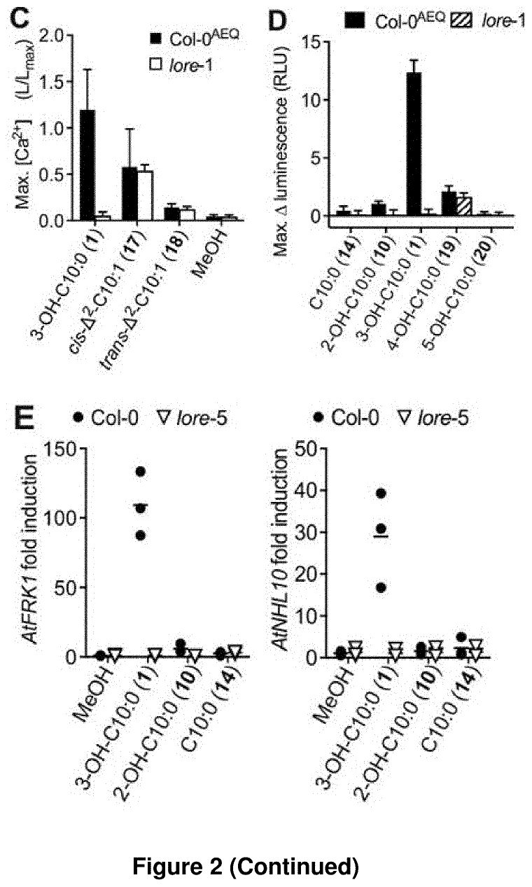

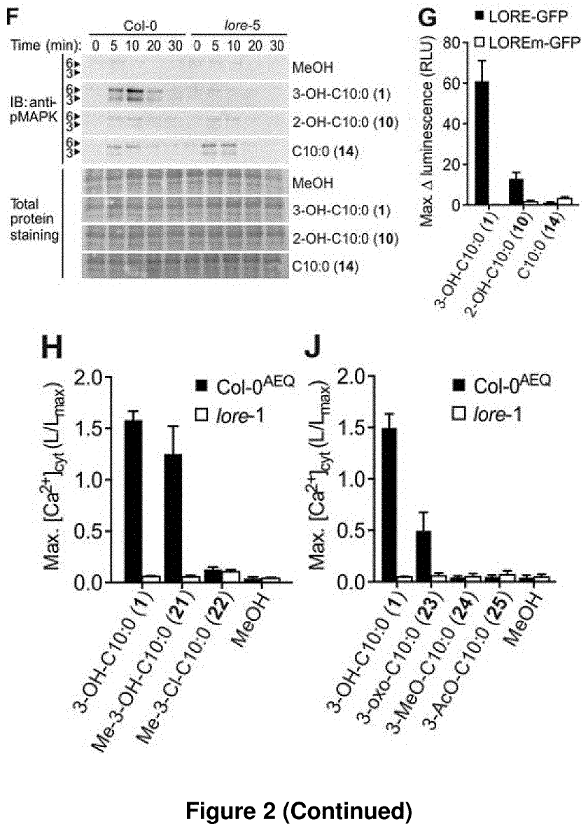

Successful modification of a plant to express LORE is shown in Examples 3 and 4 below, where solanaceous tobacco plants were modified to transiently express LORE-GFP in a gain-of-function approach (example 1.9), thereby rendering these plants responsive to medium chain 3-OH fatty acids, while control leaves expressing a kinase-inactive version (LOREm-GFP) show no medium chain 3-hydroxy fatty acids (mc-3-OH-FA)-induced response induced by medium chain 3-OH fatty acids (FIG. 1K, FIG. 2G). These findings show that a functional LORE is required and sufficient to render plants sensitive to medium-chain 3-OH fatty acids.

In a further preferred embodiment of the PTI-induction method of the invention, the functional variant of LORE capable of activating PTI is a functional variant of LORE identified by the screening method of the invention.

In a more preferred embodiment of the PTI-induction method of the invention, the modifying is by genetical engineering.

Means and methods for the genetical engineering of plants are well known in the art and include, without being limiting, Agrobacterium-mediated transformation, viral transformation, protoplast transformation, particle bombardment/biolistics, electro-transfection, microinjection, or DNA-free gene editing, as well as genome editing technologies including e.g. zinc-finger nucleases (ZFN), transcription activator-like effector nucleases (TALEN), and clustered regularly interspaced short palindromic repeats (CRISPR)-associated proteins (e.g. CRISPR-Cas9 or CRISPR-Cfp). Such methods have been described, e.g. in (Darbani et al., 2008), (Wilkinson, 2005), (Liang et al., 2018) as well as in the appended examples; e.g. Example 1.9. The modification can be such that expression is transient or stable. Preferably, the expression of LORE represented by SEQ ID NO:1, or a functional variant thereof, is stable expression.

The present invention further relates to a plant protective composition comprising or consisting of a compound of formula (I), as defined above, optionally in combination with carriers and/or additives. All definitions and preferred embodiments provided herein above with regard to the compound of formula (I) as well as with regard to suitable carriers and additives apply mutatis mutandis. In as far as the plant protective composition comprises (instead of consists of) a compound of formula (I), further active plant protective compounds may be included. Non-limiting examples of such active plant protective compounds include agents with fungicidal, bactericidal or virucidal activity or other compounds suitable to activate the plants' own defense system. Such compounds are well known in the art and examples for the first type of compound include, without being limiting, insecticides, fungicides, bactericides, nematicides, herbicides, molluscicides while examples for the second type of compound include, without being limiting, the chloronicotinyl or benzothiadiazole-derivates (e.g. US 2009/0018019 or U.S. Pat. No. 4,931,581) or mixtures of several of these active agents. Further examples of commonly employed active agents suitable for combination with the plant protective composition of the present invention include, without being limiting, tebuconazol, fludioxonil, metconazol, thiophanat-methyl, fluoxastrobin, prothioconazol, prochloraz, fluquinconazol, spiroxamine, difenoconazol, epoxiconazol, prothioconazol, triticonazol, dimoxystrobin, dimethoat, lambda-cyhalothrin, thiamethoxam, pirimiphos-methyl, metaflumizone, thiacloprid, beta-cyfluthrin, imidacloprid, spinosad, chlorantraniliprole, clothianidin, deltamethrin, diflubenzuron, spirodiclofen, alpha-cypermethrin, zeta-cypermethrin, boscalid, dimoxystrobin, metconazol, mepiquat or triadimenol.

Unless otherwise defined, all technical and scientific terms used herein have the same meaning as commonly understood by one of ordinary skill in the art to which this invention belongs. In case of conflict, the patent specification, including definitions, will prevail.

All the sequences accessible through the Database Accession Numbers cited herein are within the scope of the present invention and also include potential future updates in the database, in order to account for future corrections and modifications in the entries of the respective databases, which might occur due to the continuing progress of science.

All amino acid sequences provided herein are presented starting with the most N-terminal residue and ending with the most C-terminal residue (N→C), as customarily done in the art, and the one-letter or three-letter code abbreviations as used to identify amino acids throughout the present invention correspond to those commonly used for amino acids.

Regarding the embodiments characterised in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims. To give a non-limiting example, the combination of claims 10, 9 and 6 is clearly and unambiguously envisaged in view of the claim structure. The same applies for example to the combination of claims 12, 9 and 7, or the combination of claims 12, 9 and 4, etc.

### EXAMPLES

The following examples illustrate the invention:

### Example 1: Materials and Methods

1.1 Plant Material and Growth Conditions

A. thaliana Col-0 expressing pCaMV35S-apoaequorin in the cytosol (Col-0^{AEQ}) was obtained from M. Knight (Knight et al., 1991). Mutant lines lore-1, lore-2, complementation lines CL #1 and #2, and T-DNA insertion lines lore-5 (SAIL_857_E06, alias sd1-29), lore-5^{AEQ} (alias sd1-29^{AEQ}) were described previously (Ranf et al., 2015). For growth in liquid medium, surface-sterilized Arabidopsis seeds were stratified at 4° C. for >2 days and grown in liquid MS medium (0.5× Murashige & Skoog medium including vitamins (Duchefa), 0.25% sucrose, 1 mM MES, pH 5.7) in 24-well plates (˜15 seedlings/well) under long day (16 h light) conditions at 20° C./22° C. (night/day) (Ranf et al., 2012). Experimental Arabidopsis plants were grown on potting soil (ED73, Einheitserde) with vermiculite (5:1) in climate chambers under short day (8 h light) conditions at 20° C./22° C. (night/day), and N. benthamiana under long day (16 h light) conditions at 21° C./23° C. (night/day) in 55-60% relative humidity.

1.2 LPS Preparations and Repurifications

LPS preparations are summarized in table 1. Some LPS preparations were obtained from commercial suppliers or were kind donations (as indicated). Preparation of LPS from E. coli KPM 53 and purification of LA, core oligosaccharide and core-lipid A backbone oligosaccharide with amide-bound fatty acids (LPS-OH) of P. aeruginosa H4 was described previously (Ranf et al., 2015). Unpublished LPS and LA preparations and repurification of LPS preparations are described below.

Preparation and purification of LPS and LA of P. syringae pv. tomato DC3000. Bacteria were grown in King's B medium at 26° C. under shaking (230 rpm) to an absorbance of ˜1.0 at 600 nm, then they were harvested by centrifugation (3,000 g) for 20 min at 4° C., resuspended in a small volume of water, boiled for 45 min, and lyophilized. The bacterial mass was washed with ethanol, acetone (twice) and diethyl ether, and afterwards dried (recovery, 7.25 g). The pellet was resuspended in water (15 mg/ml), sequentially treated overnight at room temperature with DNase/RNase and proteinase K (each enzyme at 15 μg/ml), then underwent dialysis (14-kDa cutoff) and lyophilization. For hot phenol-water extraction (PW extraction) (Westphal and Jann, 1965), bacteria were resuspended in 45% aqueous phenol (10 ml per g bacteria) with an Ultra-Turrax and stirred for 20 min at 68° C. After centrifugation (5,500 g) for 20 min at 4° C., the upper water phase was collected. The extraction was repeated with the same volume of water. Combined water phases and the phenolic phase were dialyzed against water at 4° C. (14-kDa cutoff) and lyophilized. Prior to lyophilization, the dialyzed phenolic phase (PP) was centrifuged (600 g for 5 min at 20° C.) and divided into supernatant (sup) and sediment (sed). LPS recovered from the water phase (0.39 g; PstWP) was used as such, whereas a phenol-chloroform-petroleum ether extraction (Galanos et al., 1969) was performed with the material of the PP (1.27 g PP-sup; 1.54 g PP-sed). Each was resuspended in phenol (90%)-chloroform-petroleum ether (40-60° C.) (2:5:8 (vol/vol/vol); ˜80 mg/ml), then the respective suspension was stirred for 30 min at room temperature and centrifuged (5,800 g) for 20 min at 20° C. The supernatant was collected and the extraction was repeated twice. Combined supernatants were evaporated in vacuum until phenol crystallization began. LPS from PP-supernatant was precipitated overnight at 4° C. with 2 ml water and collected by centrifugation (7,650 g) for 30 min at 4° C. (Pst PP-sup sed1). To the supernatant, 12.5 ml ethanol were added, LPS was precipitated and collected by centrifugation (7,650 g for 30 min at 4° C.) (Pst PP-sup sed2). Since the supernatant after centrifugation was still turbid, another centrifugation (20,000 g for 1 h at 20° C.) was performed and the sediment (Pst PP-sup sed3) was collected. LPS from PP-sed was precipitated as described for PP-sup resulting in an aqueous precipitate (Pst PP-sed sed1) and an ethanol precipitate (Pst PP-sed sed2). All aqueous precipitates were washed twice with 80% phenol and three times with acetone, all ethanol precipitates only three times with acetone (centrifugation at 4,400 g for 20 min at 20° C.) and afterwards dried. Finally, this resulted in the following yields: Pst PP-sup sed1: 5.7 mg; Pst PP-sup sed2: 111.2 mg; Pst PP-sup sed3: 92.2 mg; Pst PP-sed1: 4.3 mg; Pst PP-sed2: 14.0 mg.

Preparation and purification of LPS and LA of P. cichorii ATCC10857/DSM50259. Bacteria were grown in King's B medium at 26° C. under shaking (230 rpm) to an absorbance of ˜1.0 at 600 nm, then were harvested by centrifugation (3,000 g) for 20 min at 4° C., resuspended in a small volume of water, boiled for 45 min, and lyophilized. The bacterial mass was washed with ethanol, acetone (twice) and diethyl ether, and afterwards dried (recovery, 13.5 g). For hot phenol-water extraction (PW extraction) (Westphal and Jann, 1965), bacteria were resuspended in 45% aqueous phenol (10 ml per g bacteria) with an Ultra-Turrax and stirred for 20 min at 68° C. After centrifugation (5,500 g) for 20 min at 4° C., the upper water phase was collected. The extraction was repeated with the same volume of water. The separately treated water phases (WP) and the phenolic phase (PP) were extensively dialyzed against water at 4° C. (14-kDa cutoff) and lyophilized. Prior to lyophilization, the dialyzed phenolic phase (PP) was centrifuged (600 g for 5 min at 20° C.) and divided into supernatant and sediment. All pellets were resuspended in water (10 mg/ml) and sequentially treated with DNase/RNase and proteinase K (each enzyme at 10 μg/ml), then underwent dialysis and lyophilization. This resulted in sediments of 0.23 g and 0.38 g for the water phases, respectively, and 0.59 g for the supernatant of the PP (Pci LPS), which was the LPS containing fraction.

Selected LPS preparations were further purified by gel permeation chromatography (GPC) on Sephacryl S-400 HR (GE Healthcare) on a column (2.5×120 cm) as described elsewhere (Jimenez-Barbero et al., 2002). Purification of 80 mg of Pci LPS in four runs d 20 mg resulted in 31 mg of purified LPS (Pci S400). Purification of 41.8 mg of Pst WP in two runs (21.8/20.0 mg) resulted in 17 mg of purified LPS (Pst WP-S400), 19.9 mg of Pst PP-sup sed2 in 8.7 mg of purified LPS (Pst PP-sup sed2-S400), and 19.8 mg of Pst PP-sup sed3 in 9.4 mg of purified LPS (Pst PP-sup sed3-S400).

Preparation and purification of Lipid A samples. Starting from LPS preparations Pst WP (335 mg) and Pci (404 mg), lipid A samples were basically generated as described earlier (Ranf et al., 2015). The following three modifications have been made: 1) heating for 3 h at 100° C. was done under reflux, 2) removal of SDS was achieved by six (Pst) or seven (Pci) washes with 120 ml 2 M HCl/ethanol (1:99 (vol/vol)), and 3) reextraction of the water phase was performed just twice with CHCl₃. Lipid A samples were further fractionated by reversed-phase HPLC essentially as described (Ranf et al., 2015), but with some modifications. A semi-preparative Kromasil C18 column (5 μm, 100 Å, 10×250 mm, MZ Analysentechnik) was used and the sample (10 mg/ml in CHCl₃/CH₃OH 4:1, v/v) was eluted using a gradient that consisted of methanol-chloroform-water (57:12:31, v/v/v) containing 10 mM NH₄OAc as mobile phase A and chloroform-methanol (70.2:29.8, v/v) with 50 mM NH₄OAc as mobile phase B. The initial solvent system consisted of 2% B and was maintained for 20 min, followed by a linear three step gradient raising from 2 to 17% B (20-50 min), 17 to 27% B (50-85 min), and 27 to 100% B (85-165 min). The solvent was held at 100% B for 10 min, the column re-equilibrated in 12 min to 2% B and held there for additional 10 min before the next injection. The flow rate was 2 ml/min using a splitter between the evaporative light-scattering detector (Sedex model 75C ELSD, S.E.D.E.R.E., France) equipped with alow-flow nebulizer recording the chromatogram and the fraction collector. Nitrogen (purity 99.996%) was used as gas to nebulize the post column flow stream at 3.5 bar into the detector at 50° C. setting the photomultiplier gain to 10. The detector signal was transferred to the Gilson HPLC Chemstation (Trilution LC, version 2.1, Gilson) for detection and integration of the ELSD signal.

Repurification of LPS from P. cichorii ATCC10857/DSM50259 by DOC-GPC. The S400-purified material of P. cichorii (Pci S400) was further fractionated on Sephacryl S-200 HR (GE Healthcare) on a column (1.5×120 cm) using a desoxycholate (DOC) containing buffer as described (Peterson and McGroarty, 1985). Approximately 5 mg LPS were resuspended in 1.5 ml eluting buffer and applied to the column. Material of two runs was combined in pools as indicated and lyophilized. Resulting sediments were washed four times with 30 ml ethanol (3,363 g (swing out rotor) for 10 min at 4° C.) and supernatants were discarded. Sediments were dried under a stream of nitrogen, stepwise dissolved in 9 ml water, transferred into dialysis tubes (12- to 16-kDa cutoff), and 1 ml 40 mM MgSO₄-solution was added. Dialysis against water was performed for three days at 4° C. with twelve water exchanges in total. Final yields starting from 10.4 mg Pci S400 were: Pci S400/S200 pool 1, 0.184 mg; Pci S400/S200 pool 2, 0.609 mg; Pci S400/S200 pool 3, 0.827 mg; Pci S400/S200 pool 4, 3.46 mg.

Heat-detergent-promoted repurification of LPS from P. aeruginosa H4. The heat-detergent-promoted repurification procedure was adapted from (Tirsoaga et al., 2007). Dried LPS preparations were resuspended in aqueous 1% (w/v) SDS solution (20 mg/mL LPS), incubated for 10 min at 100° C., and dispersed in an ultrasonic bath for 10 min. 12 mL chloroform and 8 mL methanol per ml SDS-LPS suspension were added. Suspensions were sonicated for 10 min and incubated overnight on a rotator at 4° C. Samples were centrifuged (10 min, 2000 g, 4° C.), the supernatant was discarded, and the pellet dried under a stream of nitrogen. This procedure was repeated four times using 1% (w/v) SDS solution and two times using Millipore-grade dH₂O (MP-dH₂O) to remove residual SDS. Finally, samples were washed 3 times with MP-dH₂O and suspended in MP-dH₂O.

1.3 Chemicals

Flg22 peptide was described previously (Gomez-Gomez et al., 1999). Synthetic compounds were obtained from commercial suppliers or generated herein as indicated in table 2.

1.4 Aequorin Luminescence Measurements

Aequorin luminescence measurements were performed essentially as described (Ranf et al., 2012). In short, 8- to 10-days-old liquid-grown apoaequorin-expressing seedlings were placed individually in 96-well plates in MP-dH₂O containing 5-10 μM coelenterazine-h (p.j.k. GmbH) in the dark overnight. Luminescence was recorded by scanning 2 rows in 10 sec intervals using a Luminoskan Ascent 2.1 (Thermo Scientific). Remaining aequorin was discharged by addition of 150 μl 2 M CaCl₂) with 20% EtOH per well. [Ca²⁺]_{cyt} concentrations were calculated as L/Lₘₐₓ (luminescence counts per sec/total luminescence counts remaining).

1.5 ROS Detection in Arabidopsis Leaves

ROS production was monitored as described (Ranf et al., 2015) in 3 mm leaf discs from 6-8 weeks-old soil-grown plants in 100 μl 5 μM L-012 (WAKO chemicals) and 2 μg/ml horseradish peroxidase (Type II, Roche) in MP-dH₂O. For experiments including 4-OH-10:0 (19) and 5-OH-10:0 (20), 10 mM Tris pH 8 was added to the L-012/HRP mix. Luminescence was recorded as relative light units (RLU) in 1 min intervals using a Luminoskan Ascent 2.1 (Thermo Scientific) or a Tecan F200. After 10 min background reading, elicitors were added to the final concentrations indicated and luminescence readings continued over 45-60 min. Data are depicted after normalization to average ROS levels 5 min before elicitor application and subtraction of water or MeOH controls that were included for each genotype on the same plate.

1.6 Immunoblot Analysis of MAPK Activation

MAPK activation was elicited in 14-days-old liquid-grown seedlings as described (Ranf et al., 2015) by adding MAMPs to the final concentrations indicated. Seedlings were harvested at the indicated time points, frozen in liquid nitrogen and homogenized using a bead mill (TissueLyser II, Qiagen). Proteins were extracted in kinase extraction buffer containing 50 mM Tris/HCl pH 7.5, 100 mM NaCl, 20 mM EGTA, 30 mM beta-glycerophosphate, 30 mM 4-p-Nitrophenylphosphate, 4 mM NaF, 4 mM Na₃VO₄, 4 mM Na₂MoO₄, 10 mM DTT, 0.2% Tween 20, and 1× plant protease inhibitor cocktail P9599 (Sigma-Aldrich). 50 ag total protein (according to Bradford Protein Assay, Bio-Rad) was separated by standard SDS polyacrylamide gel-electrophoresis (Mini-PROTEAN Tetra Cell, Bio-Rad) and blotted onto nitrocellulose membrane (Amersham™ Protran™ 0.2 μm NC, GE Healthcare) using a Trans-Blot® SD Semi-Dry Transfer Cell (Bio-Rad). Membranes were blocked in Protein-Free TBS Blocking Buffer (Pierce™) and immunostained with anti-phospho-MPK antibody (anti-phospho-p44/42-ERK, 1:1000, Cell Signaling Technology #9101) and anti-rabbit-IgG-peroxidase conjugate (A9169, 1:50000, Sigma-Aldrich) using SuperSignal™ West Dura Extended Duration Substrate (Thermo Scientific). ECL chemiluminescence was detected using a Fusion SL camera (Vilber Lourmat). Membranes were stained with Amido black to assess equal protein loading.

1.7 Gene Expression Analysis

12-days-old liquid-grown seedlings were equilibrated in fresh MS medium for two days before treatment with 1 μM of the indicated elicitors or the respective amount of MeOH as control in MS medium. Seedlings were harvested at four hours, frozen in liquid nitrogen and homogenized using a bead mill (TissueLyser II, Qiagen). Total RNA was extracted using the conventional Trizol method. 2 μg total RNA was digested with DNaseI (Thermo Scientific) and reverse-transcribed with oligo(dT), and Revert Aid® reverse transcriptase (Thermo Scientific) according to the manufacturers' instructions. Gene expression was determined by quantitative PCR with primers described previously for AtFRK1 (He et al., 2006), AtNHL10 (Boudsocq et al., 2010) and AtUBQ5 (Weis et al., 2013). Quantitative real-time PCR was performed on a AriaMx Real-time PCR System G8830A (Agilent Technologies Inc.) according to manufacturer's instructions (3 min at 95° C., 40 cycles of 5 sec at 95° C., 20 sec at 60° C., and 20 sec at 72° C.) using the Maxima SYBR Green-ROX qPCR Master Mix (Thermo Scientific; 1 μl of 1:10 diluted cDNA in 10 μl reaction volume) and analysed using the AriaMX Software V1.3 (Agilent). Primer efficiencies were determined according to manufacturer's instructions and found to be close to 100%. Amplification specificity was analysed by non-template controls and primer dissociation curves. Biological replicate samples were analysed in triplicates side-by-side in one run for each gene. Expression levels of AtFRK1 and AtNHL10 were normalized to AtUBQ5 expression levels and fold changes relatively to the expression level of the Col-0 MeOH control calculated by the 2^{-ΔΔCt} method as described (Livak and Schmittgen, 2001).

1.8 Induced Resistance Assay

10 μM 3-OH-FAs or the respective amount of MeOH as control were syringe infiltrated into the first two true leaves of 4- to 5-week-old Arabidopsis plants as described (Breitenbach et al., 2014). After two or three days Pst DC3000 was syringe infiltrated into leaves 4 and 5 of the pretreated plants at 1×10⁵ CFU/ml. Bacterial growth in infected leaves was determined 4 days later as described (Vlot et al., 2008). Statistical analysis (one-way analysis of variance, ANOVA, with Tukey's multiple comparison post-test) was applied separately for wild type and mutant plants using GraphPad Prism 7.0.

1.9 Transient Expression in Tobacco

Overnight cultures of Agrobacterium tumefaciens GV3101 carrying LORE-GFP or kinase-inactive LOREm-GFP (K516A) constructs (Ranf et al., 2015) or the p19 suppressor of silencing (Voinnet et al., 2003) were grown in AB medium supplemented with 100 μM acetosyringone (Wise et al., 2006), harvested by centrifugation (1,000 g for 5 min) and washed twice with infiltration medium (10 mM MgCl₂, 10 mM MES pH 5.7, 150 μM acetosyringone). Bacteria were resuspended in infiltration medium to an OD₆₀₀ of 0.05 and incubated at room temperature for 4-6 h. Bacteria carrying LORE-GFP or LOREm-GFP constructs were each mixed 1:1 with p19 cultures and syringe infiltrated into halves of the same leaves of 6-8 weeks-old N. benthamiana plants. Leaf discs (3 mm) were cut and used for ROS detection two days after infiltration as described above.

1.10 Quantification of 3-OH—C10:0 (1) by SIDA-UHPLC-MS/MS

3-Hydroxy decanoic acid (1) was analyzed by means of the newly developed stable isotope dilution analysis (SIDA-UHPLC-MS/MS). To this end, its deuterium-labeled twin molecule 3-OH—C10:0-d₂ (1-d₂) was synthesized.

Internal Standard (IS).

A stock solution (20 mL) of the 3-OH—C10:0-d₂ (1-d₂) (5 mmol/L, 3.8 mg/20 mL) was prepared in MeOD, its exact concentration was verified by means of quantitative NMR (qNMR) and it was stored at −20° C. until use.

Sample Preparation.

Samples were solved in water, acetonitrile or a mixture of acetonitrile/water. After adding 2 μL of the IS and the use of a Vortexer (2 min, 250 UPM, VWR, Darmstadt, Germany), samples were equilibrated for one hour and were shaken again (2 min, 250 UPM).

Ultra High Performance Liquid Chromatography-Mass Spectrometry (UHPLC-MS/MS).

A QTRAP 6500 mass spectrometer (Sciex, Darmstadt, Germany) was used and operated in the full-scan mode (ion spray voltage, −4500 V): curtain gas, 35 psi; temperature, 500° C.; gas 1, 55 psi; gas 2, 65 psi; collision-activated dissociation, −3 V; and entrance potential, −10 V. For tuning direct flow injection with a syringe pump (10 μL/min) and compound solutions in ACN/water were used. The samples were separated by means of a Nexera X2 UHPLC (Shimadzu Europa GmbH, Duisburg, Germany) consisting of two LC pump systems 30AD, a DGU-20A5 degasser, a SIL-30AC autosampler, a CTO-30A column oven, and a CBM-20A controller and equipped with a 100×2.1 mm, 100 Å, Kinetex 1.7 μm C18 column (Phenomenex). Chromatography was performed with an injection volume of 2 μL and a flow rate of 0.4 mL/min. The solvent system consisted of A: acetonitril (0.1% formic acid) and B: formic acid (0.1% in water, pH 3.5). The following gradient was used: 0 min, 30% A; 1 min, 30% A; 5 min, 100% A; 6 min, 100% A; 8 min, 30% A; 10 min, 30% A. Data acquisition and instrumental control was performed with Analyst 1.6.2 software (Sciex, Darmstadt, Germany). After optimizing instrument settings, analytes and the internal standard were analysed using the MRM transition Q1/Q3 of m/z 187.1/58.3 as qualifier (DP=−80 V, CE=−16 V, CXP=−7 V) and Q1/Q3 of m/z 187.1/187.1 as quantifier (DP=−80 V, CE=−8 V, CXP=−19 V) for 3-hydroxy decanoic acid (1) and Q1/Q3 of m/z 189.0/58.9 as qualifier (DP=−55 V, CE=−16 V, CXP=−9 V) and Q1/Q3 of m/z 189.0/189.1 as quantifier (DP=−55 V, CE=−8 V, CXP=−13 V) for 3-OH—C10:0-d₂ (1-d₂).

Calibration Curve and Linear Range.

A stock solution of purified 3-OH—C10:0-d₂ (IS, 1-d₂) and the analyte 3-OH—C10:0 (1) was prepared in MeOD, and its exact concentration was verified by means of quantitative NMR (qNMR). Thereafter, the IS (1-d₂) and the analyte (1) were mixed in 10 molar ratios from 0.05 to 50 keeping a constant concentration of the internal standard. Triplicate UHPLC-MS/MS analysis calibration curves were prepared by plotting peak area ratios of each analyte to the internal standard against concentration ratios of each analyte to the IS using linear regression, showing linear responses with correlation coefficients of >0.99 each. The response was linear for chosen molar ratios and the contents of 3-OH—C10:0 (1) in the samples was calculated using the respective calibration function. Determination of the limit of detection (LOD) at a signal-to-noise ratio of 3 and the limit of quantitation (LOQ) at a signal-to-noise ratio of 10 revealed the following values: LOD: ≤50.002 μM; LOQ≤0.01 μM.

1.11 Long-Term ROS Detection in Arabidopsis Leaves

Long-term ROS production was monitored in 3 mm leaf discs from 6-8 weeks-old soil-grown plants in 200 μL 200 μM luminol (luminol sodium salt, Sigma-Aldrich) and 10 μg/mL horseradish peroxidase (Type II, Roche) similarly as described (Shang-Guan et al., 2018). Luminescence was recorded as relative light units (RLU) using a Luminoskan Ascent 2.1 (Thermo Scientific) or a Tecan F200 luminometer. After 10 min background reading in 1 min intervals, elicitors were added to the final concentrations indicated and luminescence readings continued for 45-60 min in 1 min intervals and for further 40 h in 5 min intervals. Data are depicted after normalization to average ROS levels 5 min before elicitor application and subtraction of water or MeOH controls that were included for each genotype on the same plate.

1.12 Detection of Peroxidase Activity in Arabidopsis Leaves

Peroxidase secretion and activity was monitored as described (Mott et al., 2018) with minor modifications. Briefly, 3 mm leaf discs from 8-10 weeks-old soil-grown plants were incubated in 200 μL 0.5×MS medium for 1 h. Medium was replaced by 60 μL elicitor solutions (diluted in 0.5×MS medium) and incubated for 24 h in the dark. 50 μL were withdrawn and mixed with 50 μL 5-aminosalicylic acid solution (1 mg/mL, pH 6.0, Sigma-Aldrich) with 0.01% hydrogen peroxide. The reaction was stopped by adding 20 μL 2 M sodium hydroxide solution and absorbance at 600 nm was assessed. Fold change was calculated and statistically analysed using two-way ANOVA with Tukey's post-hoc test (confidence level 0.95, letter threshold p<0.05) after normal distribution of the data was confirmed by Shapiro-Wilk normality test.

1.13 Genetic Knockout of pagL in P. syringae pv. Tomato DC3000

Knockout plasmids were constructed using a golden gate-compatible pGGKO-blue plasmid derived from pK18mobsacB (Kvitko and Collmer, 2011). Flanking sequences (flank A: 550 bp, flank B: 553 bp) up and downstream of pagL (PSPTO_5636) were PCR-amplified from genomic DNA of P. syringae pv. tomato DC3000 (Pst) and inserted into the pGGKO-blue backbone by golden gate cloning using Bpil. A gentamicin resistance cassette (Gm^{R}) was amplified from plasmid pPS856 (Hoang et al., 1998) and inserted between the flanking sequences (flank A forward primer 5′-TTTGAAGACTGTCGAGCCCTCAGATTCGTCAAC-3′ (SEQ ID NO:3) and reverse primer 5′-TTTGAAGACGCGGCCGCCATGGTGGATTCGCCGGT-3′ (SEQ ID NO:4); flank B forward primer 5′-TTTGAAGACGCGGCCGCTGATTTGACTGGCACTTGTGC-3′ (SEQ ID NO:5) and reverse primer 5′-TTTGAAGACGTCTAGGGAAGTGATGCTTATCACCG-3′ (SEQ ID NO:6)). Mutants of Pstwere generated similar to the method described by (Kvitko and Collmer, 2011). Briefly, knockout plasmids were transferred to Pstvia triparental mating with E. coli HB101 (carrying plasmid pRK2013) as helper strain and E. coliDH5a (carrying the knockout plasmid) as donor. Sucrose counter selection was used to screen cells which underwent complete homologous recombination replacing the target gene with the gentamicin resistance cassette. Mutants were verified by PCR and sequencing of the amplicon (forward primer 5-GGGCTGGTCGAGCTGATCGAG-3′ (SEQ ID NO:7), reverse primer 5-TGCTCGACCTGCGCAGC-3′ (SEQ ID NO:8)).

1.14 Preparation and Purification of LPS of P. syringae pv. Tomato DC3000 ΔpagL::Gm^{R}

Bacteria were grown in King's B medium at 26° C. under shaking (230 rpm) to an absorbance of ˜1.0 at 600 nm, harvested by centrifugation (3,000 g) for 20 min at 4° C., washed three times with water, resuspended in a small volume of ethanol, and stirred at room temperature overnight. The suspension was filtrated and collected cells were washed with ethanol, acetone (twice), and diethyl ether, and dried (recovery, 11.7 g). The pellet was resuspended in water (˜15 mg/mL) with 0.02% NaN₃, sequentially treated overnight at room temperature with DNase/RNase and proteinase K (100 μL of 10 mg/mL solutions per gram dry weight for each enzyme), then underwent dialysis (14-kDa cutoff) and lyophilization. For PW extraction (Westphal and Jann, 1965), bacteria were resuspended in 45% aqueous phenol (10 mL per g bacteria) and stirred for 30 min at 68° C. After centrifugation (5,600 g) for 20 min at 4° C., the upper water phase was collected. The extraction was repeated with the same volume of water as had been collected. Combined water phases and the phenolic phase were dialyzed against deionized water at room temperature (14-kDa cut-off) and lyophilized. Prior to lyophilization, the dialyzed phenolic phase (PP) was centrifuged (600 g for 5 min at 20° C.) and divided into supernatant (sup) and sediment (sed). LPS recovered from the water phase (77.4 mg; Pst ΔpagL WP) was used as such, whereas a phenol-chloroform-petroleum ether extraction (Galanos et al., 1969) was performed with the material of the PP (4.06 g PP-sup; 1.42 g PP-sed). Each was resuspended in phenol (90%, w/v)-chloroform-petroleum ether (2:5:8 (vol/vol/vol); ˜0.1 mg/mL), then the respective suspension was stirred for 30 min at room temperature, and centrifuged (6,000 g) for 20 min at 20° C. The supernatant was collected and the extraction was repeated once. Combined supernatants were evaporated in vacuum until phenol crystallization began (at room temperature and normal pressure). Lipopolysaccharide from PP-sup was precipitated for 4 days at 4° C. with 1.37 mL water and collected by centrifugation (4,400 g) for 20 min at 20° C. LPS from PP-sed was precipitated with 1.60 mL water as described for PP-sup. Precipitates were washed twice with 80% phenol (4,400 g, 20° C., 20 min) and three times with acetone (4,400 g, 20° C., 20 min) and dried. This resulted in the following yields: Pst ΔpagL PP-sup sed: 5.3 mg; Pst ΔpagL PP-sed sed: 58.9 mg.

### Example 2: Structural Features of LA that Mediate its Recognition

To decipher the structural features of LA relevant to its recognition in Arabidopsis, LPS with different LA acylation patterns were screened for activation of cytosolic calcium ([Ca²⁺]_{cyt}) signalling as an indicator of a PTI response in Arabidopsis. LPS of Pseudomonas syringae pv tomato (Pst) DC3000 contained predominantly hexa-acylated LA, whereas Pa strains PAO1 (Lam et al., 2011) and H4 (Ranf et al., 2015), and Pseudomonas cichorii (Pci) produced mainly penta-acylated LPS, lacking the 3-hydroxydecanoyl chain at the position C-3 of LA. All LPS samples triggered similar levels of LORE-dependent [Ca²⁺]_{cyt} signalling in Arabidopsis (FIG. 1A) (Ranf et al., 2015). LPS preparations from the phylogenetically distant species Rhodobacter sphaeroides, Chromobacterium violaceum and Rubrivivax gelatinosus, which share a similar LA acylation pattern with Pseudomonas LPS (Wollenweber et al., 1984; Masoud et al., 1990; Alexander and Rietschel, 2001), also activated LORE-dependent [Ca²⁺]_{cyt} signalling with comparable amplitude and kinetics to Pa H4 LPS (FIG. 1B). Structurally, all tested LPS that elicited LORE-dependent responses comprise a comparably short acyl chain, mostly 3-OH-decanoyl, at position C-3 and/or C-3′ of LA. LPS from E. coli, S. enterica, or B. cepacia with 3-OH-tetradecanoyl chains at these positions was inactive (FIG. 1A,B) (Raetz and Whitfield, 2002; Molinaro et al., 2009; Ranf et al., 2015). This correlation suggested that a 3-OH-decanoyl chain is a key structural feature for LORE-dependent LPS/LA immune sensing in Arabidopsis.

### Example 3: PTI Induction by Free Synthetic Medium Chain 3-Hydroxyfatty Acids (Mc-3-OH-FAs)

Therefore, synthetic 3-OH-FAs of varying chain length (Table 2) were tested for their ability to induce typical PTI responses in Arabidopsis. Strikingly, free synthetic 3-OH—C10:0 (1) was sufficient to elicit [Ca²⁺]_{cyt} elevations with comparable characteristics as Pseudomonas LPS in Arabidopsis at concentrations in the nanomolar to micromolar range (FIG. 1C). Notably, only medium chain 3-hydroxy fatty acids (mc-3-OH-FAs) with 8 to 12 carbon atoms (1-5) activated [Ca²⁺]_{cyt} signalling and ROS production, whereas long chain (Ic) 3-OH-FAs (13 to 16 carbon atoms, 6-8) did not (FIG. 1D-E).

Mc-3-OH-FA sensing was completely abolished in lore mutant lines but restored to levels comparable to the wild-type control upon genetic complementation of the lore-1 mutant with a genomic DNA fragment covering the LORE open reading frame and a 1-kilobase upstream cis-regulatory region (FIG. 1F) (Ranf et al., 2015).

Mc-3-OH-FAs, particularly 3-OH—C10:0 (1), also induced transcript accumulation of typical PTI response genes, AtFRK1 and AtNHL10, and phosphorylation of mitogen-activated protein kinases (MAPK), AtMPK3 and AtMPK6, whereas Ic-3-OH-FAs did not induce these responses (FIG. 1G-H). In plants, local application of MAMPs results in systemic resistance to pathogen infection in distal, untreated tissues (Mishina and Zeier, 2007). In line with the observed activation of LORE-dependent PTI signalling upon 3-OH—C10:0 (1) application (FIG. 1C-H), wild-type Arabidopsis plants pre-treated with 3-OH—C10:0 (1) were more resistant to subsequent infection with virulent Pst DC3000 by leaf infiltration, but this systemic resistance was not activated in Arabidopsis lore-1 mutant plants (FIG. 1J). By contrast, Arabidopsis plants pre-treated with 3-OH—C14:0 (7), which did not induce PTI signalling (FIG. 1D-H), did not establish systemic resistance to bacterial infection (FIG. 1J).

The solanaceous plant species Nicotiana benthamiana is insensitive to Pseudomonas LPS but gains responsiveness upon transient expression of a functional LORE-GFP (Ranf et al., 2015). Mc-3-OH-FAs also induced ROS production upon expression of a functional LORE-GFP fusion in N. benthamiana leaves, but not in control leaves expressing a kinase-inactive LORE variant with a mutated ATP-binding site in the kinase domain, whereas Ic-3-OH-FAs did not trigger a response (FIG. 1K). Taken together, using synthetic compounds it was demonstrated that in Arabidopsis 3-OH-FAs are sensed as elicitors in a chain-length dependent manner: only mc-3-OH-FAs—with an optimum chain length of 10 carbon atoms—activate PTI, whereas Ic-3-OH-FAs do not. Furthermore, LORE is pivotal for chain-length dependent sensing of 3-OH-FAs.

### Example 4: Structural Requirements for LORE-Mediated PTI Activation

Next, it was tested whether the position of the hydroxyl group is vital for immune sensing of mc-3-OH-FAs in Arabidopsis (FIG. 2A-F). Compared to mc-3-OH-FAs (1-5), mc-2-OH-FAs (9-11) and non-hydroxylated mc-FAs (13-15) induced only limited PTI responses such as [Ca²⁺]_{cyt} signalling, ROS production, defence gene induction, and MAPK activation in Arabidopsis or ROS production in LORE-GFP-expressing N. benthamiana (FIG. 2A-G). Moreover, PTI responses induced by non-hydroxylated or Δ²-unsaturated (17,18) mc-FAs were weak and independent of LORE (FIG. 2A-F). 4-OH—C10:0 (19) and 5-OH—C10:0 (20) also did not elicit LORE-dependent ROS production (FIG. 2D).

Substitution of the hydroxyl group at position 3 of 3-OH—C10:0 with chlorine (22) or its modification with a methyl ether (24) or an acetyl ester (25) abolished elicitor activity (FIG. 2H-J). By contrast, 3-oxodecanoic acid (3-oxo-C10:0, 23) or its keto-enol tautomer 3-OH-decenoic acid, which spontaneously forms in aqueous solution, induced a moderate LORE-dependent [Ca²⁺]_{cyt} response (FIG. 2J).

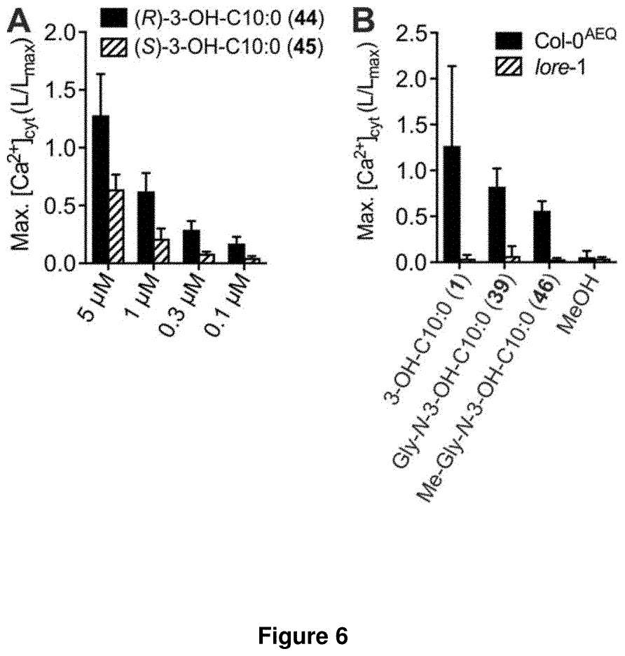

The synthetic 3-OH-FAs used here are racemates, but Pseudomonas LPS comprises typically (R)-3-OH-decanoyl as primary ester-linked acyl chains (Zähringer et al., 1994). (R)-3-OH—C10:0 (44) induced stronger immune signalling than (S)-3-OH—C10:0 (45) (FIG. 6A).

Collectively, these data suggest that, in combination with a medium acyl chain length, a free 3-hydroxyl group is essential for triggering LORE-dependent immune signalling in Arabidopsis.

### Example 5: Further Structural Requirements for LORE-Mediated PTI Activation

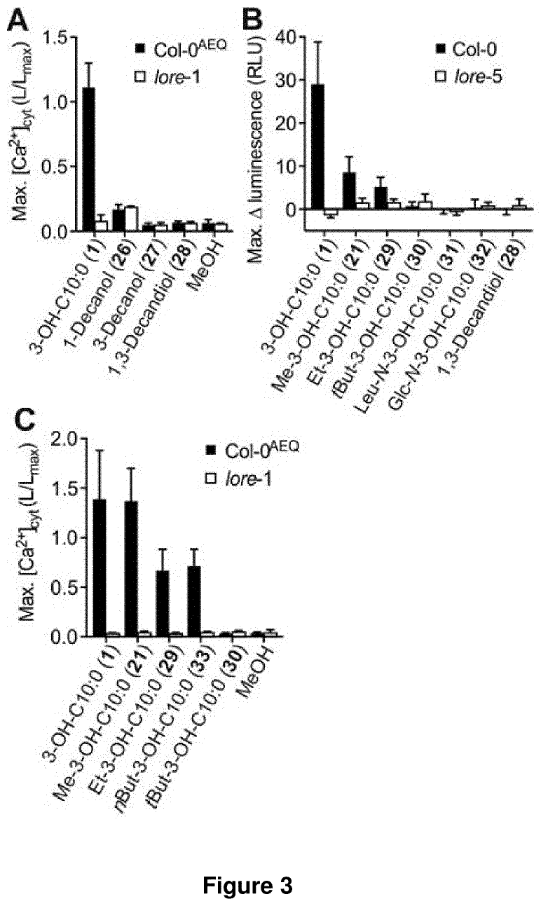

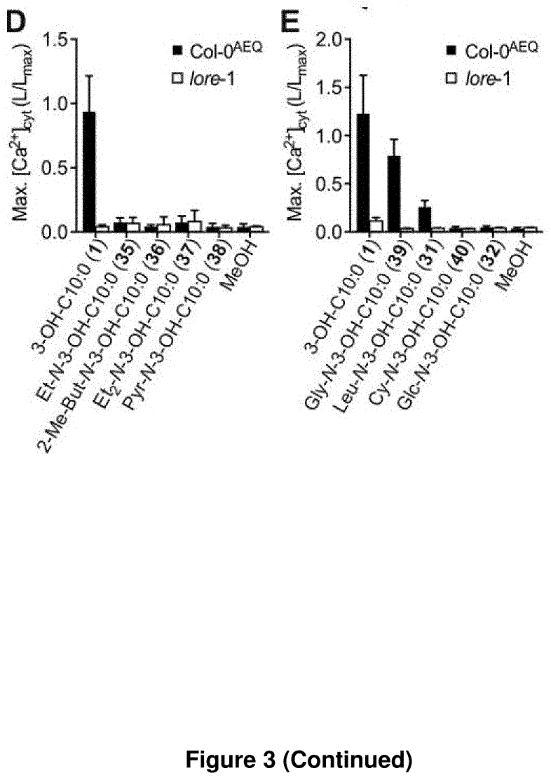

Next, it was explored if a medium chain length and a 3-hydroxyl group are sufficient to activate LORE signalling. However, neither 3-decanol nor 1,3-decandiol elicited PTI responses in Arabidopsis (FIG. 3A,B), suggesting that also the terminal carboxyl function is required for LORE-mediated immune sensing. Indeed, ester- or amide-linked moieties at the carboxyl group of 3-OH—C10:0 (21, 29-40, 46) gradually impaired elicitor activity with increasing hydrophobicity and size, with bulky moieties, such as tert-Butyl (30) or glucosamine (32), rendering the 3-OH—C10:0 derivatives inactive (FIG. 3B-E; FIG. 6B). In conclusion, mc-3-OH-FAs are the minimal motif necessary and sufficient to trigger LORE-dependent immunity with free 3-OH—C10:0 (1) exhibiting the strongest elicitor activity in Arabidopsis.

### Example 6: Analysis of the Composition of Starting LPS and LA Samples

Notably, because of the finding that bulky moieties linked to the carboxyl group abolished 3-OH—C10:0 (1) elicitor activity, the question arose whether mc-3-OH-FAs linked to LA are at all sensed by Arabidopsis or whether it is rather the free mc-3-OH-FAs that represent the elicitor-active molecules. Therefore, LPS and LA samples prepared in the inventors' laboratory, as well as LPS provided by several other laboratories and from commercial suppliers were analysed for the presence of unbound 3-OH—C10:0 (1) by means of UHPLC-MS/MS and stable isotope dilution analysis (SIDA-UHPLC-MS/MS_{MRM}) (example 1.10). Indeed, free 3-OH—C10:0 (1) was detected in all LPS and LA samples that activated LORE-dependent immune signalling (Table 1; Table 3). As this includes LPS samples isolated by different procedures involving multiple extraction and purification steps as well as LA samples that were generated through acidic hydrolysis of LPS at 100° C., subsequent solvent extraction and reversed-phase HPLC purification (Ranf et al., 2015), it was concluded that 3-OH-FAs generally co-purify with LPS/LA.

To remove unbound 3-OH-FAs from the LPS samples, ionic detergents were applied to dissociate LPS aggregates and the samples were subsequently purified either by gel permeation chromatography or repeated heat-detergent-promoted solvent extraction. Indeed, unbound mc-3-OH-FAs could only be sufficiently depleted from most LPS samples through extensive detergent treatment, as assessed by quantification of free 3-OH—C10:0 (1) (Table 1; Table 3). A part of the samples plus additional samples has been independently reassessed by quantification of free 3-OH—C10:0 (1) (Table 3).

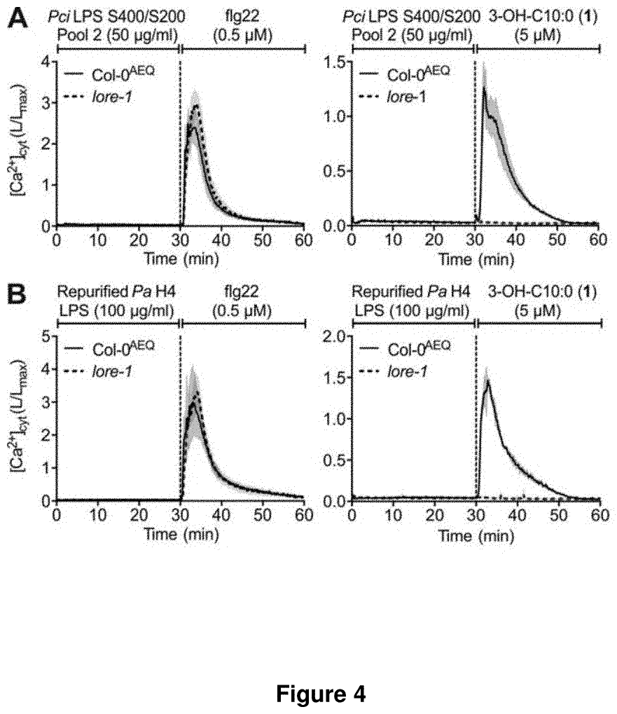

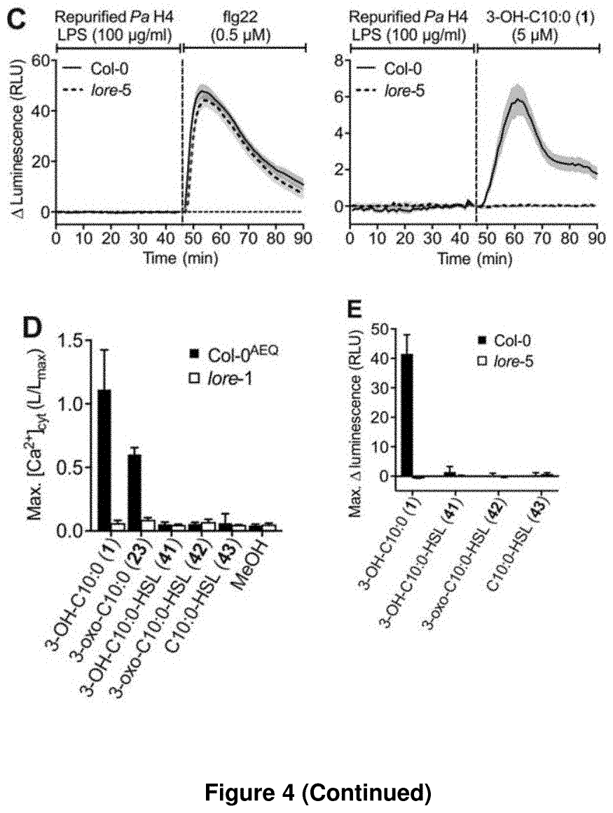

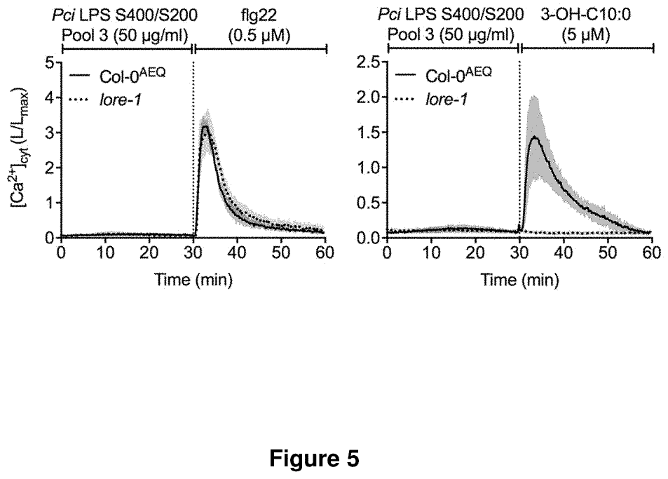

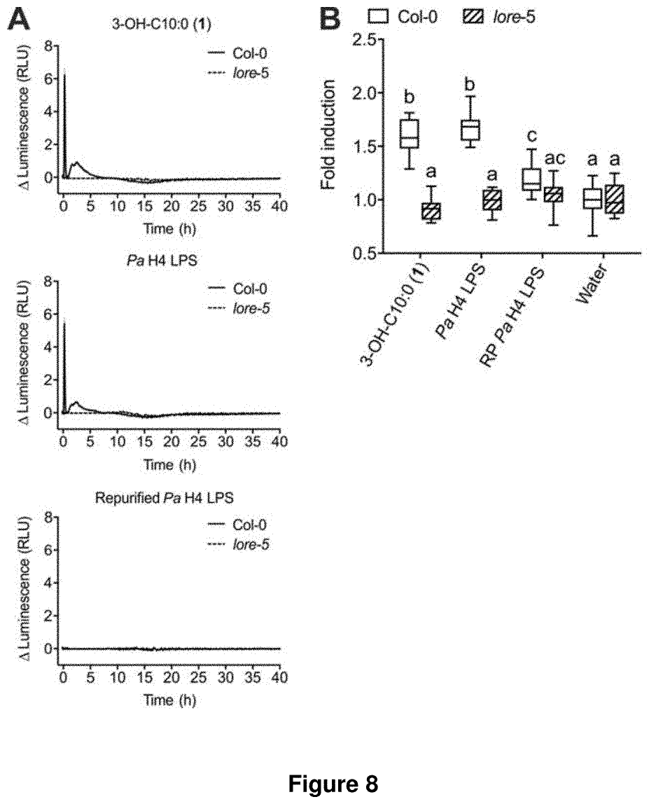

Strikingly, such mc-3-OH-FA-depleted LPS samples did not activate PTI signalling such as [Ca²⁺]_{cyt} elevations or ROS production in Arabidopsis whereas typical [Ca²⁺]_{cyt} or ROS signals were induced by subsequent application of synthetic 3-OH—C10:0 (1) or flg22, an unrelated peptide MAMP sensed by the FLAGELLIN-SENSING2 (FLS2) receptor (Felix et al., 1999) (FIG. 4A-C; FIG. 5). Mc-3-OH-FA-depleted LPS samples also did not induce late PTI responses in Arabidopsis such as a second phase of ROS production or the accumulation of peroxidases (FIG. 8A,B).

These findings suggest that the observed PTI is neither induced by intact LPS/LA, nor by mc-3-OH-FAs that are rapidly released from intact LPS/LA in the plant apoplast. Instead, these findings hint at a direct source of free, elicitor-active mc-3-OH-FAs in bacteria.

3-OH-acyl building blocks occur in several bacterial compounds, e.g. polyhydroxyalkanoates (Verlinden et al., 2007), rhamnolipids (RLs) (Abdel-Mawgoud et al., 2010a), lipopeptides (LPs) (Raaijmakers et al., 2006), and N-acyl-homoserine-lactones (acyl-HSLs) (Brelles-Mariho, 2001; Raaijmakers et al., 2010). In Pseudomonas, these are commonly mc-3-OH-acyl moieties, primarily 3-OH-decanoyl (Raaijmakers et al., 2006; Raaijmakers et al., 2010). As in these compounds either the 3-hydroxyl group and/or the carboxyl function is blocked with bulky side groups, they presumably do not activate LORE-dependent PTI directly. However, elicitor-active fragments may be released upon their degradation in planta. In support of this, synthetic 3-OH—C10:0-HSL (41) and 3-oxo-C10:0-HSL (42), for example, did not elicit significant early PTI signalling reactions upon short-term exposure in Arabidopsis (FIG. 4D-E).

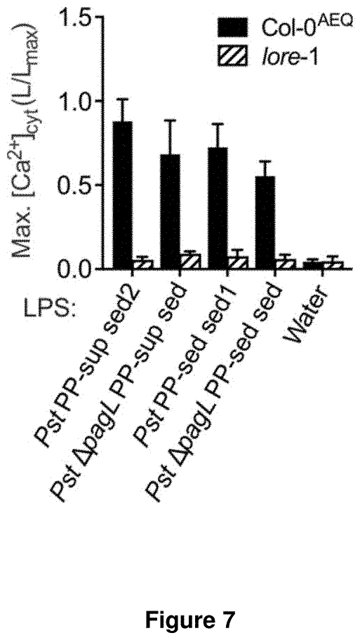

Pseudomonas releases free (R)-3-OH—C10:0 (44) during synthesis of penta-acylated lipid A through PagL-catalysed (R)-3-OH—C10:0 removal from hexa-acylated lipid A in the outer membrane (Ernst et al., 2006; Geurtsen et al., 2005). However, LPS preparations from Pseudomonas ΔpagL mutants still contain free 3-OH—C10:0 (1) and activate LORE-mediated PTI (Table 3; FIG. 7), indicating additional sources of free mc-3-OH-FAs in bacteria. Overall, these findings demonstrate that the cell-surface receptor kinase LORE mediates sensing of mc-3-OH-FAs in their free form but not as partial structures of larger entities to activate PTI in plants.

TABLE 1
List of LPS, LA, and AHL samples and quantification of free 3-OH—C10:0 (1) in these samples.

| | | | | | | | | | |
|---|---|---|---|---|---|---|---|---|---|
| | | | | | Sample conc. | | Sample conc. | | |
| | | | Further | | (used for | Free | (used for | PTI in | LORE- |
| | Bacterial | Isolation | purifi- | Source/ | quantifi- | 3-OH—C10:0 | biological | Arabi- | depen- |
| Sample | strain | procedure^{#} | cation | Reference | cation) | (1) (μM) | assays) | dopsis | dent |

Controls

| Water | — | — | — | — | — | | <LOQ^{a} | — | No | — |
|---|---|---|---|---|---|---|---|---|---|---|
| control | | | | | | | <LOQ^{b} | | | |
| 3-OH—C10:0 | — | — | — | — | 1 | μM | 0.67^{a} | 1 μM-5 μM | Yes | Yes |
| | | | | | 4 | μM | 2.49^{b} | (mostly) | | |

LPS

| | | | | | | | | | | | |
|---|---|---|---|---|---|---|---|---|---|---|---|
| Pa PAO1 | Pseudomonas | Phenol/ | — | Kooistra | 100 | μg/mL | 1.06^{$a} | 20-25 | μg/mL | Yes | Yes |
| | aeruginosa | water | | et al., | | | | | | | |
| | PAO1 | | | 2003; | | | | | | | |
| | | | | Ranf | | | | | | | |
| | | | | et al., | | | | | | | |
| | | | | 2015 | | | | | | | |
| Pa H4 | Pseudomonas | PCP | — | Sánchez | 100 | μg/mL | 0.96^{$a} | 20-25 | μg/mL | Yes | Yes |
| | aeruginosa | | | Carballo | | | | | | | |
| | H4 | | | et al., | | | | | | | |
| | | | | 1999; | | | | | | | |
| | | | | Ranf | | | | | | | |
| | | | | et al., | | | | | | | |
| | | | | 2015 | | | | | | | |
| Pst WP | Pseudomonas | Phenol/ | — | Ranf/ | 100 | μg/mL | 0.14^{$a} | 20-25 | μg/mL | Yes | Yes |
| | syringae pv. | water | | Gisch, | | | | | | | |
| | tomato | | | this | | | | | | | |
| | DC3000 | | | study | | | | | | | |
| Pst WP | Pseudomonas | Phenol/ | GPC S400 | Ranf/ | 100 | μg/mL | 2.71^{$a} | 20-25 | μg/mL | Yes | Yes |
| S400 | syringae pv. | water | | Gisch, | | | | | | | |
| | tomato | | | this | | | | | | | |
| | DC3000 | | | study | | | | | | | |
| Pst PP-sup | Pseudomonas | 1) Phenol/ | — | Ranf/ | 100 | μg/mL | 0.30^{$a} | 20-25 | μg/mL | Yes | Yes |
| sed2 | syringae pv. | water | | Gisch, | | | | | | | |
| | tomato | 2) PCP | | this | | | | | | | |
| | DC3000 | | | study | | | | | | | |
| Pst PP-sup | Pseudomonas | 1) Phenol/ | GPC S400 | Ranf/ | 100 | μg/mL | 1.64^{$a} | 20-25 | μg/mL | Yes | Yes |
| sed2 S400 | syringae pv. | water | | Gisch, | | | | | | | |
| | tomato | 2) PCP | | this | | | | | | | |
| | DC3000 | | | study | | | | | | | |
| Pst PP-sup | Pseudomonas | 1) Phenol/ | — | Ranf/ | 100 | μg/mL | 0.30^{$a} | 20-25 | μg/mL | Yes | Yes |
| sed3 | syringae pv. | water | | Gisch, | | | | | | | |
| | tomato | 2) PCP | | this | | | | | | | |
| | DC3000 | | | study | | | | | | | |
| Pst PP-sup | Pseudomonas | 1) Phenol/ | GPC S400 | Ranf/ | 100 | μg/mL | 1.95^{$a} | 20-25 | μg/mL | Yes | Yes |
| sed3 S400 | syringae pv. | water | | Gisch, | | | | | | | |
| | tomato | 2) PCP | | this | | | | | | | |
| | DC3000 | | | study | | | | | | | |
| Pst PP-sed | Pseudomonas | 1) Phenol/ | — | Ranf/ | 100 | μg/mL | 0.31^{$a} | 20-25 | μg/mL | Yes | Yes |
| sed1 | syringae pv. | water | | Gisch, | | | | | | | |
| | tomato | 2) PCP | | this | | | | | | | |
| | DC3000 | | | study | | | | | | | |
| Pst PP-sed | Pseudomonas | 1) Phenol/ | — | Ranf/ | 100 | μg/mL | 0.23^{$a} | 20-25 | μg/mL | Yes | Yes |
| sed2 | syringae pv. | water | | Gisch, | | | | | | | |
| | tomato | 2) PCP | | this | | | | | | | |
| | DC3000 | | | study | | | | | | | |
| Pci | Pseudomonas | Phenol/ | — | Ranf/ | 100 | μg/mL | 0.16^{$a} | 20-25 | μg/mL | Yes | Partially |
| | cichorii | water | | Gisch, | | | | | | | |
| | ATCC10857/ | | | this | | | | | | | |
| | DSM50259 | | | study | | | | | | | |
| Pci S400 | Pseudomonas | Phenol/ | GPC S400 | Ranf/ | 100 | μg/mL | 1.12^{$a} | 20-25 | μg/mL | Yes | Yes |
| | cichorii | water | | Gisch, | | | | | | | |
| | ATCC10857/ | | | this | | | | | | | |
| | DSM50259 | | | study | | | | | | | |
| R. sph. | Rhodobacter | — | — | InvivoGen | 100 | μg/mL | 0.64^{$a} | 20-25 | μg/mL | Yes | Yes |
| | sphaeroides | | | | | | | | | | |
| R. sph. | Rhodobacter | — | Ultrapure | InvivoGen | 100 | μg/mL | 2.15^{$a} | 20-25 | μg/mL | Yes | Yes |
| (UP) | sphaeroides | | | | | | | | | | |
| R. gel. | Rubrivivax | PCP | — | (Masoud | 1 | mg/mL | 9.97^{$b} | 20-25 | μg/mL | Yes | Yes |
| | gelatinosus | | | et al., | | | | | | | |
| | Dr2 | | | 1990) | | | | | | | |
| C. viol. | Chromo- | Phenol/ | — | Chris | 1 | mg/mL | 4.55^{$b} | 20-25 | μg/mL | Yes | Yes |
| | bacterium | water | | Galanos, | | | | | | | |
| | violaceum | | | (Hase and | | | | | | | |
| | | | | Rietschel, | | | | | | | |
| | | | | 1977) | | | | | | | |
| E. coli | Escherichia | — | Ultrapure | InvivoGen | 1 | mg/mL | <LOQ^{b} | 20-50 | μg/mL | No | — |
| O55:B5 | coli | | | | | | | | | | |
| | O55:B5 | | | | | | | | | | |
| E. coli | Escherichia | — | Ultrapure | InvivoGen | 1 | mg/mL | <LOQ^{b} | 20-50 | μg/mL | No | — |
| K12 | coli | | | | | | | | | | |
| | K12 | | | | | | | | | | |
| Pa F1 | P. aeruginosa | Phenol/ | — | (Knirel | 100 | μg/mL | 0.50^{$a} | 20-50 | μg/mL | Yes | Yes |
| | Fisher-type 1 | water | | et al., | | | | | | | |
| | (F1) (Habs 6) | | | 1985; | | | | | | | |
| | | | | Bystrova | | | | | | | |
| | | | | et al., | | | | | | | |
| | | | | 2002; | | | | | | | |
| | | | | Rant | | | | | | | |
| | | | | et al., | | | | | | | |
| | | | | 2015) | | | | | | | |
| Pa PAO1 | Pseudomonas | PCP | — | (Kooistra | 100 | μg/mL | 0.37^{$a} | 20-50 | μg/mL | Yes | Yes |
| ΔalgC | aeruginosa | | | et al., | | | | | | | |
| | PAO1 ΔalgC | | | 2003; | | | | | | | |
| | | | | Ranf | | | | | | | |
| | | | | et al., | | | | | | | |
| | | | | 2015) | | | | | | | |
| Pa R5 | Pseudomonas | PCP | — | (Masoud | 100 | μg/mL | 0.26^{$a} | 20-50 | μg/mL | Yes | Yes |
| | aeruginosa | | | et al., | | | | | | | |
| | R5 | | | 1994; | | | | | | | |
| | | | | Ranf | | | | | | | |
| | | | | et al., | | | | | | | |
| | | | | 2015) | | | | | | | |
| Pa PAN1 | Pseudomonas | PCP | — | (Bitter | 100 | μg/mL | 0.76^{$a} | 20-50 | μg/mL | Yes | Yes |
| | aeruginosa | | | et al., | | | | | | | |
| | PAN1 | | | 2007; | | | | | | | |
| | | | | Ranf | | | | | | | |
| | | | | et al., | | | | | | | |
| | | | | 2015) | | | | | | | |
| Pa PAC | Pseudomonas | PCP | — | (Kooistra | 100 | μg/mL | 0.95^{$a} | 20-50 | μg/mL | Yes | Yes |
| 1R ΔalgC | aeruginosa | | | et al., | | | | | | | |
| | PAC 1R ΔalgC | | | 2003; | | | | | | | |
| | | | | Ranf | | | | | | | |
| | | | | et al., | | | | | | | |
| | | | | 2015) | | | | | | | |
| Pst PCP | Pseudomonas | 1) Phenol/ | — | Ranf/ | 100 | μg/mL | 0.28^{$a} | 20-50 | μg/mL | Yes | Yes |
| | syringae | water | | Gisch, | | | | | | | |
| | pv. tomato | 2) PCP | | (Ranf | | | | | | | |
| | DC3000 | | | et al., | | | | | | | |
| | | | | 2015) | | | | | | | |
| Psa | Pseudomonas | Phenol/ | — | Ranf | 100 | μg/mL | 0.38^{$a} | 20-50 | μg/mL | Yes | Yes |
| | syringae | water | | et al., | | | | | | | |
| | pv. apii | | | 2015 | | | | | | | |
| Palc | Pseudomonas | Phenol/ | — | Ranf | 100 | μg/mL | 0.46^{$a} | 20-50 | μg/mL | Yes | Yes |
| | alcaligenes | water | | et al., | | | | | | | |
| | 537 | | | 2015 | | | | | | | |
| Pfluor | Pseudomonas | PCP | — | Knirel | 100 | μg/mL | 0.62^{$a} | 20-50 | μg/mL | Yes | Yes |
| | fluorescens | | | et al., | | | | | | | |
| | ATCC49271 | | | 1996; | | | | | | | |
| | | | | Ranf | | | | | | | |
| | | | | et al., | | | | | | | |
| | | | | 2015 | | | | | | | |
| Xcm (1) | Xanthomonas | Phenol/ | — | Senchenkova | 1 | mg/mL | 0.08^{$b} | 20-50 | μg/mL | Yes | Yes |
| | campestris pv. | water | | et al., | | | | | | | |
| | malvacearum | | | 2002; | | | | | | | |
| | GSPB1386 | | | Ranf | | | | | | | |
| | | | | et al., | | | | | | | |
| | | | | 2015 | | | | | | | |
| Xcm (2) | Xanthomonas | Phenol/ | — | Senchenkova | 1 | mg/mL | 0.12^{$b} | 20-50 | μg/mL | Yes | Yes |
| | campestris pv. | water | | et al., | | | | | | | |
| | malvacearum | | | 2002; | | | | | | | |
| | GSPB2388 | | | Ranf | | | | | | | |
| | | | | et al., | | | | | | | |
| | | | | 2015 | | | | | | | |
| Xcph | Xanthomonas | Phenol/ | — | Senchenkova | 1 | mg/mL | 0.06^{$b} | 20-50 | μg/mL | Yes | Yes |
| | campestris pv. | water | | et al., | | | | | | | |
| | phaseoli | | | 2002; | | | | | | | |
| | var. fuscans | | | Ranf | | | | | | | |
| | GSPB271 | | | et al., | | | | | | | |
| | | | | 2015 | | | | | | | |
| Xcb | Xanthomonas | Phenol/ | — | Senchenkova | 1 | mg/mL | 0.26^{$b} | 20-50 | μg/mL | Yes | Yes |
| | campestris pv. | water | | et al., | | | | | | | |
| | begoniae | | | 1999; | | | | | | | |
| | GSPB525 | | | Ranf | | | | | | | |
| | | | | et al., | | | | | | | |
| | | | | 2015 | | | | | | | |
| Bcp | Burkholderia | Phenol/ | — | Isshiki | 1 | mg/mL | <LOQ^{b} | 20-50 | μg/mL | No | — |
| | cepacia | water | | et al., | | | | | | | |
| | GIFU645/ | | | 1998; | | | | | | | |
| | ATCC25416 | | | Ranf | | | | | | | |
| | | | | et al., | | | | | | | |
| | | | | 2015 | | | | | | | |
| Bpm | Burkholderia | Phenol/ | — | Kawahara | 1 | mg/mL | <LOQ^{b} | 20-50 | μg/mL | No | — |
| | pseudomallei | water | | et al., | | | | | | | |
| | GIFU12046/ | | | 1992; | | | | | | | |
| | 3P-62 | | | Ranf | | | | | | | |
| | | | | et al., | | | | | | | |
| | | | | 2015 | | | | | | | |
| E. coli | Escherichia | — | Ultrapure | InvivoGen | 1 | mg/mL | <LOQ^{b} | 20-50 | μg/mL | No | — |
| O111:B4 | coli | | | | | | | | | | |
| | O111:B4 | | | | | | | | | | |
| E. coli | Escherichia | PCP | HPLC | Mamat | 1 | mg/mL | <LOQ^{b} | 20-50 | μg/mL | No | — |
| Kdo₂-LA | coli | | | et al., | | | | | | | |
| | KPM 53 | | | 2009; | | | | | | | |
| | | | | Ranf | | | | | | | |
| | | | | et al., | | | | | | | |
| | | | | 2015 | | | | | | | |
| Sm Typh | Salmonella | — | TLRgrade | Enzo Life | 1 | mg/mL | <LOQ^{b} | 20-50 | μg/mL | No | — |
| | enterica sv. | | | Sciences | | | | | | | |
| | Typhimurium | | | | | | | | | | |
| Sm | Salmonella | — | TLRgrade | Enzo Life | 1 | mg/mL | <LOQ^{b} | 20-50 | μg/mL | No | — |
| Minnesota | enterica | | | Sciences | | | | | | | |
| | Minnesota | | | | | | | | | | |
| Sm R595 | Salmonella | — | Ultrapure | InvivoGen | 1 | mg/mL | <LOQ^{b} | 20-50 | μg/mL | No | — |
| | enterica sv. | | | | | | | | | | |
| | Minnesota | | | | | | | | | | |
| | R595 | | | | | | | | | | |
| Pa H4 | P. aeruginosa | PCP | Hydrazi- | Ranf/ | 1 | mg/mL | <LOQ^{b} | 20-50 | μg/mL | No | — |
| LPS-OH | H4 | | nolysis | Gisch, | | | | | | | |
| | | | | Ranf | | | | | | | |
| | | | | et al., | | | | | | | |
| | | | | 2015 | | | | | | | |
| Pa H4 | P. aeruginosa | PCP | 1) NaAc | Ranf/ | 1 | mg/mL | <LOQ^{b} | 20-50 | μg/mL | No | — |
| core OS | H4 | | hydrolysis | Gisch, | | | | | | | |
| | | | 2) GPC of | Ranf | | | | | | | |
| | | | core oligo- | et al., | | | | | | | |
| | | | saccharides | 2015 | | | | | | | |

Lipid A

| | | | | | | | | | | | |
|---|---|---|---|---|---|---|---|---|---|---|---|
| Pa H4 | P. aeruginosa | 1) Phenol/ | 1) NaAc | Ranf/ | 100 | μg/mL | 0.47^{$a} | 25 | μg/mL | Yes | Yes |
| LA (1) | H4 | water | hydrolysis | Gisch, | | | | | | | |
| | | 2) PCP | 2) HPLC | Ranf | | | | | | | |
| | | | | et al., | | | | | | | |
| | | | | 2015 | | | | | | | |
| Pa H4 | P. aeruginosa | 1) Phenol/ | 1) NaAc | Ranf/ | 100 | μg/mL | 0.34^{$a} | 25 | μg/mL | Yes | Yes |
| LA (2) | H4 | water | hydrolysis | Gisch, | | | | | | | |
| | | 2) PCP | 2) HPLC | Ranf | | | | | | | |
| | | | | et al., | | | | | | | |
| | | | | 2015 | | | | | | | |
| Pst WP LA | Pseudomonas | Phenol/ | 1) NaAc | Ranf/ | 100 | μg/mL | 0.05^{$a} | 25 | μg/mL | Very | Yes |
| (pool 1) | syringae | water | hydrolysis | Gisch, | | | | | | weak | |
| | pv. tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.05^{$a} | 25 | μg/mL | Weak | Yes |
| (pool 2) | syringae | water | hydrolysis | Gisch, | | | | | | | |
| | pv. tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.07^{$a} | 25 | μg/mL | Weak | Yes |
| (pool 3) | syringae | water | hydrolysis | Gisch, | | | | | | | |
| | pv. tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.14^{$a} | 25 | μg/mL | Yes | Yes |
| (pool 4) | syringae | water | hydrolysis | Gisch, | | | | | | | |
| | pv. tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.23^{$a} | 25 | μg/mL | Yes | Yes |
| (pool 5) | syringae | water | hydrolysis | Gisch, | | | | | | | |
| | pv. tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.16^{$a} | 25 | μg/mL | Yes | Yes |
| (pool 6) | syringae | water | hydrolysis | Gisch, | | | | | | | |
| | pv. tomato | | 2) HPLC | this | | | | | | | |
| | DC3000 | | | study | | | | | | | |
| Pci LA | Pseudomonas | Phenol/ | 1) NaAc | Ranf/ | 100 | μg/mL | 0.35^{$a} | 25 | μg/mL | Yes | Yes |
| (pool 1) | cichorii | water | hydrolysis | Gisch, | | | | | | | |
| | ATCC10857/ | | 2) HPLC | this | | | | | | | |
| | DSM50259 | | | study | | | | | | | |
| Pci LA | Pseudomonas | Phenol/ | 1) NaAc | Ranf/ | 100 | μg/mL | 0.76^{$a} | 25 | μg/mL | Yes | Yes |
| (pool 2) | cichorii | water | hydrolysis | Gisch, | | | | | | | |
| | ATCC10857/ | | 2) HPLC | this | | | | | | | |
| | DSM50259 | | | study | | | | | | | |

Acyl-HSL

| MeOH control | — | — | — | — | — | | <LOQ | — | | No | — |
|---|---|---|---|---|---|---|---|---|---|---|---|
| C10:0-HSL | synthetic | — | — | Sigma- | 1 | mg/mL | <LOQ | 5 | μM | No | — |
| | | | | Aldrich | (3.92 | mM) | | | | | |
| 3-OH—C10:0- | synthetic | — | — | University | 1 | mg/mL | 0.80 | 5 | μM | No | — |
| HSL | | | | of | (3.69 | mM) | | | | | |
| | | | | Nottingham | | | | | | | |
| 3-oxo-C10:0- | synthetic | — | — | Sigma- | 1 | mg/mL | <LOQ | 5 | μM | No | — |
| HSL | | | | Aldrich | (3.71 | mM) | | | | | |

DOC-GPC LPS repurification

| Water control | — | — | — | — | — | | <LOQ | | | No | — |
|---|---|---|---|---|---|---|---|---|---|---|---|
| 3-OH—C10:0 | — | — | — | — | 20 | μM | 27.7 | 5 | μM | Yes | Yes |
| Pci S400/S200 | Pseudomonas | Phenol/ | GPC S400 | Ranf/ | 200 | μg/mL | <LOQ | 50 | μg/mL | No | — |
| pool 2 | cichorii | water | & DOC- | Gisch, | | | | | | | |
| | ATCC10857/ | | GPC S200 | this | | | | | | | |
| | DSM50259 | | | study | | | | | | | |
| Pci S400/S200 | Pseudomonas | Phenol/ | GPC S400 | Ranf/ | 200 | μg/mL | 0.72 | 50 | μg/mL | Very | Yes |
| pool 3 | cichorii | water | & DOC- | Gisch, | | | | | | weak | |
| | ATCC10857/ | | GPC S200 | this | | | | | | | |
| | DSM50259 | | | study | | | | | | | |
| Pci S400/S200 | Pseudomonas | Phenol/ | GPC S400 | Ranf/ | 200 | μg/mL | <LOQ | 50 | μg/mL | No | — |
| pool 4 | cichorii | water | & DOC- | Gisch, | | | | | | | |
| | ATCC10857/ | | GPC S200 | this | | | | | | | |
| | DSM50259 | | | study | | | | | | | |

Heat-detergent-promoted LPS repurification

| Water control | — | — | — | — | — | | <LOQ | | | No | — |
|---|---|---|---|---|---|---|---|---|---|---|---|
| 3-OH—C10:0 | — | — | — | — | 20 | μM | 23.4 | 5 | μM | Yes | Yes |
| Pa H4 | Pseudomonas | PCP | — | Sánchez | 100 | μg/mL | 4.67^{$} | 25 | μg/mL | Yes | Yes |
| | aeruginosa | | | Carballo | | | | | | | |
| | H4 | | | et al., | | | | | | | |
| | | | | 1999; | | | | | | | |
| | | | | Ranf | | | | | | | |
| | | | | et al., | | | | | | | |
| | | | | 2015 | | | | | | | |
| Repurified | Pseudomonas | PCP | Heat- | Ranf/ | 400 | μg/mL | <LOQ | 100 | μg/mL | No | — |
| Pa H4 | aeruginosa | | detergent | Gisch, | | | | | | | |
| | H4 | | repurifi- | this | | | | | | | |
| | | | cation | study | | | | | | | |

^{#}LPS preparations were generated using phenol/water extraction (Westphal and Jann, 1965), phenol-chloroform-petroleum ether (PCP) extraction (Galanos et al., 1969) or a combination of the two protocols in the indicated order.
^{$}Note that the total content of 3-OH—C10:0 in LPS samples is presumably significantly higher because only free 3-OH—C10:0 molecules (dissolved from LPS aggregates/micelles) but not the fraction still aggregated with LPS is considered under the assay conditions.
^{a, b}Letter labels indicate the respective controls belonging to this set of samples.

TABLE 2
List of synthetic compounds 1-46 tested in this study.

| Compound | | | |
|---|---|---|---|
| acronym | | | Source/ |
| and (no.) | IUPAC name | Structural formula | Reference |
| 3-OH-C10:0 (1) | 3-hydroxydecanoic acid | 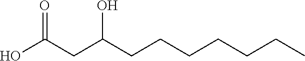 | Matreya LLC, M-1727 & synthesized in this study |
| 3-OH-C8:0 (2) | 3-hydroxyoctanoic acid | 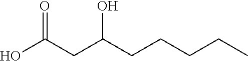 | Matreya LLC, M-1745 |
| 3-OH-C9:0 (3) | 3-hydroxynonanoic acid | 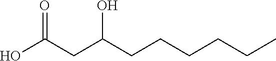 | Matreya LLC, M-1725 |
| 3-OH-C11:0 (4) | 3-hydroxyundecanoic acid | 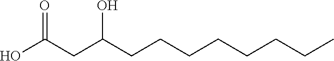 | Matreya LLC, M-1729 |
| 3-OH-C12:0 (5) | 3-hydroxydodecanoic acid | 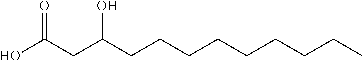 | Matreya LLC, M-1731 |
| 3-OH-C13:0 (6) | 3-hydroxytridecanoic acid | 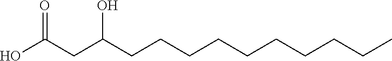 [SMILES: CCCCCCCCCCC(O)CC(=O)O] | Matreya LLC, M-1733 |
| 3-OH-C14:0 (7) | 3-hydroxytetradecanoic acid | 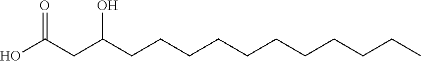 [SMILES: CCCCCCCCCCCC(O)CC(=O)O] | Matreya LLC, M-1735 |
| 3-OH-C16:0 (8) | 3-hydroxydhexadecanoic acid | 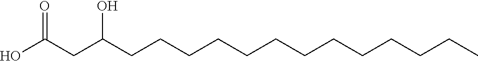 [SMILES: CCCCCCCCCCCCCC(O)CC(=O)O] | Matreya LLC, M-1739 |
| 2-OH-C8:0 (9) | 2-hydroxyoctanoic acid | 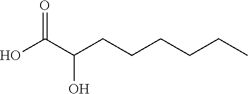 | Sigma- Aldrich, Taufkirchen, Germany, H7396 |
| 2-OH-C10:0 (10) | 2-hydroxydecanoic acid | 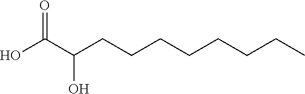 | Matreya LLC, M-1758 |
| 2-OH-C12:0 (11) | 2-hydroxydodecanoic acid | 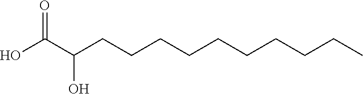 | Matreya LLC, M-1701 |
| 2-OH-C14:0 (12) | 2-hydroxytetradecanoic acid | 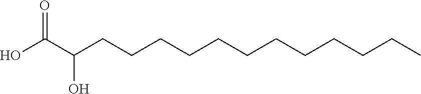 [SMILES: CCCCCCCCCCCCC(O)C(=O)O] | Matreya LLC, M-1703 |
| C8:0 (13) | octanoic acid (caprylic acid) | 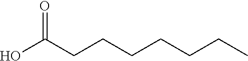 | Sigma- Aldrich, Taufkirchen, Germany |
| | | | W279900 |
| C10:0 (14) | decanoic acid (capric acid) | 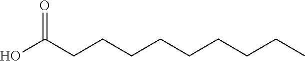 | Sigma- Aldrich, Taufkirchen, Germany |
| | | | 21409 |
| C12:0 (15) | dodecanoic acid (lauric acid) | 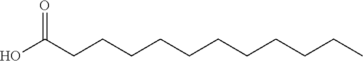 | Sigma- Aldrich, Taufkirchen, Germany, |
| | | | L4250 |
| C14:0 (16) | tetradecanoic acid (myristic acid) | 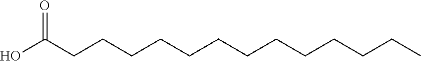 [SMILES: CCCCCCCCCCCCCC(=O)O] | Sigma- Aldrich, Taufkirchen, Germany, |
| | | | M3128 |
| cis-Δ²-C10:1 (17) | (Z)-dec-2-enoic acid | 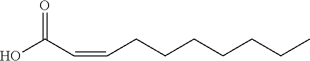 | Sigma- Aldrich, Taufkirchen, Germany, |
| | | | 19699 |
| trans-Δ²- C10:1 (18) | (E)-dec-2-enoic acid | 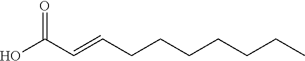 | synthesized in this study |
| 4-OH-C10:0 (19) | 4-hydroxydecanoic acid | 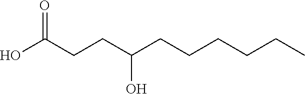 | Sigma- Aldrich, Taufkirchen, Germany, D804 |
| 5-OH-C10:0 (20) | 5-hydroxydecanoic acid | 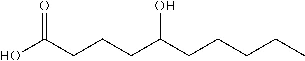 | Sigma- Aldrich, Taufkirchen, Germany, |
| | | | H135 |
| Me-3-OH- C10:0 (21) | methyl 3- hydroxydecanoate | 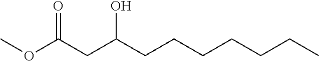 | Matreya LLC, M-1728 |
| Me-3-Cl- C10:0 (22) | methyl 3-chlorodecanoate | 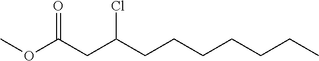 | synthesized in this study |
| 3-oxo-C10:0 (23) | 3-oxodecanoic acid | 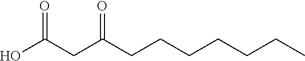 | synthesized in this study |
| 3-MeO- C10:0 (24) | 3-methoxydecanoic acid | 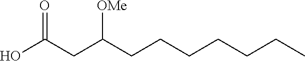 | synthesized in this study |
| 3-AcO- C10:0 (25) | 3-acetoxydecanoic acid | 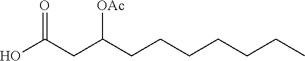 [SMILES: CCCCCCCC(CC(=O)O)OC(C)=O] | synthesized in this study |
| 1-Decanol (26) | decan-1-ol | 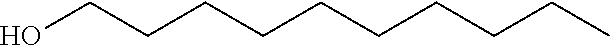 | Sigma- Aldrich, |
| | | | Taufkirchen, |
| | | | Germany, |
| | | | 239763 |
| 3-Decanol (27) | decan-3-ol | 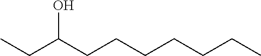 | Sigma- Aldrich, Taufkirchen, Germany, |
| | | | CDS001495 |
| 1,3- Decandiol (28) | decane-1,3-diol | 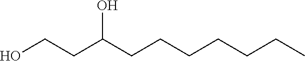 | synthesized in this study |
| Et-3-OH- C10:0 (29) | ethyl 3-hydroxydecanoate | 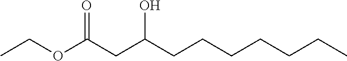 | synthesized in this study |
| tBut-3-OH- C10:0 (30) | tert-butyl-3- hydroxydecanoate | 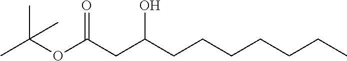 [SMILES: CCCCCCCC(O)CC(=O)OC(C)(C)C] | synthesized in this study |
| Leu-N-3- OH-C10:0 (31) | (3-hydroxydecanoyl)-L- leucine | 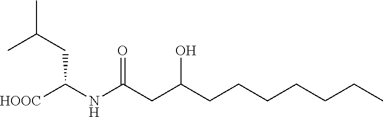 [SMILES: CCCCCCCC(O)CC(=O)N[C@@H](CC(C)C)C(=O)O] | synthesized in this study |
| Glc-N-3-OH- C10:0 (32) | 3-hydroxy-N- ((2S,3R,4R,5S,6R)-2,4,5- trihydroxy-6- (hydroxymethyl)tetrahydro- 2H-pyran-3-yl)decanamide | 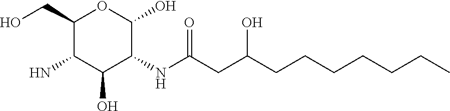 [SMILES: CCCCCCCC(O)CC(=O)N[C@@H]1[C@@H](O)[C@H](N)[C@@H](CO)O[C@@H]1O] | synthesized in this study |
| nBut-3-OH- C10:0 (33) | butyl 3-hyroxydecanoate | 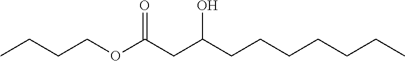 [SMILES: CCCCCCCC(O)CC(=O)OCCCC] | synthesized in this study |
| Me-3-OH- C12:0 (34) | methyl 3- hydroxydodecanoate | 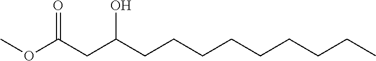 [SMILES: CCCCCCCCCC(O)CC(=O)OC] | Matreya LLC, M-1732 |
| Et-N-3-OH- C10:0 (35) | N-ethyl-3- hydroxydecanamide | 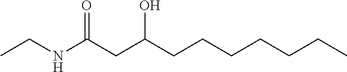 | synthesized in this study |
| 2-Me-But-N- 3-OH-C10:0 (36) | 3-hydroxy-N-((S)-2- methylbutyl)decanamide | 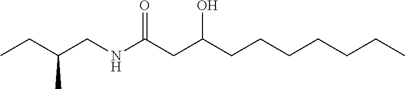 [SMILES: CCCCCCCC(O)CC(=O)NC[C@@H](C)CC] | synthesized in this study |
| Et₂-N-3-OH- C10:0 (37) | N,N-diethyl-3- hydroxydecanamide | 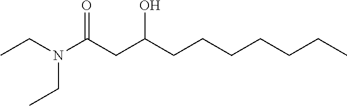 [SMILES: CCCCCCCC(O)CC(=O)N(CC)CC] | synthesized in this study |
| Pyr-N-3-OH- C10:0 (38) | 3-hydroxy-1-(pyrrolidin-1- yl)decan-1-one | 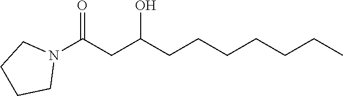 [SMILES: CCCCCCCC(O)CC(=O)N1CCCC1] | synthesized in this study |
| Gly-N-3-OH- C10:0 (39) | (3- hydroxydecanoyl)glycine | 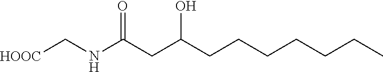 [SMILES: CCCCCCCC(O)CC(=O)NCC(=O)O] | synthesized in this study |
| Cy-N-3-OH- C10:0 (40) | N-cyclohexyl-3- hydroxydecanamide | 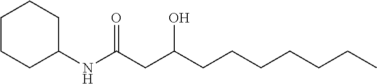 [SMILES: CCCCCCCC(O)CC(=O)NC1CCCCC1] | synthesized in this study |
| 3-OH-C10:0- HSL (41) | N-(3-Hydroxydecanoyl)-L- homoserine lactone | 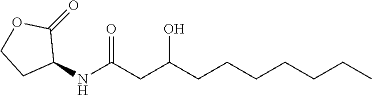 [SMILES: CCCCCCCC(O)CC(=O)N[C@H]1CCOC1=O] | Univ. of Nottingham HC10 |
| 3-oxo- C10:0-HSL (42) | N-(3-Oxodecanoyl)-L- homoserine lactone | 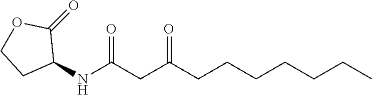 [SMILES: CCCCCCCC(=O)CC(=O)N[C@H]1CCOC1=O] | Sigma- Aldrich, Taufkirchen, Germany, O9014 |
| C10:0-HSL (43) | N-Decanoyl-DL- homoserine lactone | 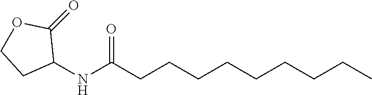 [SMILES: CCCCCCCCCC(=O)NC1CCOC1=O] | Sigma- Aldrich, Taufkirchen, Germany, 17248 |
| (R)-3-OH- C10:0 (44) | (R)-3-hydroxydecanoic acid | 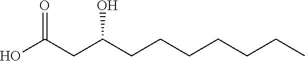 | synthesized in this study from Me-(R)- 3-OH-C10:0 |
| | | | Carbosynth, |
| | | | Compton |
| | | | Berkshire, |
| | | | United |
| | | | Kingdom, |
| | | | FH24326 |
| (S)-3-OH- C10:0 (45) | (S)-3-hydroxydecanoic acid | 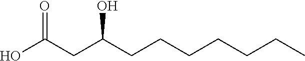 | Chemspace, Riga, Lativa, FCH1217832 |
| Me-Glly-N-3- OH-C10:0 (46) | methyl (3- hydroxydecanoyl)glycinate | 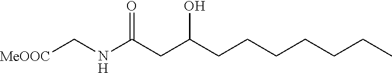 [SMILES: CCCCCCCC(O)CC(=O)NCC(=O)OC] | synthesized in this study |

TABLE 3
List of LPS, LA, and AHL samples and quantification of free 3-OH—C10:0 (1) in these samples.

| | | | | | | | | | |
|---|---|---|---|---|---|---|---|---|---|
| | | | | | Sample conc. | | Sample conc. | | |
| | | | Further | | (used for | Free | (used for | PTI in | LORE- |
| | Bacterial | Isolation | purifi- | Source/ | quantifi- | 3-OH—C10:0 | biological | Arabi- | depen- |
| Sample | strain | procedure^{#} | cation | Reference | cation) | (1) (μM) | assays) | dopsis | dent |

Controls

| Water control | — | — | — | — | — | | <LOQ | — | No | — |
|---|---|---|---|---|---|---|---|---|---|---|
| 3-OH—C10:0 | — | — | — | — | 4 | μM | 2.49 | 1 μM-5 μM | Yes | Yes |
| | | | | | | | | (mostly) | | |

LPS

| | | | | | | | | | | | |
|---|---|---|---|---|---|---|---|---|---|---|---|
| Pa PAO1 | Pseudomonas | Phenol/ | — | Kooistra | 100 | μg/mL | 0.50^{$} | 20-25 | μg/mL | Yes | Yes |
| | aeruginosa | water | | et al., | | | | | | | |
| | PAO1 | | | 2003; | | | | | | | |
| | | | | Ranf | | | | | | | |
| | | | | et al., | | | | | | | |
| | | | | 2015 | | | | | | | |
| Pa H4 | Pseudomonas | PCP | — | Sánchez | 100 | μg/mL | 0.95^{$} | 20-25 | μg/mL | Yes | Yes |
| | aeruginosa | | | Carballo | | | | | | | |
| | H4 | | | et al., | | | | | | | |
| | | | | 1999; | | | | | | | |
| | | | | Ranf | | | | | | | |
| | | | | et al., | | | | | | | |
| | | | | 2015 | | | | | | | |
| Pst WP | Pseudomonas | Phenol/ | — | Ranf/ | 100 | μg/mL | 0.14^{$} | 20-25 | μg/mL | Yes | Yes |
| | syringae pv. | water | | Gisch, | | | | | | | |
| | tomato | | | this | | | | | | | |
| | DC3000 | | | study | | | | | | | |
| Pst WP | Pseudomonas | Phenol/ | GPC S400 | Ranf/ | 100 | μg/mL | 2.71^{$} | 20-25 | μg/mL | Yes | Yes |
| S400 | syringae pv. | water | | Gisch, | | | | | | | |
| | tomato | | | this | | | | | | | |
| | DC3000 | | | study | | | | | | | |
| Pst PP-sup | Pseudomonas | 1) Phenol/ | — | Ranf/ | 100 | μg/mL | 0.30^{$} | 20-25 | μg/mL | Yes | Yes |
| sed2 | syringae pv. | water | | Gisch, | | | | | | | |
| | tomato | 2) PCP | | this | | | | | | | |
| | DC3000 | | | study | | | | | | | |
| Pst PP-sup | Pseudomonas | 1) Phenol/ | GPC S400 | Ranf/ | 100 | μg/mL | 1.64^{$} | 20-25 | μg/mL | Yes | Yes |
| sed2 S400 | syringae pv. | water | | Gisch, | | | | | | | |
| | tomato | 2) PCP | | this | | | | | | | |
| | DC3000 | | | study | | | | | | | |
| Pst PP-sup | Pseudomonas | 1) Phenol/ | — | Ranf/ | 100 | μg/mL | 0.30^{$} | 20-25 | μg/mL | Yes | Yes |
| sed3 | syringae pv. | water | | Gisch, | | | | | | | |
| | tomato | 2) PCP | | this | | | | | | | |
| | DC3000 | | | study | | | | | | | |
| Pst PP-sup | Pseudomonas | 1) Phenol/ | GPC S400 | Ranf/ | 100 | μg/mL | 1.95^{$} | 20-25 | μg/mL | Yes | Yes |
| sed3 S400 | syringae pv. | water | | Gisch, | | | | | | | |
| | tomato | 2) PCP | | this | | | | | | | |
| | DC3000 | | | study | | | | | | | |
| Pst PP-sed | Pseudomonas | 1) Phenol/ | — | Ranf/ | 100 | μg/mL | 0.31^{$} | 20-25 | μg/mL | Yes | Yes |
| sed1 | syringae pv. | water | | Gisch, | | | | | | | |
| | tomato | 2) PCP | | this | | | | | | | |
| | DC3000 | | | study | | | | | | | |
| Pst PP-sed | Pseudomonas | 1) Phenol/ | — | Ranf/ | 100 | μg/mL | 0.23^{$} | 20-25 | μg/mL | Yes | Yes |
| sed2 | syringae pv. | water | | Gisch, | | | | | | | |
| | tomato | 2) PCP | | this | | | | | | | |
| | DC3000 | | | study | | | | | | | |
| Pci | Pseudomonas | Phenol/ | — | Ranf/ | 100 | μg/mL | 0.16^{$} | 20-25 | μg/mL | Yes | Partially |
| | cichorii | water | | Gisch, | | | | | | | |
| | ATCC10857/ | | | this | | | | | | | |
| | DSM50259 | | | study | | | | | | | |
| Pci S400 | Pseudomonas | Phenol/ | GPC S400 | Ranf/ | 100 | μg/mL | 1.12^{$} | 20-25 | μg/mL | Yes | Yes |
| | cichorii | water | | Gisch, | | | | | | | |
| | ATCC10857/ | | | this | | | | | | | |
| | DSM50259 | | | study | | | | | | | |
| R. sph. | Rhodobacter | — | — | InvivoGen | 100 | μg/mL | 1.97^{$} | 20-25 | μg/mL | Yes | Yes |
| | sphaeroides | | | | | | | | | | |
| R. sph. | Rhodobacter | — | Ultrapure | InvivoGen | 100 | μg/mL | 0.62^{$} | 20-25 | μg/mL | Yes | Yes |
| (UP) | sphaeroides | | | | | | | | | | |
| R. gel. | Rubrivivax | PCP | — | (Masoud | 100 | μg/mL | 2.15^{$} | 20-25 | μg/mL | Yes | Yes |
| | gelatinosus | | | et al., | | | | | | | |
| | Dr2 | | | 1990) | | | | | | | |
| C. viol. | Chromo- | Phenol/ | — | Chris | 100 | μg/mL | 0.64^{$} | 20-25 | μg/mL | Yes | Yes |
| | bacterium | water | | Galanos, | | | | | | | |
| | violaceum | | | (Hase and | | | | | | | |
| | | | | Rietschel, | | | | | | | |
| | | | | 1977) | | | | | | | |
| E. coli | Escherichia | — | Ultrapure | InvivoGen | 1 | mg/mL | <LOQ | 20-50 | μg/mL | No | — |
| O55:B5 | coli | | | | | | | | | | |
| | O55:B5 | | | | | | | | | | |
| E. coli K12 | Escherichia | — | Ultrapure | InvivoGen | 1 | mg/mL | <LOQ | 20-50 | μg/mL | No | — |
| | coli | | | | | | | | | | |
| | K12 | | | | | | | | | | |
| Pa F1 | P. aeruginosa | Phenol/ | — | (Knirel | 100 | μg/mL | 0.57^{$} | 20-50 | μg/mL | Yes | Yes |
| | Fisher-type 1 | water | | et al., | | | | | | | |
| | (F1) (Habs 6) | | | 1985; | | | | | | | |
| | | | | Bystrova | | | | | | | |
| | | | | et al., | | | | | | | |
| | | | | 2002; | | | | | | | |
| | | | | Ranf | | | | | | | |
| | | | | et al., | | | | | | | |
| | | | | 2015) | | | | | | | |
| Pa PAO1 | Pseudomonas | PCP | — | (Kooistra | 100 | μg/mL | 0.28^{$} | 20-50 | μg/mL | Yes | Yes |
| ΔalgC | aeruginosa | | | et al., | | | | | | | |
| | PAO1 ΔalgC | | | 2003; | | | | | | | |
| | | | | Ranf | | | | | | | |
| | | | | et al., | | | | | | | |
| | | | | 2015) | | | | | | | |
| Pa R5 | Pseudomonas | PCP | — | (Masoud | 100 | μg/mL | 0.76^{$} | 20-50 | μg/mL | Yes | Yes |
| | aeruginosa | | | et al., | | | | | | | |
| | R5 | | | 1994; | | | | | | | |
| | | | | Ranf | | | | | | | |
| | | | | et al., | | | | | | | |
| | | | | 2015) | | | | | | | |
| Pa PAN1 | Pseudomonas | PCP | — | (Bitter | 100 | μg/mL | 0.37^{$} | 20-50 | μg/mL | Yes | Yes |
| | aeruginosa | | | et al., | | | | | | | |
| | PAN1 | | | 2007; | | | | | | | |
| | | | | Ranf | | | | | | | |
| | | | | et al., | | | | | | | |
| | | | | 2015) | | | | | | | |
| Pa PAC | Pseudomonas | PCP | — | (Kooistra | 100 | μg/mL | 1.06^{$} | 20-50 | μg/mL | Yes | Yes |
| 1R ΔalgC | aeruginosa | | | et al., | | | | | | | |
| | PAC 1R ΔalgC | | | 2003; | | | | | | | |
| | | | | Ranf | | | | | | | |
| | | | | et al., | | | | | | | |
| | | | | 2015) | | | | | | | |
| Pst PCP | Pseudomonas | 1) Phenol/ | — | Ranf/ | 100 | μg/mL | 0.38^{$} | 20-50 | μg/mL | Yes | Yes |
| | syringae pv. | water | | Gisch, | | | | | | | |
| | tomato | 2) PCP | | (Ranf | | | | | | | |
| | DC3000 | | | et al., | | | | | | | |
| | | | | 2015) | | | | | | | |
| Psa | Pseudomonas | Phenol/ | — | Ranf | 100 | μg/mL | 0.43^{$} | 20-50 | μg/mL | Yes | Yes |
| | syringae pv. | water | | et al., | | | | | | | |
| | apii | | | 2015 | | | | | | | |
| Palc | Pseudomonas | Phenol/ | — | Ranf | 100 | μg/mL | 0.26^{$} | 20-50 | μg/mL | Yes | Yes |
| | alcaligenes | water | | et al., | | | | | | | |
| | 537 | | | 2015 | | | | | | | |
| Pfluor | Pseudomonas | PCP | — | Knirel | 100 | μg/mL | 0.28^{$} | 20-50 | μg/mL | Yes | Yes |
| | fluorescens | | | et al., | | | | | | | |
| | ATCC49271 | | | 1996; | | | | | | | |
| | | | | Ranf | | | | | | | |
| | | | | et al., | | | | | | | |
| | | | | 2015 | | | | | | | |
| Xcm (1) | Xanthomonas | Phenol/ | — | Senchenkova | 100 | μg/mL | 0.09^{$} | 20-50 | μg/mL | Yes | Yes |
| | campestris pv. | water | | et al., | | | | | | | |
| | malvacearum | | | 2002; | | | | | | | |
| | GSPB1386 | | | Ranf | | | | | | | |
| | | | | et al., | | | | | | | |
| | | | | 2015 | | | | | | | |
| Xcm (2) | Xanthomonas | Phenol/ | — | Senchenkova | 100 | μg/mL | 0.11^{$} | 20-50 | μg/mL | Yes | Yes |
| | campestris pv. | water | | et al., | | | | | | | |
| | malvacearum | | | 2002; | | | | | | | |
| | GSPB2388 | | | Ranf | | | | | | | |
| | | | | et al., | | | | | | | |
| | | | | 2015 | | | | | | | |
| Xcph | Xanthomonas | Phenol/ | — | Senchenkova | 100 | μg/mL | 0.03^{$} | 20-50 | μg/mL | Yes | Yes |
| | campestris pv. | water | | et al., | | | | | | | |
| | phaseoli var. | | | 2002; | | | | | | | |
| | fuscans | | | Ranf | | | | | | | |
| | GSPB271 | | | et al., | | | | | | | |
| | | | | 2015 | | | | | | | |
| Xcb | Xanthomonas | Phenol/ | — | Senchenkova | 100 | μg/mL | 0.17^{$} | 20-50 | μg/mL | Yes | Yes |
| | campestris pv. | water | | et al., | | | | | | | |
| | begoniae | | | 1999; | | | | | | | |
| | GSPB525 | | | Ranf | | | | | | | |
| | | | | et al., | | | | | | | |
| | | | | 2015 | | | | | | | |
| Bcp | Burkholderia | Phenol/ | — | Isshiki | 1 | mg/mL | <LOQ | 20-50 | μg/mL | No | — |
| | cepacia | water | | et al., | | | | | | | |
| | GIFU645/ | | | 1998; | | | | | | | |
| | ATCC25416 | | | Ranf | | | | | | | |
| | | | | et al., | | | | | | | |
| | | | | 2015 | | | | | | | |
| Bpm | Burkholderia | Phenol/ | — | Kawahara | 1 | mg/mL | <LOQ | 20-50 | μg/mL | No | — |
| | pseudomallei | water | | et al., | | | | | | | |
| | GIFU12046/ | | | 1992; | | | | | | | |
| | 3P-62 | | | Ranf | | | | | | | |
| | | | | et al., | | | | | | | |
| | | | | 2015 | | | | | | | |
| E. coli | Escherichia | — | Ultrapure | InvivoGen | 1 | mg/mL | <LOQ | 20-50 | μg/mL | No | — |
| O111:B4 | coli | | | | | | | | | | |
| | O111:B4 | | | | | | | | | | |
| E. coli | Escherichia | PCP | HPLC | Mamat | 1 | mg/mL | <LOQ | 20-50 | μg/mL | No | — |
| Kdo₂-LA | coli | | | et al., | | | | | | | |
| | KPM 53 | | | 2009; | | | | | | | |
| | | | | Ranf | | | | | | | |
| | | | | et al., | | | | | | | |
| | | | | 2015 | | | | | | | |
| Sm Typh | Salmonella | — | TLRgrade | Enzo Life | 1 | mg/mL | <LOQ | 20-50 | μg/mL | No | — |
| | enterica sv. | | | Sciences | | | | | | | |
| | Typhimurium | | | | | | | | | | |
| Sm | Salmonella | — | TLRgrade | Enzo Life | 1 | mg/mL | <LOQ | 20-50 | μg/mL | No | — |
| Minnesota | enterica | | | Sciences | | | | | | | |
| | Minnesota | | | | | | | | | | |
| Sm R595 | Salmonella sv. | — | Ultrapure | InvivoGen | 1 | mg/mL | <LOQ | 20-50 | μg/mL | No | — |
| | enterica | | | | | | | | | | |
| | Minnesota | | | | | | | | | | |
| | R595 | | | | | | | | | | |
| Pa H4 | P. aeruginosa | PCP | Hydrazi- | Ranf/ | 1 | mg/mL | <LOQ | 20-50 | μg/mL | No | — |
| LPS-OH | H4 | | nolysis | Gisch, | | | | | | | |
| | | | | Ranf | | | | | | | |
| | | | | et al., | | | | | | | |
| | | | | 2015 | | | | | | | |
| Pa H4 | P. aeruginosa | PCP | 1) NaAc | Ranf/ | 1 | mg/mL | <LOQ | 20-50 | μg/mL | No | — |
| core OS | H4 | | hydrolysis | Gisch, | | | | | | | |
| | | | 2) GPC of | Ranf | | | | | | | |
| | | | core oligo- | et al., | | | | | | | |
| | | | saccharides | 2015 | | | | | | | |

Lipid A

| | | | | | | | | | | | |
|---|---|---|---|---|---|---|---|---|---|---|---|
| Pa H4 | P. aeruginosa | 1) Phenol/ | 1) NaAc | Ranf/ | 100 | μg/mL | 0.34^{$} | 25 | μg/mL | Yes | Yes |
| LA (1) | H4 | water | hydrolysis | Gisch, | | | | | | | |
| | | 2) PCP | 2) HPLC | Ranf | | | | | | | |
| | | | | et al., | | | | | | | |
| | | | | 2015 | | | | | | | |
| Pa H4 | P. aeruginosa | 1) Phenol/ | 1) NaAc | Ranf/ | 100 | μg/mL | 0.13^{$} | 25 | μg/mL | Yes | Yes |
| LA (2) | H4 | water | hydrolysis | Gisch, | | | | | | | |
| | | 2) PCP | 2) HPLC | Ranf | | | | | | | |
| | | | | et al., | | | | | | | |
| | | | | 2015 | | | | | | | |
| Pst WP LA | Pseudomonas | Phenol/ | 1) NaAc | Ranf/ | 100 | μg/mL | 0.05^{$} | 25 | μg/mL | Very | Yes |
| (pool 1) | syringae pv. | water | hydrolysis | Gisch, | | | | | | weak | |
| | tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.05^{$} | 25 | μg/mL | Weak | Yes |
| (pool 2) | syringae pv. | water | hydrolysis | Gisch, | | | | | | | |
| | tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.07^{$} | 25 | μg/mL | Weak | Yes |
| (pool 3) | syringae pv. | water | hydrolysis | Gisch, | | | | | | | |
| | tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.14^{$} | 25 | μg/mL | Yes | Yes |
| (pool 4) | syringae pv. | water | hydrolysis | Gisch, | | | | | | | |
| | tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.23^{$} | 25 | μg/mL | Yes | Yes |
| (pool 5) | syringae pv. | water | hydrolysis | Gisch, | | | | | | | |
| | tomato | | 2) HPLC | this | | | | | | | |
| | DC3000 | | | study | | | | | | | |
| Pst WP LA | Pseudomonas | Phenol/ | 1) NaAc | Ranf/ | 100 | μg/mL | 0.16^{$} | 25 | μg/mL | Yes | Yes |
| (pool 6) | syringae pv. | water | hydrolysis | Gisch, | | | | | | | |
| | tomato | | 2) HPLC | this | | | | | | | |
| | DC3000 | | | study | | | | | | | |
| Pci LA | Pseudomonas | Phenol/ | 1) NaAc | Ranf/ | 100 | μg/mL | 0.35^{$} | 25 | μg/mL | Yes | Yes |
| (pool 1) | cichorii | water | hydrolysis | Gisch, | | | | | | | |
| | ATCC10857/ | | 2) HPLC | this | | | | | | | |
| | DSM50259 | | | study | | | | | | | |
| Pci LA | Pseudomonas | Phenol/ | 1) NaAc | Ranf/ | 100 | μg/mL | 0.76^{$} | 25 | μg/mL | Yes | Yes |
| (pool 2) | cichorii | water | hydrolysis | Gisch, | | | | | | | |
| | ATCC10857/ | | 2) HPLC | this | | | | | | | |
| | DSM50259 | | | study | | | | | | | |

Acyl-HSL

| MeOH | — | — | — | — | | | <LOQ | — | | No | — |
|---|---|---|---|---|---|---|---|---|---|---|---|
| control | | | | | | | | | | | |
| C10:0-HSL | synthetic | — | — | Sigma- | 1 | mg/mL | <LOQ | 5 | μM | No | — |
| | | | | Aldrich | (3.92 | mM) | | | | | |
| 3-OH—C10:0- | synthetic | — | — | University | 1 | mg/mL | 0.80 | 5 | μM | No | — |
| HSL | | | | of | (3.69 | mM) | | | | | |
| | | | | Nottingham | | | | | | | |
| 3-oxo-C10:0- | synthetic | — | — | Sigma- | 1 | mg/mL | <LOQ | 5 | μM | No | — |
| HSL | | | | Aldrich | (3.71 | mM) | | | | | |

DOC-GPC LPS repurification

| Water control | — | — | — | — | — | | <LOQ | | | No | — |
|---|---|---|---|---|---|---|---|---|---|---|---|
| 3-OH—C10:0 | — | — | — | — | 20 | μM | 24.89 | 5 | μM | Yes | Yes |
| Pci S400/S200 | Pseudomonas | Phenol/ | GPC S400 & | Ranf/ | 200 | μg/mL | 0.07 | 50 | μg/mL | No | |
| pool 2 | cichorii | water | DOC-GPC | Gisch, | | | | | | | |
| | ATCC10857/ | | S200 | this | | | | | | | |
| | DSM50259 | | | study | | | | | | | |
| Pci S400/S200 | Pseudomonas | Phenol/ | GPC S400 & | Ranf/ | 200 | μg/mL | <LOQ | 50 | μg/mL | Very | Yes |
| pool 3 | cichorii | water | DOC-GPC | Gisch, | | | | | | weak | |
| | ATCC10857/ | | S200 | this | | | | | | | |
| | DSM50259 | | | study | | | | | | | |
| Pci S400/S200 | Pseudomonas | Phenol/ | GPC S400 & | Ranf/ | 200 | μg/mL | 0.13 | 50 | μg/mL | No | — |
| pool 4 | cichorii | water | DOC-GPC | Gisch, | | | | | | | |
| | ATCC10857/ | | S200 | this | | | | | | | |
| | DSM50259 | | | study | | | | | | | |

Heat-detergent-promoted LPS repurification

| Water control | — | — | — | — | — | | <LOQ | | | No | — |
|---|---|---|---|---|---|---|---|---|---|---|---|
| 3-OH—C10:0 | — | — | — | — | 20 | μM | 22.41 | 5 | μM | Yes | Yes |
| Pa H4 | Pseudomonas | PCP | | Sánchez | 100 | μg/mL | 5.38^{$} | 25 | μg/mL | Yes | Yes |
| | aeruginosa | | | Carballo | | | | | | | |
| | H4 | | | et al., | | | | | | | |
| | | | | 1999; | | | | | | | |
| | | | | Ranf | | | | | | | |
| | | | | et al., | | | | | | | |
| | | | | 2015 | | | | | | | |
| Repurified | Pseudomonas | PCP | Heat- | Ranf/ | 400 | μg/mL | <LOQ | 100 | μg/mL | No | — |
| Pa H4 | aeruginosa | | detergent | Gisch, | | | | | | | |
| | H4 | | repurifi- | this | | | | | | | |
| | | | cation | study | | | | | | | |

LPS of P. syringae pv. tomato DC3000 ΔpagL

| | | | | | | | | | | |
|---|---|---|---|---|---|---|---|---|---|---|
| Pst ΔpagL | Pseudomonas | 1) Phenol/ | Ranf, | 100 | μg/mL | 0.41^{$} | 20-50 | μg/mL | Yes | Yes |
| PP-sup sed | syringae pv. | water | this | | | | | | | |
| | tomato | 2) PCP | study | | | | | | | |
| | DC3000 | | | | | | | | | |
| | ΔpagL | | | | | | | | | |
| Pst ΔpagL | Pseudomonas | 1) Phenol/ | Ranf, | 100 | μg/mL | 0.33^{$} | 20-50 | μg/mL | Yes | Yes |
| PP-sed sed | syringae pv. | water | this | | | | | | | |
| | tomato | 2) PCP | study | | | | | | | |
| | DC3000 | | | | | | | | | |
| | ΔpagL | | | | | | | | | |

^{#}LPS preparations were generated using phenol/water extraction (Westphal and Jann, 1965), phenol-chloroform-petroleum ether (PCP) extraction (Galanos et al., 1969) or a combination of the two protocols in the indicated order.
^{$}Note that the total content of 3-OH—C10:0 in LPS samples is presumably significantly higher because only free 3-OH—C10:0 molecules (dissolved from LPS aggregates/micelles) but not the fraction still aggregated with LPS is considered under the assay conditions.

### FURTHER REFERENCES

Aalen R B. (2017). Plant Receptor Kinases. (New York, N.Y.: Springer New York).
Abdel-Mawgoud A M, Lépine F, and Déziel E. (2010a). Rhamnolipids: diversity of structures, microbial origins and roles 86: 1323-1336
Abdel-Mawgoud A M, Hausmann R, Lépine F, Müller M M, and Déziel E. (2010b). Rhamnolipids: Detection, Analysis, Biosynthesis, Genetic Regulation, and Bioengineering of Production (Berlin, Heidelberg: Springer Berlin Heidelberg), pp. 13-55.
Adamus G, Sikorska W, Janeczek H, Kwiecien M, Sobota M, and Kowalczuk M. (2012). Novel block copolymers of atactic PHB with natural PHA for cardiovascular engineering: Synthesis and characterization. European Polymer Journal 48: 621-631
Al-Shehbaz I A, Beilstein M A, and Kellogg E A. (2006). Systematics and phylogeny of the Brassicaceae (Cruciferae): an overview. Plant Systematics and Evolution 259: 89-120
Alberts B. (2017). Molecular biology of the cell.
Alexander C, and Rietschel E T. (2001). Bacterial lipopolysaccharides and innate immunity. J. Endotoxin Res. 7: 167-202
Aoyagi Y, Asakura R, Kondoh N, Yamamoto R, Kuromatsu T, Shimura A, and Ohta A. (1996). Samarium(II) Iodide Mediated Intermolecular Coupling Reactions of N,N-Dibenzyl-α-haloamides with Carbonyl Compounds. Synthesis 1996: 970-974
Asai T, Tena G, Plotnikova J, Willmann M R, Chiu W L, Gomez-Gomez L, Boller T, Ausubel F M, and Sheen J. (2002). MAP kinase signalling cascade in Arabidopsis innate immunity. Nature 415: 977-983
Ashby R, Solaiman D, and Foglia T. (2002). The synthesis of short- and medium-chain-length poly(hydroxyalkanoate) mixtures from glucose- or alkanoic acid-grown Pseudomonas oleovorans. Journal of Industrial Microbiology and Biotechnology 28: 147-153
Barbosa Pelegrini P, Del Sarto R P, Silva O N, Franco O L, and Grossi-de-Sa M F. (2011). Antibacterial peptides from plants: what they are and how they probably work. Biochem Res Int 2011: 250349
Bedini E, De Castro C, Erbs G, Mangoni L, Dow J M, Newman M A, Parrilli M, and Unverzagt C. (2005). Structure-dependent modulation of a pathogen response in plants by synthetic O-antigen polysaccharides. J. Am. Chem. Soc. 127: 2414-2416
Beyer E M, and Morgan P W. (1970). A method for determining the concentration of ethylene in the gas phase of vegetative plant tissues. Plant Physiol. 46: 352-354
Bitter W, van Boxtel R, Groeneweg M, Carballo P S, Zähringer U, Tommassen J, and Koster M. (2007). Species-specific functioning of the Pseudomonas XcpQ secretin: role for the C-terminal homology domain and lipopolysaccharide. Journal of bacteriology 189: 2967-2975
Bogino P C, Oliva Mde L, Sorroche F G, and Giordano W. (2013). The role of bacterial biofilms and surface components in plant-bacterial associations. Int J Mol Sci 14: 15838-15859
Boller T, and Felix G. (2009). A renaissance of elicitors: perception of microbe-associated molecular patterns and danger signals by pattern-recognition receptors. Annu Rev Plant Biol 60: 379-406
Botella J R, and Botella M A. (2016). Plant Signal Transduction. (New York, N.Y.: Springer New York).
Boudsocq M, Willmann M R, McCormack M, Lee H, Shan L, He P, Bush J, Cheng S H, and Sheen J. (2010). Differential innate immune signalling via Ca(2+) sensor protein kinases. Nature 464: 418-422
Breitenbach H H, Wenig M, Wittek F, Jorda L, Maldonado-Alconada A M, Sarioglu H, Colby T, Knappe C, Bichlmeier M, Pabst E, Mackey D, Parker J E, and Vlot A C. (2014). Contrasting Roles of the Apoplastic Aspartyl Protease APOPLASTIC, ENHANCED DISEASE SUSCEPTIBILITY1-DEPENDENT1 and LEGUME LECTIN-LIKE PROTEIN1 in Arabidopsis Systemic Acquired Resistance. Plant Physiol. 165: 791-809
Brelles-Mariño G. (2001). Detection, purification and characterisation of quorum-sensing signal molecules in plant-associated bacteria. Journal of Biotechnology 91: 197-209
Burke T R, Knight M, Chandrasekhar B, and Ferretti J A. (1989). Solid-phase synthesis of viscosin, a cyclic depsipeptide with antibacterial and antiviral properties. Tetrahedron Letters 30: 519-522
Bystrova O V, Shashkov A S, Kocharova N A, Knirel Y A, Lindner B, Zähringer U, and Pier G B. (2002). Structural studies on the core and the O-polysaccharide repeating unit of Pseudomonas aeruginosa immunotype 1 lipopolysaccharide. Eur J Biochem 269: 2194-2203
Chen H D, and Groisman E A. (2013). The biology of the PmrA/PmrB two-component system: the major regulator of lipopolysaccharide modifications. Annual review of microbiology 67: 83-112
Chung H S, and Sheen J. (2017). MAPK Assays in Arabidopsis MAMP-PRR Signal Transduction. Methods Mol Biol 1578: 155-166
Chung M G, Kim H W, Kim B R, Kim Y B, and Rhee Y H. (2012). Biocompatibility and antimicrobial activity of poly(3-hydroxyoctanoate) grafted with vinylimidazole. International Journal of Biological Macromolecules 50: 310-316
Concellón J M, and Concellón C. (2006). Aldol-type Reactions of Unmasked Iodoacetic Acid with Carbonyl Compounds Promoted by Samarium Diiodide: Efficient Synthesis of Carboxylic 3-Hydroxyacids and Their Derivatives. The Journal of Organic Chemistry 71:4428-4432
Coss C, Carrocci T, Maier R M, Pemberton J E, and Polt R. (2012). Minimally Competent Lewis Acid Catalysts: Indium(III) and Bismuth(III) Salts Produce Rhamnosides (=6-Deoxymannosides) in High Yield and Purity. Helvetica Chimica Acta 95: 2652-2659
Darbani B, Farajnia S, Toorchi M, Zakerbostanabad S, Noeparvar S, and Neal Stewart Jr C. (2008). DNA-Delivery Methods to Produce Transgenic Plants. Biotechnology(Faisalabad) 7: 385-402
Deng C, Zhang X, Zhang J, Qian J, and Zhu W. (2003). Rapid Determination of Salicylic Acid in Plant Materials by Gas Chromatography-Mass Spectrometry. Chromatographia 58: 225-229
Deng M-Z, Lu D-A, and Xu W-H. (1985). The reaction of trialkylvinylborate with carbon dioxide. A new method for the preparation of [small beta]-hydroxycarboxylic acids from alkenes. Journal of the Chemical Society, Chemical Communications 10.1039/C39850001478: 1478-1479
Dongari-Bagtzoglou A. (2008). Pathogenesis of mucosal biofilm infections: challenges and progress. Expert Rev Anti Infect Ther 6: 201-208
Dubiella U, Seybold H, Durian G, Komander E, Lassig R, Witte C P, Schulze W X, and Romeis T. (2013). Calcium-dependent protein kinase/NADPH oxidase activation circuit is required for rapid defense signal propagation. Proceedings of the National Academy of Sciences of the United States of America 110: 8744-8749
Duque P. (2016). Environmental Responses in Plants. (New York, N.Y.: Springer New York).
Duthaler R O, Herold P, Lottenbach W, Oertle K, and Riediker M. (1989). Enantioselektive Aldol-Reaktion von tert-Butylacetat mit Hilfe von Titan-Kohlenhydrat-Komplexen. Angewandte Chemie 101: 490-491
Ellis T N, Leiman S A, and Kuehn M J. (2010). Naturally produced outer membrane vesicles from Pseudomonas aeruginosa elicit a potent innate immune response via combined sensing of both lipopolysaccharide and protein components. Infection and Immunity 78: 3822-3831
Ernst R K, Adams K N, Moskowitz S M, Kraig G M, Kawasaki K, Stead C M, Trent M S, and Miller S I. (2006). The Pseudomonas aeruginosa lipid A deacylase: selection for expression and loss within the cystic fibrosis airway. J Bacteriol 188: 191-201
Felix G, Regenass M, and Boller T. (1993). Specific perception of subnanomolar concentrations of chitin fragments by tomato cells: induction of extracellular alkalinization, changes in protein phosphorylation, and establishment of a refractory state. The Plant Journal 4: 307-316
Felix G, Duran J D, Volko S, and Boller T. (1999). Plants have a sensitive perception system for the most conserved domain of bacterial flagellin. Plant J 18: 265-276
Fukuzawa S-i, Matsuzawa H, and Yoshimitsu S-i. (2000). Asymmetric Samarium-Reformatsky Reaction of Chiral a-Bromoacetyl-2-oxazolidinones with Aldehydes. The Journal of Organic Chemistry 65: 1702-1706
Galanos C, Luderitz O, and Westphal O. (1969). A new method for the extraction of R lipopolysaccharides. Eur J Biochem 9: 245-249
Galleano I, Schiedel M, Jung M, Madsen A S, and Olsen C A. (2016). A Continuous, Fluorogenic Sirtuin 2 Deacylase Assay: Substrate Screening and Inhibitor Evaluation. Journal of Medicinal Chemistry 59: 1021-1031
Geurtsen J, Steeghs L, Hove J T, van der Ley P, and Tommassen J. (2005). Dissemination of lipid A deacylases (pagL) among Gram-negative bacteria: identification of active-site histidine and serine residues. J Biol Chem 280: 8248-8259
Gomez-Gomez L, Felix G, and Boller T. (1999). A single locus determines sensitivity to bacterial flagellin in Arabidopsis thaliana. Plant J 18: 277-284
Green M R. (2012). Molecular cloning: a laboratory manual/Michael R. Green, Joseph Sambrook. (Cold Spring Harbor, N.Y: Cold Spring Harbor Laboratory Press).
Guo-Qiang C, Jun X, Qiong W, Zengming Z, and Kwok-Ping H. (2001). Synthesis of copolyesters consisting of medium-chain-length p-hydroxyalkanoates by Pseudomonas stutzeri 1317. Reactive and Functional Polymers 48: 107-112
Hase S, and Rietschel E T. (1977). The Chemical Structure of the Lipid A Component of Lipopolysaccharides from Chromobacterium violaceum NCTC 9694. European Journal of Biochemistry 75: 23-34
He P, Shan L, Lin N C, Martin G B, Kemmerling B, Nürnberger T, and Sheen J. (2006). Specific bacterial suppressors of MAMP signaling upstream of MAPKKK in Arabidopsis innate immunity. Cell 125: 563-575
Helmchen G, Leikauf U, and Taufer-Knöpfel I. (1985). Enantio- und anti-diastereoselektive Aldoladditionen von Acetaten und Propionaten über O-Silylketenacetale. Angewandte Chemie 97: 874-876
Hiroe A, Ishii N, Ishii D, Kabe T, Abe H, Iwata T, and Tsuge T. (2016). Uniformity of Monomer Composition and Material Properties of Medium-Chain-Length Polyhydroxyalkanoates Biosynthesized from Pure and Crude Fatty Acids. ACS Sustainable Chemistry & Engineering 4: 6905-6911
Hoang T T, Karkhoff-Schweizer R R, Kutchma A J, and Schweizer H P. (1998). A broad-host-range Flp-FRT recombination system for site-specific excision of chromosomally-located DNA sequences: application for isolation of unmarked Pseudomonas aeruginosa mutants. Gene 212: 77-86
Humphris S N, Cahill G, Elphinstone J G, Kelly R, Parkinson N M, Pritchard L, Toth I K, and Saddler G S. (2015). Detection of the Bacterial Potato Pathogens Pectobacterium and Dickeya spp. Using Conventional and Real-Time PCR. Methods Mol Biol 1302: 1-16
Huot B, Castroverde C D M, Velasquez A C, Hubbard E, Pulman J A, Yao J, Childs K L, Tsuda K, Montgomery B L, and He S Y. (2017). Dual impact of elevated temperature on plant defence and bacterial virulence in Arabidopsis. Nature Communications 8: 1808
Isshiki Y, Kawahara K, and Zähringer U. (1998). Isolation and characterisation of disodium (4-amino-4-deoxy-beta-L-arabinopyranosyl)-(1-->8)-(D-glycero-alpha-D-talo-oct-2-ulopyranosylonate)-(2-->4)-(methyl 3-deoxy-D-manno-oct-2-ulopyranosid)onate from the lipopolysaccharide of Burkholderia cepacia. Carbohydr. Res. 313: 21-27
Jaipuri F A, Francisca Jofre M, Schwarz K A, and Pohl N L. (2004). Microwave-assisted cleavage of Weinreb amide for carboxylate protection in the synthesis of a (R)-3-hydroxyalkanoic acid. Tetrahedron Letters 45: 4149-4152
Jimenez-Barbero J, Castro Cristina d, Evidente A, Molinaro A, Parrilli M, and Surico G. (2002). Structural Determination of the O-Specific Chain of the Lipopolysaccharide from Pseudomonas cichorii. European Journal of Organic Chemistry 2002: 1770-1775
Jin L, and Mackey D M. (2017). Measuring Callose Deposition, an Indicator of Cell Wall Reinforcement, During Bacterial Infection in Arabidopsis. Methods Mol Biol 1578: 195-205
Jones J D, and Dangl J L. (2006). The plant immune system. Nature 444: 323-329
Kadereit J W, Körner C, Kost B, and Sonnewald U. (2014). Strasburger—Lehrbuch der Pflanzenwissenschaften. (Berlin, Heidelberg: Springer Berlin Heidelberg).
Kalscheuer R, Arenskötter M, and Steinbüchel A. (1999). Establishment of a gene transfer system for Rhodococcus opacus PD630 based on electroporation and its application for recombinant biosynthesis of poly (3-hydroxyalkanoic acids). Applied microbiology and biotechnology 52: 508-515
Katagiri F, Thilmony R, and He S Y. (2002). The Arabidopsis thaliana-Pseudomonas syringae interaction. Arabidopsis Book 1: e0039
Kawahara K, Dejsirilert S, Danbara H, and Ezaki T. (1992). Extraction and characterization of lipopolysaccharide from Pseudomonas pseudomallei. FEMS Microbiol. Lett. 75: 129-133
Kieser K J, and Kagan J C. (2017). Multi-receptor detection of individual bacterial products by the innate immune system. Nat Rev Immunol 17: 376-390
Knight M R, Campbell A K, Smith S M, and Trewavas A J. (1991). Transgenic plant aequorin reports the effects of touch and cold-shock and elicitors on cytoplasmic calcium. Nature 352: 524-526
Knirel Y A, Zdorovenko G M, Paramonov N A, Veremeychenko S P, Toukach F V, and Shashkov A S. (1996). Somatic antigens of pseudomonads: structure of the O-specific polysaccharide of the reference strain for Pseudomonas fluorescens (IMV 4125, ATCC 13525, biovar A). Carbohydr. Res. 291: 217-224
Knirel Y A, Vinogradov E V, Shashkov A S, Dmitriev B A, Kochetkov N K, Stanislavsky E S, and Mashilova G M. (1985). Somatic antigens of Pseudomonas aeruginosa. The structure of the O-specific polysaccharide chains of lipopolysaccharides of P. aeruginosa serogroup O4 (Lanyi) and related serotype O6 (Habs) and immunotype 1 (Fisher). Eur J Biochem 150: 541-550
Kooistra O, Bedoux G, Brecker L, Lindner B, Sanchez Carballo P, Haras D, and Zähringer U. (2003). Structure of a highly phosphorylated lipopolysaccharide core in the Delta algC mutants derived from Pseudomonas aeruginosa wild-type strains PAO1 (serogroup O5) and PAC1R (serogroup O3). Carbohydr. Res. 338: 2667-2677
Kvitko B H, and Collmer A. (2011). Construction of Pseudomonas syringae pv. tomato DC3000 mutant and polymutant strains. Methods Mol Biol 712: 109-128
Lacomme C. (2015). Plant Pathology.
Lam J S, Taylor V L, Islam S T, Hao Y, and Kocincova D. (2011). Genetic and functional diversity of Pseudomonas aeruginosa lipopolysaccharide. Frontiers in microbiology 2: 118
Lavin J L, Kiil K, Resano O, Ussery D W, and Oguiza J A. (2007). Comparative genomic analysis of two-component regulatory proteins in Pseudomonas syringae. BMC Genomics 8: 397
Liang Z, Chen K, Zhang Y, Liu J, Yin K, Qiu J-L, and Gao C. (2018). Genome editing of bread wheat using biolistic delivery of CRISPR/Cas9 in vitro transcripts or ribonucleoproteins. Nature Protocols 13: 413
Lindeberg M, Cunnac S, and Collmer A. (2012). Pseudomonas syringae type Ill effector repertoires: last words in endless arguments. Trends in microbiology 20: 199-208
Liu Z Q, Mahmood T, and Yang P C. (2014). Western blot: technique, theory and trouble shooting. N Am J Med Sci 6: 160
Livak K J, and Schmittgen T D. (2001). Analysis of relative gene expression data using real-time quantitative PCR and the 2(−Delta Delta C(T)) Method. Methods 25: 402-408
Lloyd S R, Schoonbeek H J, Trick M, Zipfel C, and Ridout C J. (2014). Methods to study PAMP-triggered immunity in Brassica species. Molecular plant-microbe interactions: MPMI 27:286-295
Macho A P, and Zipfel C. (2014). Plant PRRs and the activation of innate immune signaling. Molecular cell 54: 263-272
Madala N E, Molinaro A, and Dubery I A. (2012). Distinct carbohydrate and lipid-based molecular patterns within lipopolysaccharides from Burkholderia cepacia contribute to defense-associated differential gene expression in Arabidopsis thaliana. Innate Immun. 18: 140-154
Mamat U, Schmidt H, Munoz E, Lindner B, Fukase K, Hanuszkiewicz A, Wu J, Meredith T C, Woodard R W, Hilgenfeld R, Mesters J R, and Holst O. (2009). WaaA of the hyperthermophilic bacterium Aquifex aeolicus is a monofunctional 3-deoxy-D-manno-oct-2-ulosonic acid transferase involved in lipopolysaccharide biosynthesis. J Biol Chem 284: 22248-22262
Masoud H, Lindner B, Weckesser J, and Mayer H. (1990). The Structure of the Lipid A Component of Rhodocyclus gelatinosus Dr2 Lipopolysaccharide. Systematic and Applied Microbiology 13: 227-233
Masoud H, Altman E, Richards J C, and Lam J S. (1994). General strategy for structural analysis of the oligosaccharide region of lipooligosaccharides. Structure of the oligosaccharide component of Pseudomonas aeruginosa IATS serotype 06 mutant R5 rough-type lipopolysaccharide. Biochemistry 33: 10568-10578
Matsumoto Ki, Nakae S, Taguchi K, Matsusaki H, Seki M, and Doi Y. (2001). Biosynthesis of Poly(3-hydroxybutyrate-co-3-hydroxyalkanoates) Copolymer from Sugars by Recombinant Ralstonia eutropha Harboring the phaC1Ps and the phaGPs Genes of Pseudomonas sp. 61-3. Biomacromolecules 2: 934-939
McDowell J M. (2011). Plant Immunity. (Totowa, N.J.: Humana Press).
Miller G, Schlauch K, Tam R, Cortes D, Torres M A, Shulaev V, Dangl J L, and Mittler R. (2009). The plant NADPH oxidase RBOHD mediates rapid systemic signaling in response to diverse stimuli. Sci Signal 2: ra45
Miller S I, Ernst R K, and Bader M W. (2005). LPS, TLR4 and infectious disease diversity. Nat Rev Microbiol 3: 36-46
Mishina T E, and Zeier J. (2007). Pathogen-associated molecular pattern recognition rather than development of tissue necrosis contributes to bacterial induction of systemic acquired resistance in Arabidopsis. Plant J 50: 500-513
Molinaro A, Newman M-A, Lanzetta R, and Parrilli M. (2009). The Structures of Lipopolysaccharides from Plant-Associated Gram-Negative Bacteria. European Journal of Organic Chemistry 2009: 5887-5896
Mott G A, Thakur S, Smakowska E, Wang P W, Belkhadir Y, Desveaux D, and Guttman D S. (2016). Genomic screens identify a new phytobacterial microbe-associated molecular pattern and the cognate Arabidopsis receptor-like kinase that mediates its immune elicitation. Genome Biol 17: 98
Mott G A, Desveaux D, and Guttman D S. (2018). A high-sensitivity, microtiter-based plate assay for plant pattern-triggered immunity. Mol Plant Microbe Interact 31: 499-504
Nakahata M, Imaida M, Ozaki H, Harada T, and Tai A. (1982). The preparation of optically pure 3-hydroxyalkanoic acid. The enantioface-differentiating hydrogenation of the C═O double bond with modified Raney nickel. XXXVII. Bulletin of the Chemical Society of Japan 55: 2186-2189
Needham B D, and Trent M S. (2013). Fortifying the barrier: the impact of lipid A remodelling on bacterial pathogenesis. Nat Rev Microbiol 11: 467-481
Newman M A, Sundelin T, Nielsen J T, and Erbs G. (2013). MAMP (microbe-associated molecular pattern) triggered immunity in plants. Front. Plant. Sci. 4: 139
Nguyen H P, Chakravarthy S, Velasquez A C, McLane H L, Zeng L, Nakayashiki H, Park D H, Collmer A, and Martin G B. (2010). Methods to study PAMP-triggered immunity using tomato and Nicotiana benthamiana. Molecular plant-microbe interactions: MPMI 23: 991-999
Norris A T, and Bloch K. (1963). On the Mechanism of the Enzymatic Synthesis of Unsaturated Fatty Acids in Escherichia coli. Journal of Biological Chemistry 238: PC3133-PC3134
Pallas V, Sanchez-Navarro J, Varga A, Aparicio F, and James D. (2009). Multiplex polymerase chain reaction (PCR) and real-time multiplex PCR for the simultaneous detection of plant viruses. Methods Mol Biol 508: 193-208
Park B S, Song D H, Kim H M, Choi B S, Lee H, and Lee J O. (2009). The structural basis of lipopolysaccharide recognition by the TLR4-MD-2 complex. Nature 458: 1191-1195
Park S J, Lee S H, Oh Y H, and Lee S Y. (2015). Establishment of a biosynthesis pathway for (R)-3-hydroxyalkanoates in recombinant Escherichia coli. Korean Journal of Chemical Engineering 32: 702-706
Peprah B A, Ramsay J A, and Ramsay B A. (2016). Dense stable suspensions of medium-chain-length poly(3-hydroxyalkanoate) nanoparticles. European Polymer Journal 84: 137-146
Peters J L, Cnudde F, and Gerats T. (2003). Forward genetics and map-based cloning approaches. Trends Plant Sci 8: 484-491
Peterson A A, and McGroarty E J. (1985). High-molecular-weight components in lipopolysaccharides of Salmonella typhimurium, Salmonella minnesota, and Escherichia coli. Journal of bacteriology 162: 738-745
Phithakrotchanakoon C, Champreda V, Aiba S-i, Pootanakit K, and Tanapongpipat S. (2013). Engineered Escherichia coli for Short-Chain-Length Medium-Chain-Length Polyhydroxyalkanoate Copolymer Biosynthesis from Glycerol and Dodecanoate. Bioscience, Biotechnology, and Biochemistry 77: 1262-1268
Pieterse C M, Van der Does D, Zamioudis C, Leon-Reyes A, and Van Wees S C. (2012). Hormonal modulation of plant immunity. Annual review of cell and developmental biology 28: 489-521
Poirier Y, Erard N, and MacDonald-Comber Petétot J. (2002). Synthesis of polyhydroxyalkanoate in the peroxisome of Pichia pastoris. FEMS Microbiology Letters 207: 97-102
Raaijmakers J M, De Bruijn I, and de Kock M J D. (2006). Cyclic lipopeptide production by plant-associated Pseudomonas spp.: diversity, activity, biosynthesis, and regulation. Molecular Plant-Microbe Interactions 19: 699-710
Raaijmakers J M, De Bruijn I, Nybroe O, and Ongena M. (2010). Natural functions of lipopeptides from Bacillus and Pseudomonas: more than surfactants and antibiotics. FEMS Microbiol Rev 34:1037-1062
Raetz C R, Reynolds C M, Trent M S, and Bishop R E. (2007). Lipid A modification systems in gram-negative bacteria. Annu Rev Biochem 76: 295-329
Raetz C R H, and Whitfield C. (2002). Lipopolysaccharide endotoxins. Annu. Rev. Biochem. 71: 635-700
Ranf S. (2017). Sensing of molecular patterns through cell surface immune receptors. Current Opinion in Plant Biology 38: 68-77
Ranf S, Eschen-Lippold L, Pecher P, Lee J, and Scheel D. (2011). Interplay between calcium signalling and early signalling elements during defence responses to microbe- or damage-associated molecular patterns. Plant Journal 68: 100-113
Ranf S, Grimmer J, Poeschl Y, Pecher P, Chinchilla D, Scheel D, and Lee J. (2012). Defense-Related Calcium Signaling Mutants Uncovered via a Quantitative High-Throughput Screen in Arabidopsis thaliana. Molecular Plant 5: 115-130
Ranf S, Wuennenberg P, Lee J, Becker D, Dunkel M, Hedrich R, Scheel D, and Dietrich P. (2008). Loss of the vacuolar cation channel, AtTPC1, does not impair Ca2+ signals induced by abiotic and biotic stresses. Plant Journal 53: 287-299
Ranf S, Gisch N, Schaeffer M, Illig T, Westphal L, Knirel Y A, Sanchez-Carballo P M, Zaehringer U, Hueckelhoven R, Lee J, and Scheel D. (2015). A lectin S-domain receptor kinase mediates lipopolysaccharide sensing in Arabidopsis thaliana. Nature Immunology 16: 426-433
Ronald P C. (2006). Plant-Pathogen Interactions. (New Jersey: Humana Press).
Roo Gd, Kellerhals M B, Ren Q, Witholt B, and Kessler B. (2002). Production of chiral R-3-hydroxyalkanoic acids and R-3-hydroxyalkanoic acid methylesters via hydrolytic degradation of polyhydroxyalkanoate synthesized by pseudomonads. Biotechnology and Bioengineering 77: 717-722
Sailer M, Dubicki K I, and Sorensen J L. (2015). The Synthesis of Medium-Chain-Length p-Hydroxy Esters via the Reformatsky Reaction. Synthesis 47: 79-82
Sánchez Carballo P M, Rietschel E T, Kosma P, and Zähringer U. (1999). Elucidation of the structure of an alanine-lacking core tetrasaccharide trisphosphate from the lipopolysaccharide of Pseudomonas aeruginosa mutant H4. Eur J Biochem 261: 500-508
Sánchez R J, Schripsema J, da Silva L F, Taciro M K, Pradella J G C, and Gomez J G C. (2003). Medium-chain-length polyhydroxyalkanoic acids (PHAmcl) produced by Pseudomonas putida IPT 046 from renewable sources. European Polymer Journal 39: 1385-1394
Sanguanchaipaiwong V, Gabelish C L, Hook J, Scholz C, and Foster L J R. (2004). Biosynthesis of Natural-Synthetic Hybrid Copolymers: Polyhydroxyoctanoate-Diethylene Glycol. Biomacromolecules 5: 643-649
Sato S, Ishii N, Hamada Y, Abe H, and Tsuge T. (2012). Utilization of 2-alkenoic acids for biosynthesis of medium-chain-length polyhydroxyalkanoates in metabolically engineered Escherichia coli to construct a novel chemical recycling system. Polymer Degradation and Stability 97: 329-336
Schoonbeek H J, Wang H H, Stefanato F L, Craze M, Bowden S, Wallington E, Zipfel C, and Ridout C J. (2015). Arabidopsis EF-Tu receptor enhances bacterial disease resistance in transgenic wheat. The New phytologist 206: 606-613
Schroeder T H, Lee M M, Yacono P W, Cannon C L, Gerceker A A, Golan D E, and Pier G B. (2002). CFTR is a pattern recognition molecule that extracts Pseudomonas aeruginosa LPS from the outer membrane into epithelial cells and activates NF-kappa B translocation. Proceedings of the National Academy of Sciences of the United States of America 99: 6907-6912
Schwechheimer C, and Kuehn M J. (2015). Outer-membrane vesicles from Gram-negative bacteria: biogenesis and functions. Nat Rev Microbiol 13: 605-619
Senchenkova S N, Shashkov A S, Laux P, Knirel Y A, and Rudolph K. (1999). The O-chain polysaccharide of the lipopolysaccharide of Xanthomonas campestris pv. begoniae GSPB 525 is a partially L-xylosylated L-rhamnan. Carbohydr. Res. 319: 148-153
Senchenkova S N, Huang X, Laux P, Knirel Y A, Shashkov A S, and Rudolph K. (2002). Structures of the O-polysaccharide chains of the lipopolysaccharides of Xanthomonas campestris pv phaseoli var fuscans GSPB 271 and X. campestris pv malvacearum GSPB 1386 and GSPB 2388. Carbohydr. Res. 337: 1723-1728
Seybold H, Boudsocq M, and Romeis T. (2017). CDPK Activation in PRR Signaling. Methods Mol Biol 1578: 173-183
Seydel U, Oikawa M, Fukase K, Kusumoto S, and Brandenburg K. (2000). Intrinsic conformation of lipid A is responsible for agonistic and antagonistic activity. Eur J Biochem 267: 3032-3039
Shan L, and He P. (2017). Plant Pattern Recognition Receptors. (New York, N.Y.: Springer New York).
Shang-Guan K, Wang M, Htwe N, Li P, Li Y, Qi F, Zhang D, Cao M, Kim C, Weng H, Cen H, Black I M, Azadi P, Carlson R W, Stacey G, and Liang Y. (2018). Lipopolysaccharides trigger two successive bursts of reactive oxygen species at distinct cellular locations. Plant Physiol 176: 2543-2556
Shi J, Zhao Y, Wang Y, Gao W, Ding J, Li P, Hu L, and Shao F. (2014). Inflammatory caspases are innate immune receptors for intracellular LPS. Nature 514: 187-192
Shimomura O, Johnson F H, and Saiga Y. (1962). Extraction, purification and properties of aequorin, a bioluminescent protein from the luminous hydromedusan, Aequorea. J Cell Comp Physiol 59: 223-239
Shiu S, and Bleecker A. (2003). Expansion of the receptor-like kinase/Pelle gene family and receptor-like proteins in Arabidopsis. Plant Physiol. 132: 530-543
Silipo A, Molinaro A, Sturiale L, Dow J M, Erbs G, Lanzetta R, Newman M A, and Parrilli M. (2005). The elicitation of plant innate immunity by lipooligosaccharide of Xanthomonas campestris. J Biol Chem 280: 33660-33668
Solaiman D K Y, Ashby R D, and Foglia T A. (2002). Physiological Characterization and Genetic Engineering of Pseudomonas corrugata for Medium-Chain-Length Polyhydroxyalkanoates Synthesis from Triacylglycerols. Current Microbiology 44: 189-195
Spoel S H, and Dong X. (2012). How do plants achieve immunity? Defence without specialized immune cells. Nat Rev Immunol 12: 89-100
Tahara S, and Mizutani J. (1978). Preparation of L-3-Hydoxyalkanoic Acids by Fungal Hydration of the Corresponding trans-2-Alkenoic Acids. Agricultural and Biological Chemistry 42: 879-883
Tan Y, and Kagan J C. (2014). A cross-disciplinary perspective on the innate immune responses to bacterial lipopolysaccharide. Molecular cell 54: 212-223
Tappel R C, Wang Q, and Nomura C T. (2012a). Precise control of repeating unit composition in biodegradable poly(3-hydroxyalkanoate) polymers synthesized by Escherichia coli. Journal of Bioscience and Bioengineering 113: 480-486
Tappel R C, Kucharski J M, Mastroianni J M, Stipanovic A J, and Nomura C T. (2012b). Biosynthesis of Poly[(R)-3-hydroxyalkanoate] Copolymers with Controlled Repeating Unit Compositions and Physical Properties. Biomacromolecules 13: 2964-2972
Tappel R C, Pan W, Bergey N S, Wang Q, Patterson I L, Ozumba O A, Matsumoto Ki, Taguchi S, and Nomura C T. (2014). Engineering Escherichia coli for Improved Production of Short-Chain-Length-co-Medium-Chain-Length Poly[(R)-3-hydroxyalkanoate] (SCL-co-MCL PHA) Copolymers from Renewable Nonfatty Acid Feedstocks. ACS Sustainable Chemistry & Engineering 2: 1879-1887
Teixidó M, Caba J M, Prades R, Zurita E, Martinell M, Vilaseca M, Albericio F, and Giralt E. (2007). Does the Solid-Phase Synthesis of a Tetrapeptide Represent a Challenge at the Onset of the XXI Century? The Case of Cyclo [(3R)-3-hydroxydecanoyl-1-seryl-(3R)-3-hydroxydecanoyl-I-seryl]. International Journal of Peptide Research and Therapeutics 13: 313-327
Tena G, Boudsocq M, and Sheen J. (2011). Protein kinase signaling networks in plant innate immunity. Curr Opin Plant Biol 14: 519-529
Tirsoaga A, Novikov A, Adib-Conquy M, Werts C, Fitting C, Cavaillon J-M, and Caroff M. (2007). Simple Method for Repurification of Endotoxins for Biological Use. Applied and Environmental Microbiology 73: 1803-1808
Trujillo M. (2016). Analysis of the Immunity-Related Oxidative Bursts by a Luminol-Based Assay. Methods Mol Biol 1398: 323-329
Tsai C-C, Chuang W-T, Tsai Y-F, Li J-T, Wu Y-F, and Liao C-C. (2013). Intra- and intermolecular hydrogen bonds enhance the fluoride-responsiveness of functionalized glycolipid-based gelators. Journal of Materials Chemistry B 1: 819-827
Vallarino J G, and Osorio S. (2016). Simultaneous Determination of Plant Hormones by GC-TOF-MS. Methods Mol Biol 1363: 229-237
Van Loon L C, and Van Strien E A. (1999). The families of pathogenesis-related proteins, their activities, and comparative analysis of PR-1 type proteins. Physiological and Molecular Plant Pathology 55: 85-97
Verlinden R A, Hill D J, Kenward M A, Williams C D, and Radecka I. (2007). Bacterial synthesis of biodegradable polyhydroxyalkanoates. J Appl Microbiol 102: 1437-1449
Vleeschouwer M D, Sinnaeve D, Begin JVd, Coenye T, Martins J C, and Madder A. (2014). Rapid Total Synthesis of Cyclic Lipodepsipeptides as a Premise to Investigate their Self-Assembly and Biological Activity. Chemistry—A European Journal 20: 7766-7775
Vlot A C, Liu P P, Cameron R K, Park S W, Yang Y, Kumar D, Zhou F, Padukkavidana T, Gustafsson C, Pichersky E, and Klessig D F. (2008). Identification of likely orthologs of tobacco salicylic acid-binding protein 2 and their role in systemic acquired resistance in Arabidopsis thaliana. Plant J 56: 445-456
Voinnet O, Rivas S, Mestre P, and Baulcombe D. (2003). An enhanced transient expression system in plants based on suppression of gene silencing by the p19 protein of tomato bushy stunt virus. Plant J 33: 949-956
Wang H-h, Zhou X-r, Liu Q, and Chen G-Q. (2011). Biosynthesis of polyhydroxyalkanoate homopolymers by Pseudomonas putida. Applied Microbiology and Biotechnology 89: 1497-1507
Wang Q, Tappel R C, Zhu C, and Nomura C T. (2012). Development of a New Strategy for Production of Medium-Chain-Length Polyhydroxyalkanoates by Recombinant Escherichia coli via Inexpensive Non-Fatty Acid Feedstocks. Applied and Environmental Microbiology 78: 519-527
Ward P G, and O'Connor K E. (2005). Bacterial synthesis of polyhydroxyalkanoates containing aromatic and aliphatic monomers by Pseudomonas putida CA-3. International Journal of Biological Macromolecules 35: 127-133
Weis C, Huckelhoven R, and Eichmann R. (2013). LIFEGUARD proteins support plant colonization by biotrophic powdery mildew fungi. J Exp Bot 64: 3855-3867
Westphal O, and Jann K. (1965). Bacterial lipopolysaccharide. Extraction with phenol-water and further applications of the procedure. In Methods Carbohydr. Chem., R Whistler and M Wolfan, eds (New York: Academic press), pp. 83-91.
Whitfield C, and Trent M S. (2014). Biosynthesis and export of bacterial lipopolysaccharides. Annu Rev Biochem 83: 99-128
Wilkinson M D. (2005). Methods in molecular biology, volume 286. Transgenic plants. Methods and protocols. Peña L, ed. 2005. Totowa, N.J.: Humana Press. $115 (hardback). 448 pp. Annals of Botany 95: 1068-1068
Wise A A, Liu Z, and Binns A N. (2006). Culture and maintenance of Agrobacterium strains. Methods Mol Biol 343: 3-13
Wollenweber H W, Seydel U, Lindner B, Luderitz O, and Rietschel E T. (1984). Nature and location of amide-bound (R)-3-acyloxyacyl groups in lipid A of lipopolysaccharides from various gram-negative bacteria. Eur J Biochem 145: 265-272
Wu C, Miller P A, and Miller M J. (2011). Syntheses and studies of amamistatin B analogs reveals that anticancer activity is relatively independent of stereochemistry, ester or amide linkage and select replacement of one of the metal chelating groups. Bioorganic & Medicinal Chemistry Letters 21: 2611-2615
Wu Q, Zheng Z, Xi J-Z, Gao H, and Chen G-Q. (2003). Production of Hydroxyalkanoate Monomers by Microbial Fermentation. JOURNAL OF CHEMICAL ENGINEERING OF JAPAN 36:1170-1173
Yin K, Gao C, and Qiu J L. (2017). Progress and prospects in plant genome editing. Nat Plants 3: 17107
Zähringer U, Lindner B, and Rietschel E T. (1994). Molecular structure of lipid A, the endotoxic center of bacterial lipopolysaccharides. Adv Carbohydr Chem Biochem 50: 211-276
Zeidler D, Zähringer U, Gerber I, Dubery I, Hartung T, Bors W, Hutzler P, and Durner J. (2004). Innate immunity in Arabidopsis thaliana: Lipopolysaccharides activate nitric oxide synthase (NOS) and induce defense genes. Proceedings of the National Academy of Sciences of the United States of America 101: 15811-15816
Zhang K, Raboanatahiry N, Zhu B, and Li M. (2017). Progress in Genome Editing Technology and Its Application in Plants. Front. Plant. Sci. 8: 177
Zheng Z, Gong Q, and Chen G. (2004). A novel method for production of 3-hydroxydecanoic acid by recombinant Escherichia coli and Pseudomonas putida. Chinese Journal of Chemical Engineering 12: 550-555
Zipfel C, and Felix G. (2005). Plants and animals: a different taste for microbes? Curr Opin Plant Biol 8: 353-360

What is claimed is:

1. A method for determining whether a plant expresses the LipoOligosaccharid-specific Reduced Elicitation (LORE) represented by SEQ ID NO:1, or a functional variant thereof capable of activating pattern-triggered immunity (PTI), the method comprising the steps of:
(a) contacting the plant, or a part thereof, with a compound of formula (I): 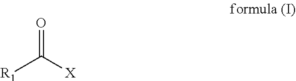
or a salt thereof,
wherein
R₁ is selected from —OH, —H, —OCH₃, —OCH₂CH₃,
—O—(CH₂)₂₋₁₁—CH₃, wherein one hydrogen atom in the —(CH₂)₂₋₁₁— group may be replaced by a —CH₃ group,
—O—(CH₂)₁₋₁₂—OH, wherein one or two hydrogen atoms in the —(CH₂)₁₋₁₂— group may be replaced by a —CH₃ group,
—O—(CH₂)₁₋₁₁—COOH, wherein one hydrogen atom in the —(CH₂)₁₋₁₁— group may be replaced by a —CH₃ group,
—NH₂, —SH, —SCH₃, —SCH₂CH₃,
an amino acid residue which is attached via an amino group to form an amide bond with the carbonyl group of formula (I) and wherein the carboxyl group of the amino acid residue may be converted into an ester group, preferably a C₁-C₆ alkyl ester, more preferably a methyl ester, and
a biogenic amine which is attached via an amino group to form an amide bond with the carbonyl group of formula (I);
and X is selected from one of formulas (II) and (III): 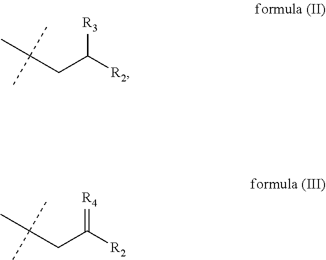
wherein
R₂ is —(CH₂)₄₋₈—CH₃;
R₃ is selected from —OH, —SH, —OCOCH₂CHOH(CH₂)₄₋₈CH₃; and
R₄ is selected from ═O, ═S, ═NH, and ═CH₂;
and the dashed line in formula (II) and (III) marks the bond which attaches X to the remainder of the compound of formula (I)
and, subsequently,
(b) determining whether PTI is activated,
wherein the activation of PTI indicates that the plant expresses the functional LORE, or a functional variant thereof.

2. The method of claim 1, wherein
R₃ is —OH or —SH, and
R₄ is ═O or ═S.

3. The method of claim 1 or 2, wherein R₁ is selected from —OH, —OCH₃, —OCH₂CH₃, —O(CH₂)₂₋₃—CH₃, —SH, —NH₂, and —NH—CH₂—COOH.

4. The method of any one of claims 1 to 3, wherein the compound of formula (I) is selected from
3-hydroxydecanoic acid
3-hydroxyundecanoic acid
3-hydroxynonanoic acid
3-hydroxydodecanoic acid
3-hydroxyoctanoic acid
methyl-3-hydroxydecanoate
methyl-3-hydroxyundecanoate
methyl-3-hydroxynonanoate
methyl-3-hydroxydodecanoate
methyl-3-hydroxyoctanoate
ethyl-3-hydroxydecanoate
ethyl-3-hydroxyundecanoate
ethyl-3-hydroxynonanoate
(3-hydroxydecanoyl)glycine
(3-hydroxyundecanoyl)glycine
(3-hydroxynonanoyl)glycine
methyl (3-hydroxydecanoyl)glycinate
methyl (3-hydroxyundecanoyl)glycinate
methyl (3-hydroxynonanoyl)glycinate
3-oxo-decanoic acid
3-oxo-undecanoic acid
3-oxo-nonanoic acid
3-(hydroxydecanoyloxy)decanoic acid
3-(hydroxydecanoyloxy)dodecanoic acid
3-(hydroxydodecanoyloxy)dodecanoic acid
3-(hydroxyddecanoyloxy)decanoic acid
(3-hydroxydecanoyl)-L-leucine
propyl-3-hydroxydecanoic acid
butyl-3-hydroxydecanoic acid.

5. The method of any one of claims 1 to 4, wherein the plant is contacted with the compound of formula (I) in an amount of 1 nM to 1 mM.

6. The method of any one of claims 1 to 5, wherein the contacting of the plant with the compound of formula (I) is by spraying, dusting, scattering, coating and pouring.

7. The method according to any one of claims 1 to 6, wherein the plant or part thereof is selected from: seedlings, leaf discs, leaves, stems, branches, roots, whole plants, cells, protoplasts, flowers and fruits.

8. The method according to any one of claims 1 to 7, wherein the functional variant of LORE is a naturally occurring or gene-technologically modified LORE mutant, a LORE-homologue or a LORE-analogue.

9. The method according to any one of claims 1 to 8, wherein the plant is a plant of the family Brassicaceae.

10. A screening method for identifying functional variants of LORE capable of activating pattern-triggered immunity (PTI) represented by SEQ ID NO:1, the method comprising the steps of:
(a) determining whether one or more plant(s) express(es) LORE as represented by SEQ ID NO:1, or a functional variant thereof, by the method of any one of claims 1 to 9;
(b) determining the amino acid sequence of the LORE or the functional variant thereof in the plants identified in (a); and
(c) comparing the amino acid sequence determined in (b) with the amino acid sequence of LORE represented by SEQ ID NO:1, wherein any amino acid sequence that differs from the amino acid sequence of SEQ ID NO:1 encodes a functional variant of LORE.

11. A method of inducing pattern-triggered immunity (PTI) in a plant, the method comprising:
(a) contacting a plant that expresses LORE represented by SEQ ID NO:1, or a functional variant thereof capable of activating PTI, or a part of said plant, with a compound of formula (I): 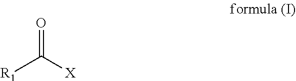
or a salt thereof,
wherein
R₁ is selected from —OH, —H, —OCH₃, —OCH₂CH₃, —O—(CH₂)₂₋₁₁—CH₃, wherein one hydrogen atom in the —(CH₂)₂₋₁₁— group may be replaced by a —CH₃ group,
—O—(CH₂)₁₋₁₂—OH, wherein one or two hydrogen atoms in the —(CH₂)₁₋₁₂— group may be replaced by a —CH₃ group,
—O—(CH₂)₁₋₁₁—COOH, wherein one hydrogen atom in the —(CH₂)₁₋₁₁— group may be replaced by a —CH₃ group,
—NH₂, —SH, —SCH₃, —SCH₂CH₃,
an amino acid residue which is attached via an amino group to form an amide bond with the carbonyl group of formula (I) and wherein the carboxyl group of the amino acid residue may be converted into an ester group, preferably a CC alkyl ester, more preferably a methyl ester, and
a biogenic amine which is attached via an amino group to form an amide bond with the carbonyl group of formula (I);
and X is selected from one of formulas (II) and (III): 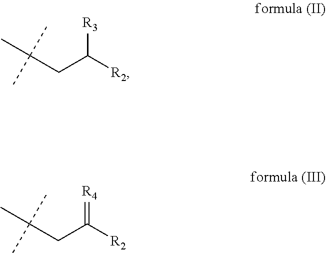
wherein
R₂ is —(CH₂)₄₋₈—CH₃;
R₃ is selected from —OH, —SH, —OCOCH₂CHOH(CH₂)₄₋₈CH₃; and
R₄ is selected from ═O, ═S, ═NH, and ═CH₂;
and the dashed line in formula (II) and (III) marks the bond which attaches X to the remainder of the compound of formula (I).

12. The method of claim 11, further comprising the step:
(a-0) modifying a plant to express LORE represented by SEQ ID NO:1, or a functional variant thereof capable of activating PTI.

13. The method of claim 12, wherein the functional variant of LORE capable of activating PTI is a functional variant of LORE identified by the method of claim 10 or 11.

14. The method of claim 12 or 13, wherein the modifying is by genetical engineering.

15. A plant protective composition comprising or consisting of a compound of formula (I): 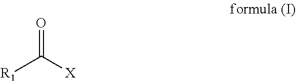
or a salt thereof,
wherein
R₁ is selected from —OH, —H, —OCH₃, —OCH₂CH₃,
—O—(CH₂)₂₋₁₁—CH₃, wherein one hydrogen atom in the —(CH₂)₂₋₁₁— group may be replaced by a —CH₃ group,
—O—(CH₂)₁₋₁₂—OH, wherein one or two hydrogen atoms in the —(CH₂)₁₋₁₂— group may be replaced by a —CH₃ group,
—O—(CH₂)₁₋₁₁—COOH, wherein one hydrogen atom in the —(CH₂)₁₋₁₁— group may be replaced by a —CH₃ group,
—NH₂, —SH, —SCH₃, —SCH₂CH₃,
an amino acid residue which is attached via an amino group to form an amide bond with the carbonyl group of formula (I) and wherein the carboxyl group of the amino acid residue may be converted into an ester group, preferably a C₁-C₆ alkyl ester, more preferably a methyl ester, and
a biogenic amine which is attached via an amino group to form an amide bond with the carbonyl group of formula (I);
and X is selected from one of formulas (II) and (III): 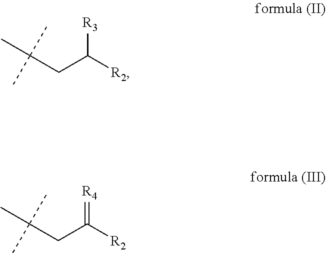
wherein
R₂ is —(CH₂)₄₋₈—CH₃;
R₃ is selected from —OH, —SH, —OCOCH₂CHOH(CH₂)₄₋₈CH₃; and
R₄ is selected from ═O, ═S, ═NH, and ═CH₂;
and the dashed line in formula (II) and (III) marks the bond which attaches X to the remainder of the compound of formula (I)
optionally in combination with (a) carrier(s) and/or additive(s).


SEQUENCE LISTING (2 sequences)

SEQ ID NO: 1 - protein, 805 aa, Arabidopsis thaliana
  LORE Protein AT1G61380.1
MGMVLFACLLLLIIFPTCGYAAINTSSPLSIRQTLSSPGGFYELGFFSPNNTQNQYVGIWFKKIVPRVVVWVANRDTPVTSSAANLTISSNGSLILLDGKQDVIWSTGKAFTSNKCHAELLDTGNFVVIDDVSGNKLWQSFEHLGNTMLPQSSLMYDTSNGKKRVLTTWKSNSDPSPGEFSLEITPQIPTQGLIRRGSVPYWRCGPWAKTRFSGISGIDASYVSPFSVVQDTAAGTGSFSYSTLRNYNLSYVTLTPEGKMKILWDDGNNWKLHLSLPENPCDLYGRCGPYGLCVRSDPPKCECLKGFVPKSDEEWGKGNWTSGCVRRTKLSCQAKSSMKTQGKDTDIFYRMTDVKTPDLHQFASFLNAEQCYQGCLGNCSCTAFAYISGIGCLVWNGELADTVQFLSSGEFLFIRLASSELAGSSRRKIIVGTTVSLSIFLILVFAAIMLWRYRAKQNDAWKNGFERQDVSGVNFFEMHTIRTATNNFSPSNKLGQGGFGPVYKGKLVDGKEIGVKRLASSSGQGTEEFMNEITLISKLQHRNLVRLLGYCIDGEEKLLIYEFMVNKSLDIFIFDPCLKFELDWPKRFNIIQGIARGLLYLHRDSRLRVIHRDLKVSNILLDDRMNPKISDFGLARMFQGTQYQDNTRRVVGTLGYMSPEYAWAGLFSEKSDIYSFGVLMLEIISGKRISRFIYGDESKGLLAYTWDSWCETGGSNLLDRDLTDTCQAFEVARCVQIGLLCVQHEAVDRPNTLQVLSMLTSATDLPVPKQPIFAVHTLNDMPMLQANSQDFLSVNEMTESMIQGR

SEQ ID NO: 2 - DNA, 2418 bp, Arabidopsis thaliana
  LORE DNA coding sequence
atgggtatggttttatttgcttgcttgcttttactaatcatattcccaacttgtggctatgcagctataaacacatcaagtcctttgtcaataagacaaactctgagttctcctggtggattttatgagttaggcttcttcagtcctaataatactcagaatcagtatgtcgggatctggttcaagaagattgttcctcgtgtagttgtgtgggtggctaacagagatacgccagttactagctctgcagcaaatctcactatcagcagcaatgggagcctgatcttgcttgatgggaaacaagatgttatttggtcaacaggaaaagctttcacatccaacaaatgtcatgcggagctcttagataccggaaattttgttgtaattgatgatgtttcagggaataaattatggcaaagcttcgagcatcttggtaatactatgctgcctcagtcatcattgatgtatgatacttctaatggcaagaagcgtgtgttgactacatggaaaagtaacagcgatccttcgcctggggagttctcactggagattacaccacaaatccccacacaaggccttataagaagaggctcagtaccttactggagatgtggtccttgggcaaaaacaagattctctgggatatctggaattgatgcatcatatgtaagtccattcagcgttgtccaggatacagcagccggcacaggatctttctcttattccacattaagaaactataatctctcatatgttacgctaacaccagaagggaaaatgaagatcctctgggatgatggaaataattggaagcttcacttgtctctcccagagaacccatgtgatctatatggtagatgcgggccttatggtttgtgtgtgagatctgatcccccaaagtgtgaatgcttgaaagggtttgtaccaaagtctgatgaggagtggggaaaaggaaactggacaagtgggtgtgtgagacgtacaaaattatcttgccaagcaaaatcttctatgaaaacacaaggcaaagacacagacatcttctatcgtatgacagatgtaaagactccggatctgcaccaatttgcaagctttctcaatgccgaacagtgctatcaaggttgtctaggcaactgttcttgcacagccttcgcctatattagtggaattggatgtttggtatggaatggggagctagcagacaccgttcaatttttgtctagcggagagtttctcttcattcgtcttgcaagttcagaattggctggaagcagtcgaaggaagattatcgtaggcactactgtcagcctttccattttcttgatcttggtatttgctgcaattatgttgtggagatacagagcgaaacaaaatgatgcatggaagaatggttttgaacgacaagatgtctccggcgtcaatttctttgagatgcatacaatacgaactgccaccaataacttcagtccttcaaataaacttggtcaaggtggatttggtccagtttacaagggaaagttggtagatgggaaggaaataggtgttaaacgccttgctagtagttccggtcagggtacagaggagttcatgaatgaaataacactgatctcaaaattgcaacacagaaacttggttcggcttttgggatactgcatcgacggagaagagaagctactgatctatgagttcatggtgaacaaaagtctcgatatttttatatttgatccatgtctaaagtttgagcttgattggccaaagagatttaatatcattcaaggtattgcgcgtggacttctctatctccaccgtgactcacgcctcagagttattcaccgagacctgaaggtcagcaatattcttctggacgacagaatgaatccaaaaatatcagattttggattggctcggatgttccagggaacccaatatcaagacaacactcgcagagttgtaggaactctaggatatatgtctcccgagtatgcatgggcaggattgttctctgagaagtctgatatctatagcttcggagttctgatgttagaaatcatcagcggaaagagaatttcacggttcatctatggtgatgaaagcaaaggccttcttgcttatacatgggattcttggtgcgagaccgggggatcaaatctacttgacagagatcttactgatacatgccaagcattcgaagttgcgagatgtgttcagattggtctgctctgtgttcaacacgaagccgtcgacagacccaacactcttcaagtactatccatgcttacttcagcaacagatcttccagtaccaaaacagccaatatttgcagtgcatactctaaacgatatgccaatgttacaggctaattctcaagatttcttatctgtaaatgagatgacagaatctatgatccaaggaaggtaa